SEQUENCE LISTING (6 sequences)

SEQ ID NO: 1 - DNA, 42 bp, Artificial Sequence
  5-HT7R-NheI forward primer
cgacgtgctagcgccaccatgtacccatacgatgttccagat

SEQ ID NO: 2 - DNA, 41 bp, Artificial Sequence
  5-HT7R-EcoRI reverse primer
ctgagcgaattcgtgatgaatcatgacctttttttctacag

SEQ ID NO: 3 - protein, 19 aa, artificial sequence
  C-Tail
GMHEALKLAERPERPEFVL

SEQ ID NO: 4 - protein, 19 aa, artificial sequence
  MutT1
GMHEALKLAERAERPEFVL

SEQ ID NO: 5 - protein, 19 aa, artificial sequence
  MutT2
GMHEALKLAERAERAEFVL

SEQ ID NO: 6 - protein, 15 aa, artificial sequence
  MutT3
GMHEALKLAEREFVL

(19) United States
(12) Patent Application Publication
(10) Pub. No.: US 2024/0199555 A1
(43) Pub. Date: Jun. 20, 2024

(54) APPLICATIONS OF BIASED LIGANDS OF THE SEROTONIN 5-HT7 RECEPTOR FOR THE TREATMENT OF PAIN, MULTIPLE SCLEROSIS AND THE CONTROL OF THERMOREGULATION

(71) Applicants: Centre National de la Recherche Scientifique, PARIS (FR); Université d'Orléans, ORLEANS (FR)

(72) Inventors: Séverine MORISSET-LOPEZ, ST PRYVE ST MESMIN (FR); Franck SUZENET, SAINT JEAN LE BLANC (FR); Gérald GUILLAUMET, SAINT JEAN LE BLANC (FR); Emmanuel DEAU, ROSCOFF (FR); Elodie ROBIN, LIGNIERES (FR); Chayma EL KHAMLICHI, BOIGNY SUR BIONNE (FR); Flora REVERCHON-ASSADI, FAY AUX LOGES (FR); Nadège HERVOUET-COSTE, JOUY LE POTIER (FR); Fahima MADOURI, SAINT-JEAN-DE-BRAY (FR); Marie-Aude HIEBEL, OLIVET (FR); Julie LE-BESCONT, SAINT GREGOIRE (FR)

(21) Appl. No.: 18/551,361

(22) PCT Filed: Mar. 18, 2022
(86) PCT No.: PCT/EP2022/057227

§ 371 Date: Sep. 19, 2023

(30) Foreign Application Priority Data
Mar. 19, 2021 (EP) 21315045.1

(51) Int. Cl. C07D 235/26; A61K 31/496; A61P 29/00; C07D 233/36; C07D 401/12; C07D 403/12; C07D 487/10

(52) U.S. Cl. CPC C07D 235/26; A61K 31/496; A61P 29/00; C07D 233/36; C07D 401/12; C07D 403/12; C07D 487/10

(57) ABSTRACT

The present invention relates to a compound having the following formula (1)

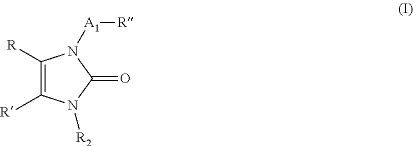

wherein:
   R and R′ are, independently from each other, H or (C₁-C₆)alkyl groups, or form together with the carbon atoms carrying them a (C₆-C₁₀)aryl group;
   R₂ is selected from the group consisting of: H, (C₁-C₆)alkyl group, halo(C₁-C₆)alkyl group, aryl, and heteroaryl;
   A₁ is a linker;
   R″ is either a group (A-1) or a group (A-2)
for use in the treatment of a brain disorder involving modified 5-HT7R-mediated signaling, especially for use in the treatment of pain or inflammation or in the treatment of multiple sclerosis, or for use to induce hypothermia.

### CROSS-REFERENCE TO RELATED APPLICATIONS

The present application is a filing under 35 U.S.C. 371 as the National Stage of International Application No. PCT/EP2022/057227, filed Mar. 18, 2022, entitled “APPLICATIONS OF BIASED LIGANDS OF THE SEROTONIN 5-HT7 RECEPTOR FOR THE TREATMENT OF PAIN, MULTIPLE SCLEROSIS AND THE CONTROL OF THERMOREGULATION,” which claims priority to European Application No. 21315045.1 filed with the Intellectual Property Office of Europe on Mar. 19, 2021, both of which are incorporated herein by reference in their entirety for all purposes.

### INCORPORATION BY REFERENCE OF MATERIAL IN ASCII TEXT FILE

This application incorporates by reference the Sequence Listing contained in the following ASCII text file being submitted concurrently herewith:

File name: 4692-12800 BNT241755USPC Sequence Listing; created on Sep. 15, 2023; and having a file size of 1.75 KB.

The information in the Sequence Listing is incorporated herein in its entirety for all purposes.

The present invention concerns biased ligands of the serotonin 5-HT₇ receptor for their use in the treatment of pain or multiple sclerosis, or to induce hypothermia.

Among 14 serotonin receptor subtypes, 5-HT₇ receptors (5-HT₇R) belong to the GPCR family or so called seven transmembrane-spanning receptor. 5-HT₇R couples to the heterotrimeric G protein Gs, which in turn activates different adenylate cyclase isoforms and increases cAMP production in several recombinant systems as well as in native systems. Elevated levels of cAMP induce the activation of cAMP-dependent protein kinase (PKA), which in turn has cell type-specific effects on MAPK cascade. It was shown that stimulation of 5-HT₇R by agonists induces ERK1/2 activation in both transfected HEK-293 cells and in native systems. 5-HT₇R are expressed in the peripheral and central nervous system with highest densities in thalamus, hypothalamus, cerebral cortex, amygdala and striatal complex (Kobe, F., Guseva, D., Jensen, T. P., Wirth, A., Renner, U., Hess, D., Muller, M., Medrihan, L., Zhang, W., Zhang, M., Braun, K., Westerholz, S., Herzog, A., Radyushkin, K., EI-Kordi, A., Ehrenreich, H., Richter, D. W., Rusakov, D. A., and Ponimaskin, E. (2012) 5-HT7R/G12 signaling regulates neuronal morphology and function in an age-dependent manner. J Neurosci 32, 2915-2930). Numerous data have established 5-HT₇R implication in the control of circadian rhythms and thermoregulation, learning and memory as well as in CNS disorders such as depression, Alzheimer's disease and schizophrenia. To date, 5-HT₇R ligands have been classified according to their activity on Gs protein coupling, the two main classes being agonists (5-CT, AS-19, E55888, 80HDPAT and LP-211) and antagonists (SB269970, DR4004 and EGIS (compound 9e′ from J. Med. Chem. 2008, 51, 2522) and JNJ18038683). The use of these ligands provided a better understanding of the role of the receptor in both health and diseases. In particular, numerous studies have investigated their therapeutic potential in the treatment of pain.

The identification of 5-HT₇R biased ligand may help in better understanding the relationship between therapeutic effects and molecular mode of action of these ligands.

In contrast to standard agonists and antagonists which activate or inactivate the entirety of a receptor's signaling network, biased ligands are capable of stabilizing subsets of receptor conformations, hence eliciting selective modulation within the network. The concept of functional selectivity of a ligand has recently emerged as an interesting property in drug discovery. Increasing preclinical data highlight the value of using such ligands, which exhibit a unique spectrum of pharmacological responses, for instance by specifically targeting G protein- or β-arrestin-dependent signaling. Biased ligands by selectively modulating a subset of receptor functions may optimize therapeutic action and generate less pronounced side effects than compounds globally affecting receptor activity (Wisler, J. W., Rockman, H. A., and Lefkowitz, R. J. (2018) Biased G Protein-Coupled Receptor Signaling: Changing the Paradigm of Drug Discovery. Circulation 137, 2315-2317). Although binding of β-arrestins to the GPCR has been primarily involved in the termination of G protein signaling by inducing desensitization and internalization of the receptor, in the last two decades, numerous studies indicated that β-arrestins can be intimately involved in additional signaling events through dependent or independent G protein coupling (Gurevich, V. V., and Gurevich, E. V. (2020) Biased GPCR signaling: Possible mechanisms and inherent limitations. Pharmacol Ther 211, 107540). It is now appreciated that β-arrestins can initiate their own signalling, such as transactivation of EGFR, induction of ERK1/2 pathway or activation of CaM-KII which can produce specific cellular responses. Several β-arrestin-biased ligands have been identified and showed therapeutic interest (Whalen, E. J., Rajagopal, S., and Lefkowitz, R. J. (2011) Therapeutic potential of beta-arrestin- and G protein-biased agonists. Trends Mol Med 17, 126-139). There is thus to date a need for β-arrestin biased 5-HT₇R ligands that may be used for example for the treatment of pain.

The aim of the present invention is to provide compounds being β-arrestin biased 5-HT₇R ligands.

Another aim of the present invention is to provide β-arrestin biased 5-HT₇R ligands useful for inducing hypothermia or for the treatment of a brain disorder involving modified 5-HT7R-mediated signaling.

Therefore, the present invention relates to a compound having the following formula (I)

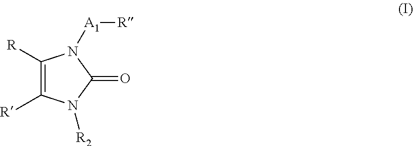

wherein:
   R and R′ are, independently from each other, H or (C₁-C₆)alkyl groups, or form together with the carbon atoms carrying them a (C₆-C₁₀)aryl group;
said aryl group being optionally substituted with one or several substituents, said substituents being in particular selected from the group consisting of:
   halogen;
   (C₁-C₆)alkyl;
   OH;
   (C₁-C₆)alkoxy;
   —NR_{d}Rₑ, R_{d} and Rₑ, independently from each other, being H or a (C₁-C₆)alkyl group;
   aryl;
   heteroaryl;
   halo(C₁-C₆)alkyl group, such as CF₃; —C(═O)—NR_{f}R_{g}, R_{f} and R_{g}, independently from each other, being H or a (C₁-C₆)alkyl group; and
   —C(═O)—Rₕ, Rₕ being a (C₁-C₆)alkyl group;
   R₂ is selected from the group consisting of:
      H;
      (C₁-C₆)alkyl group;
      halo(C₁-C₆)alkyl group;
      aryl; and
   heteroaryl;
   A₁ is a linker having the following formula (II):

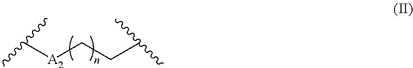

wherein:
   n is an integer varying from 1 to 7; and
   A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
   R″ is:
      either a group having the following formula (A-1):

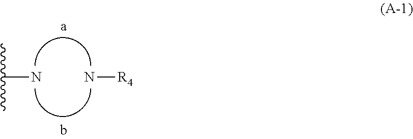

wherein:
   the bonds “a” and “b” form a 4- to 10-membered saturated heterocycloalkyl group with the nitrogen atoms carrying them, said heterocycloalkyl group being optionally substituted for example with at least one substituent selected from (C₁-C₆)alkyl groups, and being selected from the monocyclic groups, bicyclic groups, fused bicycles and spiro-type rings; and
   R₄ is selected from the optionally substituted (C₆-C₁₀)aryl and heteroaryl groups;
   either a group having the following formula (A-2):

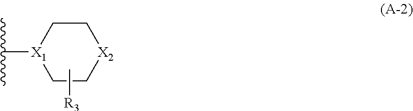

wherein:
   X₁ is —N— or —CH—;
   X₂ is selected from the group consisting of:
      a group —X₁—R₄, X₁ being as defined above and R₄ being selected from the optionally substituted (C₆-C₁₀)aryl and heteroaryl groups; and
      a group —CH—CO—Ar, Ar having the below formula (III):

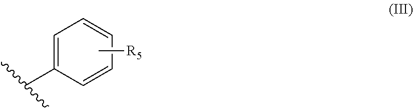

R₅ being selected from the group consisting of:
   H;
   halogen;
   (C₁-C₆)alkyl;
   halo(C₁-C₆)alkyl;
   hetero(C₁-C₆)alkyl;
   OH;
   (C₁-C₆)alkoxy;
   halo(C₁-C₆)alkoxy;
   CN;
   —C(═O)—Rᵢ, Rᵢ being a (C₁-C₆)alkyl group;
   —SO₂—NRⱼRₖ, Rⱼ and Rₖ, independently from each other, being H or a (C₁-C₆)alkyl group;
   —NR_{b}R_{c}, R_{b} and R_{c}, independently from each other, being H or a (C₁-C₆)alkyl group; and
   optionally substituted (C₆-C₁₀)aryl and heteroaryl, said aryl or heteroaryl being possible fused with the phenyl ring carrying them; and
   R₃ is selected from the group consisting of:
      H;
      (C₁-C₆)alkyl group; and
      hetero(C₁-C₆)alkyl group;
or its pharmaceutically acceptable salts, racemates, diastereomers or enantiomers,
for use in the treatment of a brain disorder involving modified 5-HT7R-mediated signaling or for use to induce hypothermia.

Within the present invention, the term “brain disorders involving a modified 5-HT7R-mediated signaling” refers to a modification of 5-HT7R expression and/or 5-HT7R signaling pathways mediated by G proteins activation and/or by alternative mechanisms where β-arrestins are involved.

Within the present invention, the term “β-arrestins biased ligands” refers to molecules acting as antagonist on cAMP pathway (block Gs signaling) and as agonist on ERK pathway through the recruitment of β-arrestins and by activation of Src kinase.

In particular, the brain disorder according to the invention is the pain or the multiple sclerosis.

According to an embodiment, the compound of formula (I) above are used for the treatment of pain or inflammation or in the treatment of multiple sclerosis, or for use to induce hypothermia.

The present invention also relates to compounds of formula (I) as such, as well as to medicaments or pharmaceutical compositions comprising said compounds, or to the compounds of formula (I) for use as a drug.

The following definitions are set forth to illustrate and define the meaning and scope of the various terms used to describe the invention herein.

The expression “Cₜ-C_{z}” means a carbon-based chain which can have from t to z carbon atoms, for example C₁-C₃ means a carbon-based chain which can have from 1 to 3 carbon atoms.

The term “alkyl group” means: a linear or branched, saturated, hydrocarbon-based aliphatic group comprising, unless otherwise mentioned, from 1 to 12 carbon atoms. By way of examples, mention may be made of methyl, ethyl, n-propyl, isopropyl, butyl, isobutyl, tert-butyl or pentyl groups.

The term “aryl group” means: a cyclic aromatic group comprising between 6 and 10 carbon atoms. By way of examples of aryl groups, mention may be made of phenyl or naphthyl groups.

The term “heteroaryl group” means: a 5- to 10-membered aromatic monocyclic or bicyclic group containing from 1 to 4 heteroatoms selected from O, S or N. By way of examples, mention may be made of imidazolyl, thiazolyl, oxazolyl, furanyl, thiophenyl, pyrazolyl, oxadiazolyl, tetrazolyl, pyridinyl, pyrazinyl, pyrimidinyl, pyridazinyl, indolyl, benzofuranyl, benzothiophenyl, benzoxazolyl, benzimidazolyl, indazolyl, benzothiazolyl, isobenzothiazolyl, benzotriazolyl, quinolinyl, isoquinolinyl, and triazinyl groups.

By way of a heteroaryl comprising 5 to 6 atoms, including 1 to 4 nitrogen atoms, mention may in particular be made of the following representative groups: pyrrolyl, pyrazolyl, 1,2,3-triazolyl, 1,2,4-triazolyl, tetrazolyl and 1,2,3-triazinyl.

Mention may also be made, by way of heteroaryl, of thiophenyl, oxazolyl, furazanyl, 1,2,4-thiadiazolyl, naphthyridinyl, quinoxalinyl, phthalazinyl, imidazo[1,2-a]pyridine, imidazo[2,1-b]thiazolyl, cinnolinyl, benzofurazanyl, azaindolyl, benzimidazolyl, benzothiophenyl, thienopyridyl, thienopyrimidinyl, pyrrolopyridyl, imidazopyridyl, benzoazaindole, 1,2,4-triazinyl, indolizinyl, isoxazolyl, isoquinolinyl, isothiazolyl, purinyl, quinazolinyl, quinolinyl, isoquinolyl, 1,3,4-thiadiazolyl, thiazolyl, isothiazolyl, carbazolyl, and also the corresponding groups resulting from their fusion or from fusion with the phenyl nucleus.

The term “heterocycloalkyl group” means: a 4- to 10-membered, saturated or partially unsaturated, monocyclic or bicyclic group comprising from one to three heteroatoms selected from O, S or N; the heterocycloalkyl group may be attached to the rest of the molecule via a carbon atom or via a heteroatom; the term bicyclic heterocycloalkyl includes fused bicycles and spiro-type rings.

By way of saturated heterocycloalkyl comprising from 5 to 6 atoms, mention may be made of oxetanyl, tetrahydrofuranyl, dioxolanyl, pyrrolidinyl, azepinyl, oxazepinyl, pyrazolidinyl, imidazolidinyl, tetrahydrothiophenyl, dithiolanyl, thiazolidinyl, tetrahydropyranyl, tetrahydropyridinyl, dioxanyl, morpholinyl, piperidinyl, piperazinyl, tetrahydrothiopyranyl, dithianyl, thiomorpholinyl or isoxazolidinyl.

Among the heterocycloalkyls, mention may also be made, by way of examples, of bicyclic groups such as (8aR)-hexahydropyrrolo[1,2-a]pyrazin-2(1H)-yl, octahydroindozilinyl, diazepanyl, dihydroimidazopyrazinyl and diazabicycloheptanyl groups, or else diazaspiro rings such as 1,7-diazaspiro[4.4]non-7-yl or 1-ethyl-1,7-diazaspiro[4.4]non-7-yl.

When the heterocycloalkyl is substituted, the substitution(s) may be on one (or more) carbon atom(s) and/or on the heteroatom(s). When the heterocycloalkyl comprises several substituents, they may be borne by one and the same atom or different atoms.

The term “cycloalkyl group” means: a cyclic carbon-based group comprising, unless otherwise mentioned, from 3 to 6 carbon atoms. By way of examples, mention may be made of cyclopropyl, cyclobutyl, cyclopentyl, cyclohexyl, etc. groups.

When an alkyl radical is substituted with an aryl group, the term “arylalkyl” or “aralkyl” radical is used. The “arylalkyl” or “aralkyl” radicals are aryl-alkyl-radicals, the aryl and alkyl groups being as defined above. Among the arylalkyl radicals, mention may in particular be made of the benzyl or phenethyl radicals.

The term “halogen” means: a fluorine, a chlorine, a bromine or an iodine.

The term “alkoxy group” means: an —O-alkyl radical where the alkyl group is as previously defined. By way of examples, mention may be made of —O—(C₁-C₄)alkyl groups, and in particular the —O-methyl group, the —O-ethyl group as —O—C₃alkyl group, the —O-propyl group, the —O-isopropyl group, and as —O—C₄alkyl group, the —O-butyl, —O-isobutyl or —O-tert-butyl group.

The above mentioned “alkyl”, “cycloalkyl”, “aryl”, “heteroaryl” and “heterocycloalkyl” radicals can be substituted with one or more substituents. Among these substituents, mention may be made of the following groups: amino, hydroxyl, thiol, oxo, halogen, alkyl, alkoxy, alkylthio, alkylamino, aryloxy, arylalkoxy, cyano, trifluoromethyl, carboxy or carboxyalkyl.

The term “alkylthio” means: an —S-alkyl group, the alkyl group being as defined above.

The term “alkylamino” means: an —NH-alkyl group, the alkyl group being as defined above.

The term “aryloxy” means: an —O-aryl group, the aryl group being as defined above.

The term “arylalkoxy” means: an aryl-alkoxy-group, the aryl and alkoxy groups being as defined above.

The term “carboxyalkyl” means: an HOOC-alkyl-group, the alkyl group being as defined above.

As examples of carboxyalkyl groups, mention may in particular be made of carboxymethyl or carboxyethyl.

The term “haloalkyl group” means: an alkyl group as defined above, in which one or more of the hydrogen atoms is (are) replaced with a halogen atom. By way of example, mention may be made of fluoroalkyls, in particular CF₃ or CHF₂.

The term “haloalkoxy group” means: an —O-haloalkyl group, the haloalkyl group being as defined above. By way of example, mention may be made of fluoroalkyls, in particular OCF₃ or OCHF₂.

The term “heteroalkyl group” means: an alkyl group as defined above, in which one or more of the carbon atoms is (are) replaced with a heteroatom, such as O or N.

The term “carboxyl” means: a COOH group.

The term “oxo” means: “═O”.

In some embodiments of the invention, the compounds of the invention can contain one or more asymmetric centers and thus occur as racemates and racemic mixtures, single enantiomers, individual diastereoisomeric mixtures. All such isomeric forms of these compounds are included in the present invention, unless expressly provided otherwise.

In some embodiments, the compounds of the invention can contain one or more double bonds and thus occur as individual or mixtures of Z and/or E isomers. All such isomeric forms of these compounds are included in the present invention, unless expressly provided otherwise.

In the embodiments where the compounds of the invention can contain multiple tautomeric forms, the present invention also includes all tautomeric forms of said compounds unless expressly provided otherwise.

According to an embodiment in formula (I) as defined above, R and R′ are H.

According to an embodiment in formula (I) as defined above, R and R′ form together with the carbon atoms carrying them a (C₆-C₁₀)aryl group, in particular a fused phenyl group, said phenyl group being optionally substituted with one or several substituents as defined above.

According to an embodiment, in formula (I), A₁ is a linear or branched alkylene bond comprising from 2 to 10 carbon atoms.

According to an embodiment, in formula (I), A₁ is an alkylene bond of formula —(CH₂)ₙ—, n being as defined above. Preferably, n is an integer varying from 2 to 7.

According to an embodiment, in formula (I), A₁ is a linker of formula (II) as defined above wherein A₂ is a C₂ divalent radical, possibly substituted with at least one substituent as defined above in formula (II).

According to an embodiment, in formula (I), A₁ is a linker of formula (II) as defined above wherein A₂ is a C₂ divalent radical, wherein possibly at least one carbon atom of A₂ is replaced with —O—.

According to an embodiment, in formula (I), A₁ is a linker of formula (II) as defined above wherein A₂ is a C₂ divalent radical, possibly substituted with at least one (C₁-C₆)alkyl.

According to an embodiment, in formula (I), R″ is a group having the following (A-1) as defined above. In such embodiment, R″ is a 4- to 10-membered saturated heterocycloalkyl group including at least two nitrogen atoms, said heterocycloalkyl group being selected from the monocyclic groups, bicyclic groups, fused bicycles and spiro-type rings, said heterocycloalkyl group being linked to a R₄ group as defined above.

As examples of monocyclic groups, bicyclic groups, fused bicycles and spiro-type rings for R″, the followings may be mentioned:

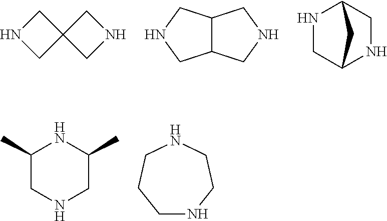

According to an embodiment, in formula (I) as defined above, R″ is a group having the formula (A-1), wherein R₄ is selected from the (C₆-C₁₀)aryl and heteroaryl groups, optionally substituted with one or several substituents selected from the group consisting of: H, (C₁-C₆)alkyl, —OH, (C₁-C₆)alkoxy, halogen, thio(C₁-C₆)alkyl, halo(C₁-C₆)alkyl, halo(C₁-C₆)alkoxy, and —NR_{b}R_{c}, R_{b} and R_{c}, independently from each other, being H or a (C₁-C₆)alkyl group.

According to an embodiment, in formula (I) as defined above, R″ is a group having the formula (A-1), wherein R₄ is a (C₆-C₁₀)aryl group, optionally substituted with one or several substituents selected from the group consisting of: H, (C₁-C₆)alkyl, —OH, (C₁-C₆)alkoxy, halogen, thio(C₁-C₆)alkyl, halo(C₁-C₆)alkyl, halo(C₁-C₆)alkoxy, and —NR_{b}R_{c}, R_{b} and R_{c}, independently from each other, being H or a (C₁-C₆)alkyl group. According to an embodiment, in formula (I) as defined above, R″ is a group having the formula (A-1), wherein R₄ is a (C₆-C₁₀)aryl group, substituted with one or several substituents, for example one or two substituents, said substituents being selected from the group consisting of: (C₁-C₆)alkyl, —OH, halogen, and halo(C₁-C₆)alkyl.

The present invention also relates to a compound having the following formula (I′):

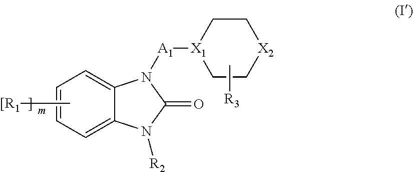

wherein:
   m is an integer comprised from 1 to 4;
   each R₁, identical or different, is selected from the group consisting of:
      H;
      halogen;
      (C₁-C₆)alkyl;
      OH;
      (C₁-C₆)alkoxy;
      —NR_{d}Rₑ, R_{d} and Rₑ, independently from each other, being H or a (C₁-C₆)alkyl group;
      aryl;
      heteroaryl;
      halo(C₁-C₆)alkyl group such as CF₃;
      —C(═O)—NR_{f}R_{g}, R_{f} and R_{g}, independently from each other, being H or a (C₁-C₆)alkyl group; and
      —C(═O)—Rₕ, Rₕ being a (C₁-C₆)alkyl group;
   R₂ is selected from the group consisting of:
      H;
      (C₁-C₆)alkyl group;
      halo(C₁-C₆)alkyl group;
      aryl; and
      heteroaryl;
   A₁ is a linker having the following formula (II):

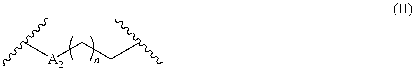

wherein:
   n is an integer varying from 1 to 7; and
   A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ is replaced with a heteroatom such as —O—, —S— or —NRₐ—, Rₐ being H or a (C₁-C₆)alkyl group;
   X₁ is —N— or —CH—;
   X₂ is selected from the group consisting of:
      a group —X₁—R₄, X₁ being as defined above and R₄ being selected from the optionally substituted (C₆-C₁₀)aryl and heteroaryl groups; and
      a group —CH—CO—Ar, Ar having the below formula (III):

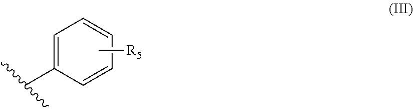

R₅ being selected from the group consisting of:
   H;
   halogen;
   (C₁-C₆)alkyl;
   halo(C₁-C₆)alkyl;
   hetero(C₁-C₆)alkyl;
   OH;
   (C₁-C₆)alkoxy;
   halo(C₁-C₆)alkoxy;
   CN;
   —C(═O)—Rᵢ, Rᵢ being a (C₁-C₆)alkyl group;
   —SO₂—NRⱼRₖ, Rⱼ and Rₖ, independently from each other, being H or a (C₁-C₆)alkyl group;
   —NR_{b}R_{c}, R_{b} and R_{c}, independently from each other, being H or a (C₁-C₆)alkyl group; and
   optionally substituted (C₆-C₁₀)aryl and heteroaryl, said aryl or heteroaryl being possible fused with the phenyl ring carrying them; and
   R₃ is selected from the group consisting of:
      H;
      (C₁-C₆)alkyl group; and
      hetero(C₁-C₆)alkyl group;
for use in the treatment of pain or in the treatment of multiple sclerosis, or for use to induce hypothermia.

The present invention also relates to a compound having the following formula (I′):

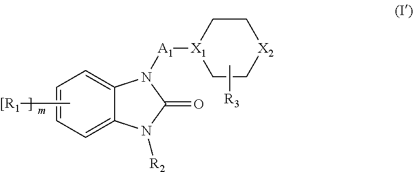

wherein:
   m is an integer comprised from 1 to 4;
   each R₁, identical or different, is selected from the group consisting of:
      H;
      halogen;
      (C₁-C₆)alkyl; OH;
      (C₁-C₆)alkoxy;
      —NR_{d}Rₑ, R_{d} and Rₑ, independently from each other, being H or a (C₁-C₆)alkyl group;
      aryl;
      heteroaryl;
      halo(C₁-C₆)alkyl group such as CF₃;
      —C(═O)—NR_{f}R_{g}, R_{f} and R_{g}, independently from each other, being H or a (C₁-C₆)alkyl group; and
      —C(═O)—Rₕ, Rₕ being a (C₁-C₆)alkyl group;
   R₂ is selected from the group consisting of:
      H;
      (C₁-C₆)alkyl group;
      halo(C₁-C₆)alkyl group;
      aryl; and
      heteroaryl;
   A₁, X₁, X₂, and R₃ are as defined above in formula (I),
for use in the treatment of pain or in the treatment of multiple sclerosis, or for use to induce hypothermia.

According to an embodiment, in formula (I′), m=1.

According to an embodiment, in formula (I′), m=1 and R₁ is H.

According to an embodiment, in formula (I′), m=1 and R₁ is halogen.

According to an embodiment, a family of compounds for the use according to the present invention consists of compounds having the following formula (IV):

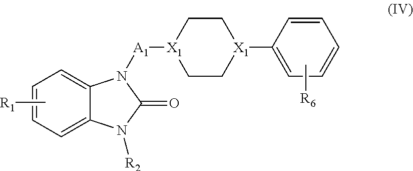

wherein:
   R₁, R₂, A₁, and X₁ are as defined above; and
   R₆ is selected from the group consisting of: H, (C₁-C₆)alkyl, —OH, (C₁-C₆)alkoxy, halogen, thio(C₁-C₆)alkyl, halo(C₁-C₆)alkyl, halo(C₁-C₆)alkoxy, and —NR_{b}R_{c}, R_{b} and R_{c}, independently from each other, being H or a (C₁-C₆)alkyl group;

Preferably, in formula (IV), X₁ is —N—.

According to an embodiment, a family of compounds for the use according to the present invention consists of compounds having the following formula (IV-1):

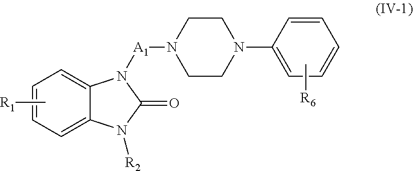

R₁, R₂, A₁, and R₆ being as defined above in formula (IV).

Preferably, in formula (IV) or in formula (IV-1), R₆ is H, OH, halogen, thio(C₁-C₆)alkyl or (C₁-C₆)alkoxy.

According to an embodiment, a family of compounds for the use according to the present invention consists of compounds having the following formula (V):

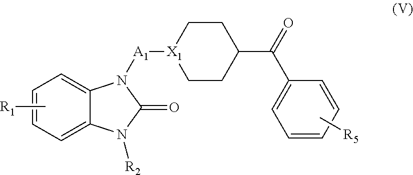

wherein R₁, R₂, A₁, X₁, and R₆ are as defined above.

Preferably, in formula (V), R₆ is H or halogen.

A sub-family of compounds for the use according to the present invention consists of compounds having the above formula (V), wherein X₁ is —N—.

According to an embodiment, a family of compounds for the use according to the present invention consists of compounds having the following formula (V-1):

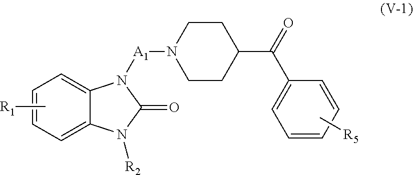

wherein R₁, R₂, A₁, and R₅ are as defined above.

Preferably, in this subfamily of compounds, R₅ is H or halogen.

According to an embodiment, in formula (I), R₁ is H or a halogen atom.

According to an embodiment, in formula (I′), (IV), (IV-1), (V) or (V-1), R₁ is H or a halogen atom.

According to an embodiment, in formula (I), R₂ is H or a (C₁-C₆)alkyl group.

According to an embodiment, in formula (I′), (IV), (IV-1), (V) or (V-1), R₂ is H or a (C₁-C₆)alkyl group.

According to an embodiment, in formula (I), A₁ is a (C₂-C₇)alkylene radical.

According to an embodiment, in formula (I′), (IV), (IV-1), (V) or (V-1), A₁ is a (C₂-C₇)alkylene radical.

As compounds for the use according to the present invention, one may cite the compounds disclosed in the article of Deau et al. “Rational Design, Pharmacomodulation, and Synthesis of Dual 5-Hydroxytryptamine 7 (5-HT₇)/5-Hydroxytryptamine 2A (5-HT_{2A}) Receptor Antagonists and Evaluation by [¹⁸F]-PET Imaging in a Primate Brain”, Journal of Medicinal Chemistry, Vol. 58, pp 8066-8096.

According to an embodiment, a family of compounds for the use according to the present invention consists of compounds having the following formula (VI):

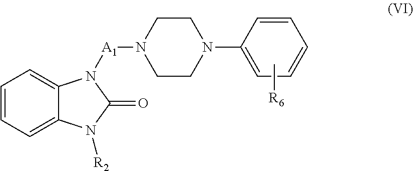

wherein:
   R₂ and A₁ are as defined above; and
   R₆ is selected from the group consisting of: H, —OH, (C₁-C₆)alkoxy, halogen, and thio(C₁-C₆)alkyl.

Preferably, in formula (VI), R₂ is H or a (C₁-C₆)alkyl group, such as a n-butyl group.

Preferably, in formula (VI), A₁ is a C₄ or C₅ alkylene radical.

Preferably, in formula (VI), R₂ is H or a (C₁-C₆)alkyl group, such as a n-butyl group, and A₁ is a C₄ or C₅ alkylene radical.

Preferably, in formula (VI), R₆ is H, 4-Cl, 4-OMe, 2-SMe, 4-Br, 4-I, 4-F or 4-Cl.

According to an embodiment, a family of compounds for the use according to the present invention consists of compounds having the following formula (VII):

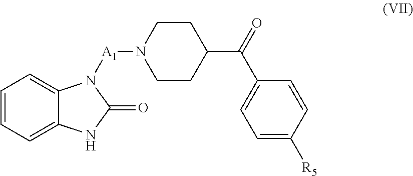

wherein A₁ and R₆ are as defined above.

Preferably, in formula (VII), R₆ is halogen, and preferably F.

Preferably, in formula (VII), A₁ is a (C₂-C₇)alkylene radical.

Preferably, in formula (VII), R₅ is halogen and A₁ is a (C₂-C₇)alkylene radical.

Preferably, in formula (VI), R₅ is F.

According to a preferred embodiment, the compounds for the use according to the invention are selected from the following compounds:

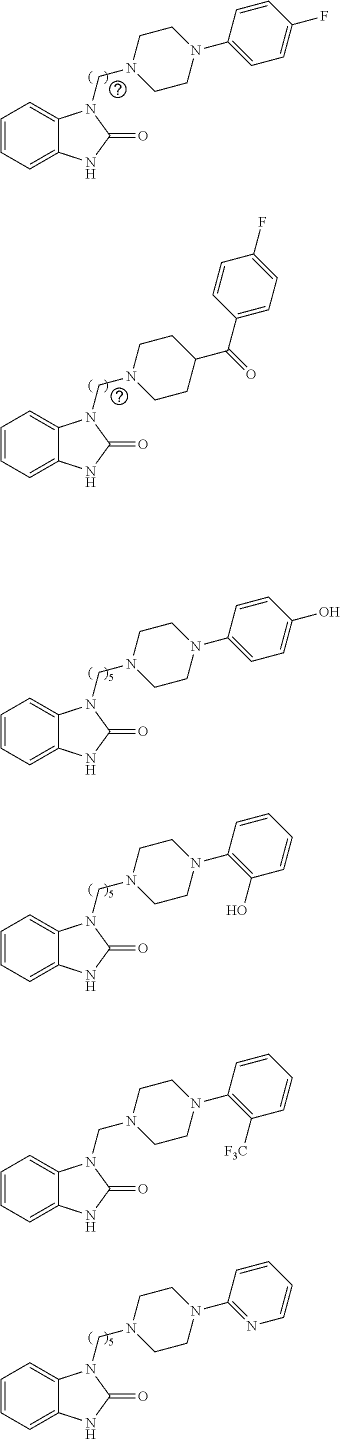

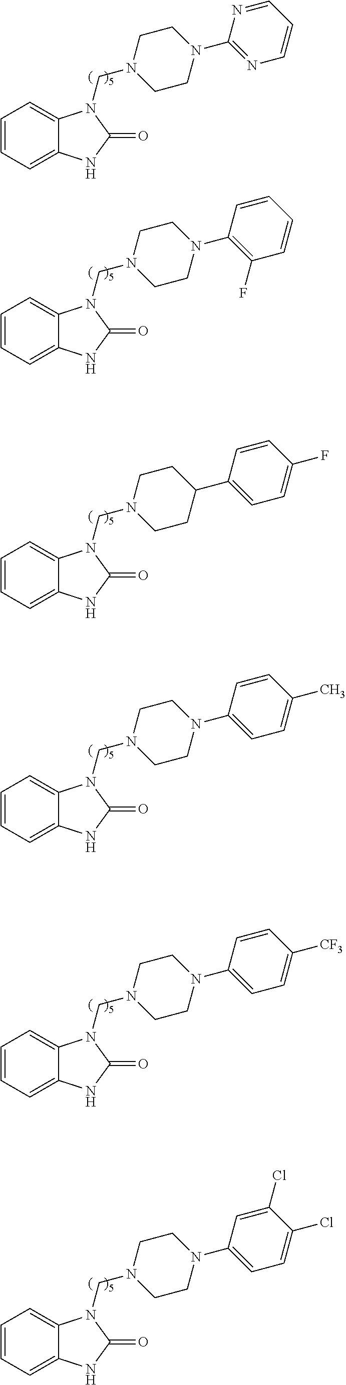

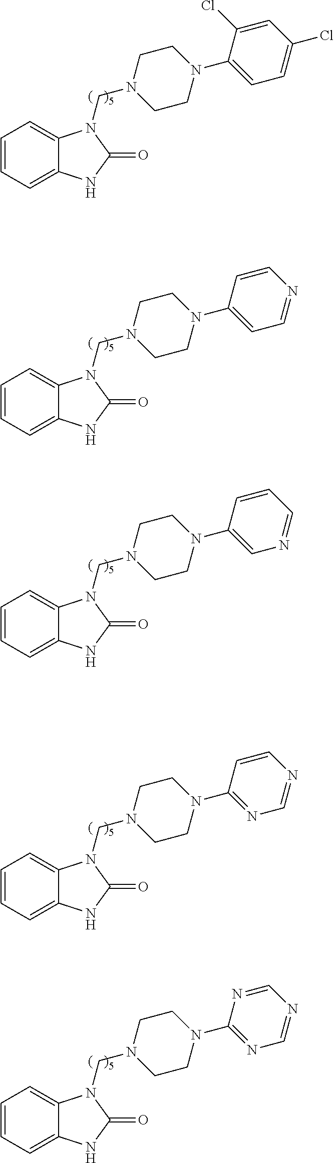

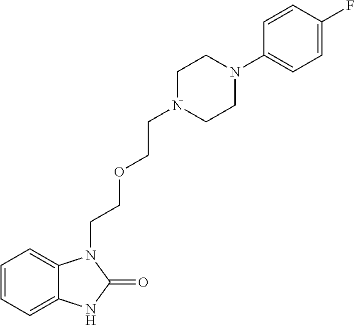 [SMILES: O=c1[nH]c2ccccc2n1CCOCCN1CCN(c2ccc(F)cc2)CC1]

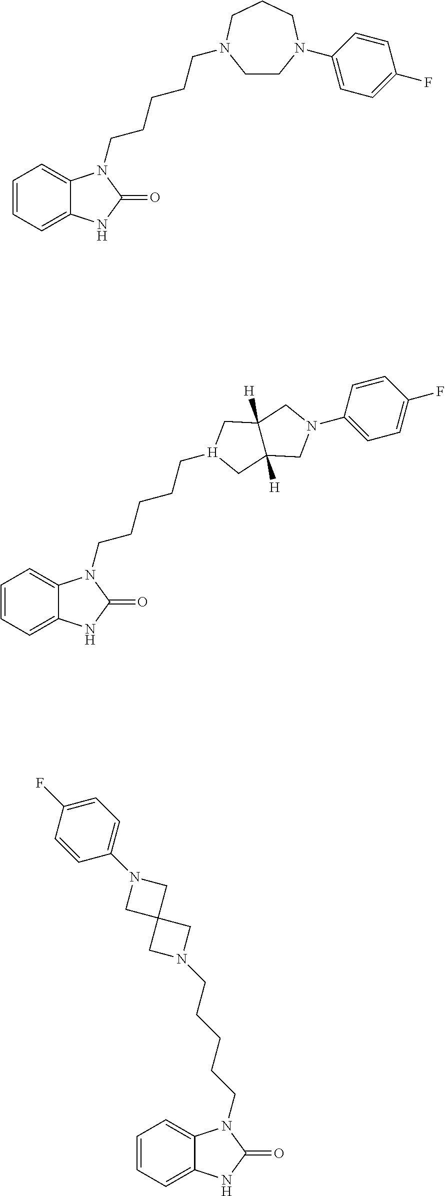

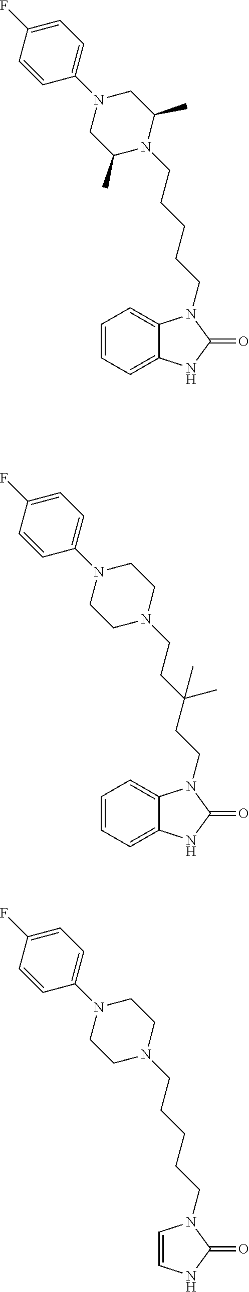 [SMILES: C[C@@H]1CN(c2ccc(F)cc2)C[C@H](C)N1CCCCCn1c(=O)[nH]c2ccccc21 ; CC(C)(CCN1CCN(c2ccc(F)cc2)CC1)CCn1c(=O)[nH]c2ccccc21 ; O=c1[nH]ccn1CCCCCN1CCN(c2ccc(F)cc2)CC1]

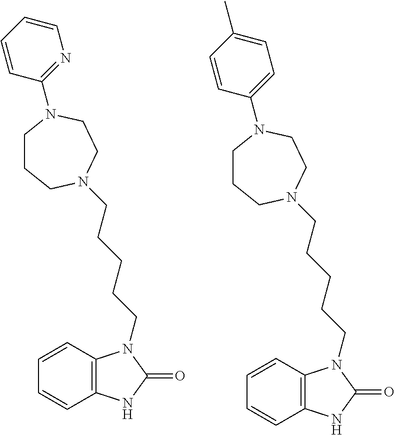 [SMILES: O=c1[nH]c2ccccc2n1CCCCCN1CCCN(c2ccccn2)CC1 ; Cc1ccc(N2CCCN(CCCCCn3c(=O)[nH]c4ccccc43)CC2)cc1]

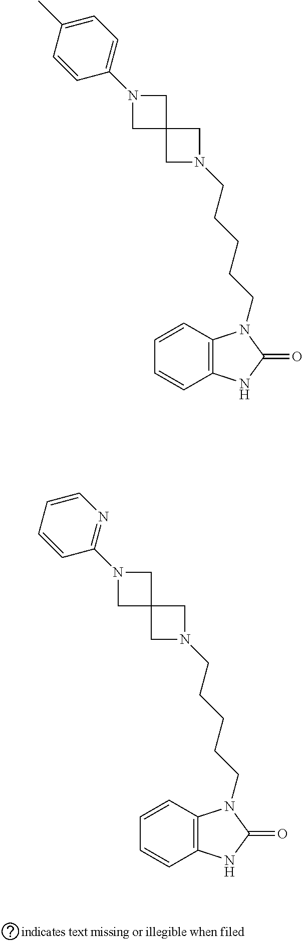 [SMILES: Cc1ccc(N2CC3(CN(CCCCCn4c(=O)[nH]c5ccccc54)C3)C2)cc1 ; O=c1[nH]c2ccccc2n1CCCCCN1CC2(C1)CN(c1ccccn1)C2]

According to an embodiment, the present invention relates to a compound as defined above, for use to reduce pain, for use for treating inflammation or for use for treating multiple sclerosis or to reduce the body temperature in a mammalian subject.

Preferably, the pain is selected from the group consisting of: pain from thermic, mechanic, or inflammatory stimulus, acute and tonic pain, inflammatory pain, visceral pain, neuropathic pain, and post-operative pain.

According to the invention, the compounds may be used in pharmaceutical compositions for oral, sublingual, subcutaneous, intramuscular, intravenous, topical, local, intratracheal, intranasal, transdermal or rectal administration, the active ingredient of formula (I), above, or the salt thereof, can be administered in unit administration form, as a mixture with conventional pharmaceutical excipients, to animals and to human beings for the treatment of the disorders and diseases as mentioned above.

The suitable unit administration forms include oral forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms, and implants. For topical application, the compounds according to the invention can be used in creams, gels, ointments or lotions.

According to the usual practice, the dosage suitable for each patient is determined by the physician according to the mode of administration and the weight and response of said patient.

The present invention also relates to the compounds as defined above as such.

The present invention also relates to a compound having the following formula (I-1):

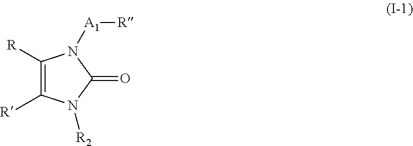

wherein:
   R and R′ are, independently from each other, H or (C₁-C₆)alkyl groups, or form together with the carbon atoms carrying them a (C₆-C₁₀)aryl group;
said aryl group being optionally substituted with one or several substituents, said substituents being in particular selected from the group consisting of:
   halogen;
   (C₁-C₆)alkyl;
   OH;
   (C₁-C₆)alkoxy;
   —NR_{d}Rₑ, R_{d} and Rₑ, independently from each other, being H or a (C₁-C₆)alkyl group;
   aryl;
   heteroaryl;
   halo(C₁-C₆)alkyl group, such as CF₃;
   —C(═O)—NR_{f}R_{g}, R_{f} and R_{g}, independently from each other, being H or a (C₁-C₆)alkyl group; and
   —C(═O)—Rₕ, Rₕ being a (C₁-C₆)alkyl group;
   R₂ is selected from the group consisting of:
      H;
      (C₁-C₆)alkyl group;
      halo(C₁-C₆)alkyl group;
      aryl; and
      heteroaryl;
   A₁ is a linker having the following formula (II):

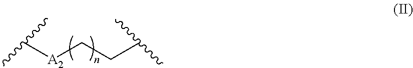

wherein:
   n is an integer varying from 1 to 7; and
   A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
   R″ is a group having the following formula (A-1):

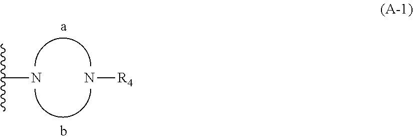

wherein:
   the bonds “a” and “b” form a 4- to 10-membered saturated heterocycloalkyl group with the nitrogen atoms carrying them, said heterocycloalkyl group being optionally substituted for example with at least one substituent selected from (C₁-C₆)alkyl groups, and being selected from the monocyclic groups, bicyclic groups, fused bicycles and spiro-type rings; and
   R₄ is selected from the optionally substituted heteroaryl groups;
or its pharmaceutically acceptable salts, racemates, diastereomers or enantiomers.

According to a preferred embodiment, in formula (I-1), R and R′ form together with the carbon atoms carrying them a (C₆-C₁₀)aryl group, in particular a fused phenyl group.

According to a preferred embodiment, in formula (I-1), R₂ is H.

According to a preferred embodiment, the compounds according to the invention have the following formula (I-2):

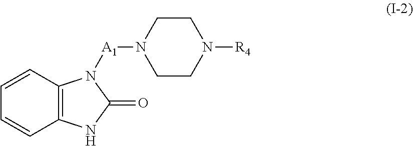

A₁ and R₄ being as defined above.

As compounds having the formula (I-1) or (1-2), the following compounds may be mentioned:

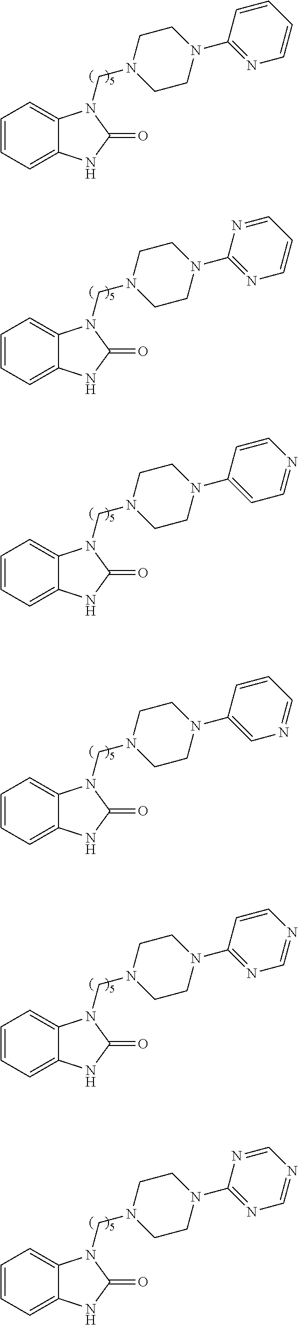

The present invention also relates to a compound having the formula (I-3):

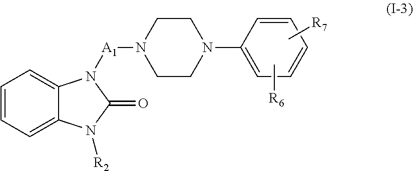

wherein:
   R₂ is selected from the group consisting of:
      H;
      (C₁-C₆)alkyl group;
      halo(C₁-C₆)alkyl group;
      aryl; and
      heteroaryl;
   A₁ is a linker having the following formula (II):

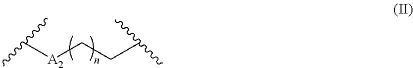

wherein:
   n is an integer varying from 1 to 7; and
   A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
   R₆ is selected from the group consisting of: —OH, (C₁-C₆)alkoxy, (C₁-C₆)alkyl, halogen, and thio(C₁-C₆)alkyl, and
   R₇ is halogen;
or its pharmaceutically acceptable salts, racemates, diastereomers or enantiomers.

Preferably, in formula (I-3), R₂ is H.

As compounds having the formula (I-3), one may cite the following compounds:

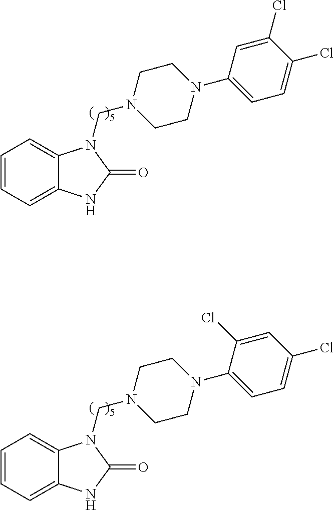

The present invention also relates to a compound having the following formula (I-4):

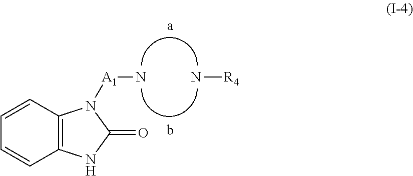

wherein:
   A₁ is a linker having the following formula (II):

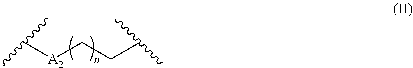

wherein:
   n is an integer varying from 1 to 7; and
   A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
   the bonds “a” and “b” form a 4- to 10-membered saturated heterocycloalkyl group with the nitrogen atoms carrying them, said heterocycloalkyl group being optionally substituted for example with at least one substituent selected from (C₁-C₆)alkyl groups, and being selected from the monocyclic groups, bicyclic groups, fused bicycles and spiro-type rings; and
   R₄ is selected from the optionally substituted (C₆-C₁₀)aryl and heteroaryl groups; or its pharmaceutically acceptable salts, racemates, diastereomers or enantiomers.

Preferably, in formula (I-4), R₄ is an aryl group, and more preferably a phenyl group.

As compounds having the formula (I-4), one may cite the following compounds:

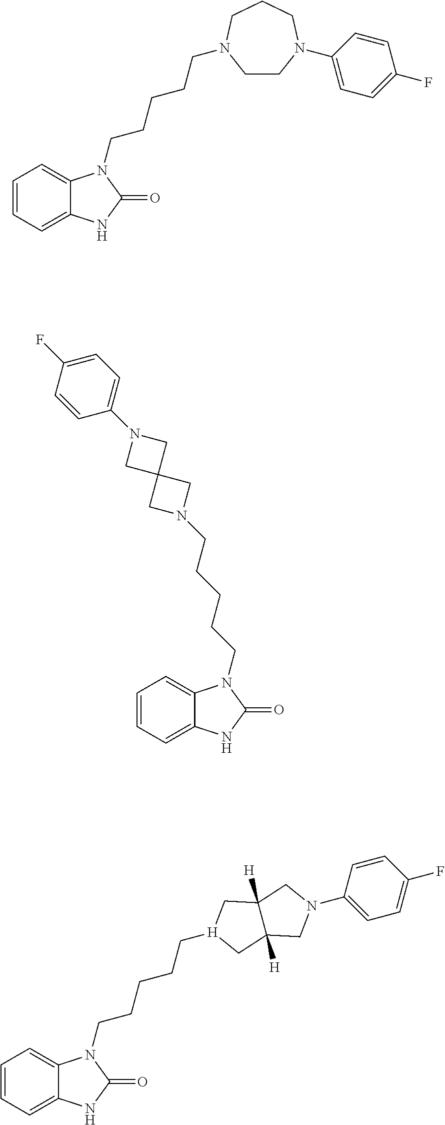

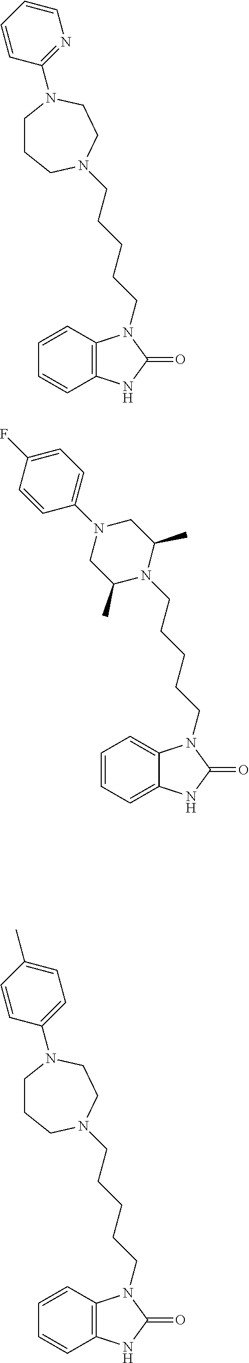 [SMILES: O=c1[nH]c2ccccc2n1CCCCCN1CCCN(c2ccccn2)CC1 ; C[C@@H]1CN(c2ccc(F)cc2)C[C@H](C)N1CCCCCn1c(=O)[nH]c2ccccc21 ; Cc1ccc(N2CCCN(CCCCCn3c(=O)[nH]c4ccccc43)CC2)cc1]

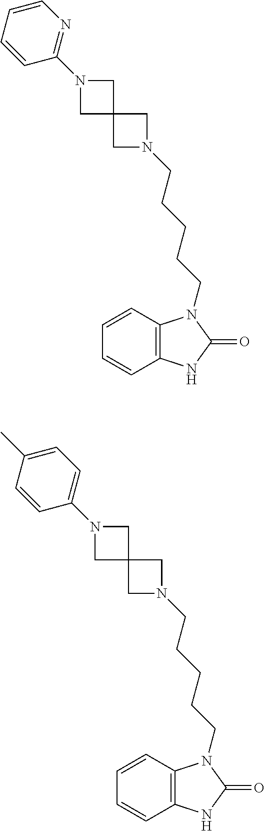 [SMILES: O=c1[nH]c2ccccc2n1CCCCCN1CC2(C1)CN(c1ccccn1)C2 ; Cc1ccc(N2CC3(CN(CCCCCn4c(=O)[nH]c5ccccc54)C3)C2)cc1]

The present invention also relates to a compound having the following formula (I-5):

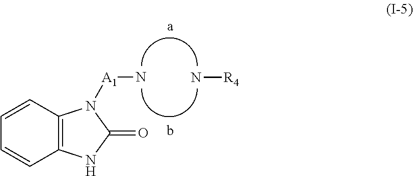

wherein:
   A₁ is a linker comprising from 3 to 10 carbon atoms, wherein possibly at least one carbon atom of A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
   the bonds “a” and “b” form a 4- to 10-membered saturated heterocycloalkyl group with the nitrogen atoms carrying them, said heterocycloalkyl group being optionally substituted for example with at least one substituent selected from (C₁-C₆)alkyl groups, and being selected from the monocyclic groups, bicyclic groups, fused bicycles and spiro-type rings; and
   R₄ is selected from the optionally substituted (C₆-C₁₀)aryl and heteroaryl groups; or its pharmaceutically acceptable salts, racemates, diastereomers or enantiomers.

Preferably, in formula (I-5), R₄ is an aryl group, and more preferably a phenyl group.

Preferably, in formula (I-5), A₁ is a linear or branched alkylene radical comprising from 3 to 10 carbon atoms in its main chain, or is optionally interrupted with one or several heteroatoms, such as —O— as explained above.

As compounds having the formula (I-5), one may cite the following compounds:

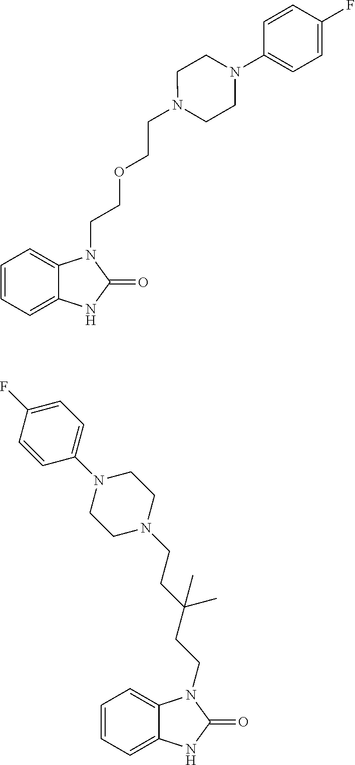 [SMILES: O=c1[nH]c2ccccc2n1CCOCCN1CCN(c2ccc(F)cc2)CC1 ; CC(C)(CCN1CCN(c2ccc(F)cc2)CC1)CCn1c(=O)[nH]c2ccccc21]

The present invention also relates to a compound having the following formula (I-6):

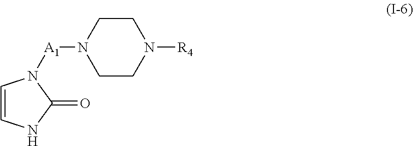

wherein:
   A₁ is a linker having the following formula (II):

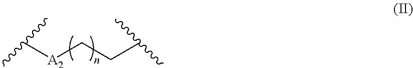

wherein:
   n is an integer varying from 1 to 7; and
   A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
   R₄ is selected from the optionally substituted (C₆-C₁₀)aryl and heteroaryl groups; or its pharmaceutically acceptable salts, racemates, diastereomers or enantiomers.

According to a preferred embodiment, the compounds according to the invention are selected from the following compounds:

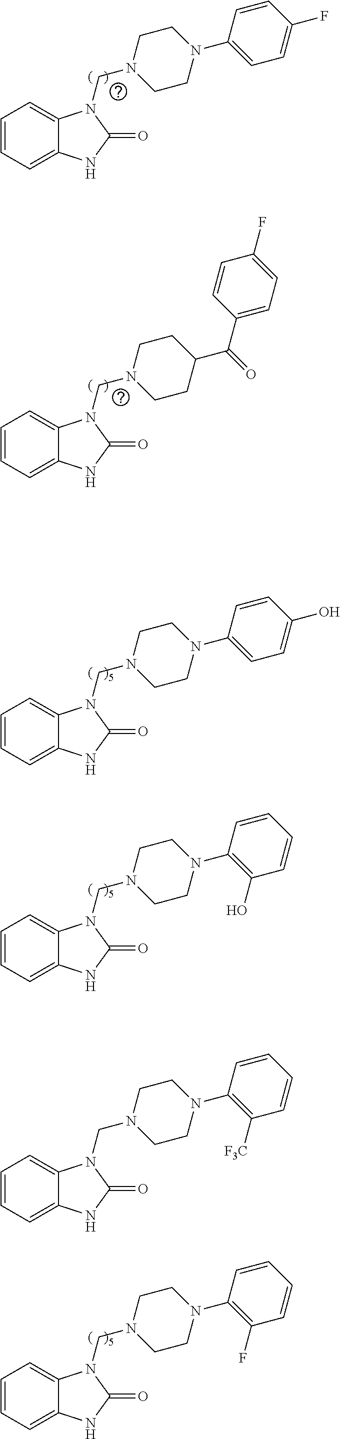

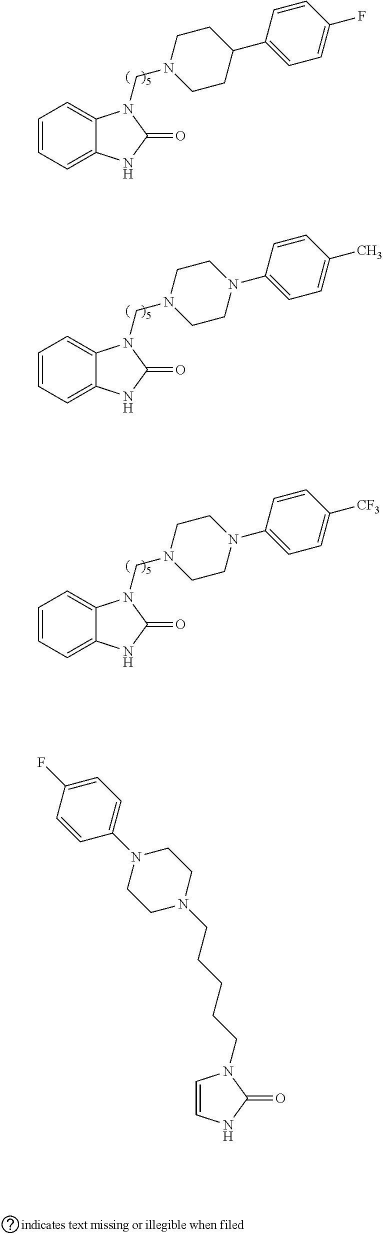

### FIGURES

FIG. 1: Serodolin and MOA-51 act as antagonists/inverse agonists on Gs/cAMP signaling (A) Chemical structure of 5-HT7R ligands. (B) HEK-293 cells stably expressing h5-HT7R were stimulated with 10 nM of 5-CT and increasing concentrations of products for one hour. After cell lysis, cAMP production was quantified by a LANCE cAMP detection kit (Perkin Elmer). (C) HEK-293 cells stably expressing h5-HT₇R were incubated with increasing concentration of SB-269970, Serodolin or MOA-51. Data points represent the means±SEM from three independent experiments performed in triplicate. The EC50 and IC50 for each drug was determined using GraphPad Prism software.

FIG. 2: Serodolin and MOA-51 act as agonists on ERK1/2 signaling. (A, B, C, D, E, F) Time course of activation of ERK1/2 after stimulation of HEK-293 cells stably expressing h5-HT7R with various 5-HT7R ligands used at 10 μM. The cells were stimulated for the indicated periods and assayed for detection of phospho ERK1/2 by western blot analysis. All blots were also probes with anti-ERK1/2 antibody to confirm equal loading. Representative blots of three independent experiments are illustrated. The histogram on the right of each panel represents the results of densitometric analyses of three independent experiments. Data are means±SEM. * p<0.05; ** p<0.01; *** p<0.001 versus non stimulated cells (NS).

FIG. 3: Serodolin-induced ERK phosphorylation is mediated through-5-HT₇R activation. HEK-293 cells stably expressing h5-HT₇R were stimulated with increasing concentrations of 5-CT or Serodolin for 7 minutes. Cells were lysed, and western blot analysis was performed. Representative Western blots from three independent experiments were shown in A-C. Quantification was performed by densitometric analyses from three independent experiments. Analyzed data were plotted versus log concentration for each compounds (A; B) or as bar graph (C) on the right panel. Data are means±SEM. * p<0.001 versus cells without SB269-970 (C).

FIG. 4: Serodolin stimulation induces ERK phosphorylation in neuronal culture. Mixed neuronal cultures from embryos (E15) mice were stimulated with the 5-HT7r agonist 5-CT (10 μM) or with Serodolin (10 μM) or Vehicle (0.1% DMSO diluted in PBS solution) used as a control group, for 7 min, 15 min and 30 min. (A) Co-immunostaining of neuron marker MAP2 (red) with pERK (green) corresponding to the condition with 30 min of stimulation. These images are representative of two independent experiments. (B) pERK fluorescence intensity (AU) from immunofluorescence staining. Results represent mean±SEM of values obtained in two independent experiments (150-200 cells counted per group). ****P<0.0001 ***P<0.001 **P<0.01 *P<0.05. Statistical analysis was done using Tukey's multiple comparison test.

FIG. 5: Serodolin-induced ERK phosphorylation is dependent on Ras and MEK and does not required EGFR or PKA activation. HEK-293 cells stably expressing h5-HT₇R were stimulated with 5-CT (10 μM), Serodolin (10 μM) or Vehicle (0.1% DMSO diluted in PBS solution) for 7 min. Before addition of 5-HT₇R ligands, cells were incubated in the absence or presence of (A) the Ras inhibitor FT1277, (B) the EGFR inhibitor PD153089, (C) the MEK inhibitor PD98059 or (D) the PKA inhibitor H89. Representative blots of three independent experiments are illustrated.

FIG. 6: BRET between 5-HT7-RLuc and Venus-mini G proteins Pharmacological profiling of ligands-mediated G protein recruitment of 5-HT7(b) receptors in HEK 293 cells: Gαs recruitment,) Ga12 recruitment, Gαi recruitment and Gαq recruitment. HEK293 cells were transfected with HA-5HT7(b)-Rluc, and the appropriate BRET acceptors, then incubated with increasing doses of 5-CT, SB269970 or Serodolin (10⁻¹¹ to 10⁻⁵ M). For −mGi, mG12 and mGq cells were also transfected with a receptor described as positively coupled to the G protein: Cells expressing the Adenosine 2 receptor A2R, the Histamine3 receptor H3R or the Ghrelin receptor GHSR were stimulated with adenosine, imetit or ghrelin respectively. Ligand mediated BRET changes are expressed as induced BRET changes which were generated by subtracting at each point the signal obtained on cells incubated with PBS (without ligand).

FIG. 7: Serodolin induced ERK phosphorylation is dependent on c-SRC activation and requires proline-rich regions on 5-HT₇R. (A) HEK-293 cells stably expressing h5-HT₇R were stimulated with increasing concentrations of 5-CT (10M) or Serodolin (10M) for 7 minutes in absence or presence of the potent c-SRC inhibitor PP2 or its inactive analog PP3. Cells were lysed, and western blot analysis was performed. (B) The phosphorylation of ERK1/2 as well as phosphorylation of c-SRC were quantified on the same cell lysates using the AlphaScreen assays. Means±SEM of values from three experiments performed in triplicate. (C) The PXXP motif in the C-terminal tail of 5-HT₇R has been mutated as described in the upper panel. The corresponding constructs encoding HA-tagged 5-HT₇ receptor were transfected in HEK-293 cells as indicated (MutT1, mutT2 and mutT3), then the effect of 5-CT and Serodolin have been evaluated on ERK1/2 and c-SRC activation by Western blot analysis. Representative immunoblots of three independent experiments are illustrated.

FIG. 8: Serodolin-induced ERK phosphorylation is dependent on β-arrestin 2 recruitment. (A) HEK-293 or KO β-arrestin HEK-293 cells were transiently transfected with HA-5-HT7R and stimulated with 5-CT (10 μM), Serodolin (10 μM) or Vehicle (0.1% DMSO diluted in PBS solution) used as a control for 7 min. Representative blots of three independent experiments are illustrated. (B) Quantification of p-ERK and p-c-SRC were performed using Alphascreen technology. Data are means±SEM of values obtained in three independent experiments. (C) HEK-293 cells were transiently transfected with HA-h5-HT7R with β-arrestin2 BRET biosensor (Rluc-Arrestin-YPET), then incubated with increasing doses of 5-CT, or Serodolin (10-11 to 10-5 M). Ligand mediated BRET changes are expressed as induced BRET changes which were generated by subtracting at each point the signal of the cells incubated with PBS (without ligand). Data were fitted using non-linear regression using GraphPad Prism software.

FIG. 9: Analgesic effect of Serodolin in the acetic acid-induced writhing test. In this test, nociception was induced by an intraperitoneally injection (ip) of 0.1 ml/10 g acid acetic solution (10 ml/kg) in peripheral origin. Serodolin at increasing dosage was administrated by oral (po), intravenous (iv) or subcutaneous (sc) route before acid acetic injection (upper panel). Positive control animals were pretreated morphine (3 mg/kg, sc) 10 minutes before acetic acid. Five minutes after i.p. injection of acetic acid the number of writhing was recorded for 10 minutes. Data mean±SEM of values obtained from a representative experiment (n=10 animals/group). ****p<0.0001 ***p<0.001 **p<0.01 *p<0.05 as compared with the control group (Vh).

FIG. 10: Dose-response and kinetic antinociceptive effect of the 5-HT7R agonist Serodolin on tail immersion test. (A) Experimental protocol summary used. (B) Mice were subcutaneously injected with two different doses of the 5-HT7 receptor agonist Serodolin, 1 mg/kg or 5 mg/kg and 10 min later their tail extremity was immersed in water heated to 50 degrees. (C) Serodolin (5 mg/kg) antinociceptive effect was compared with E55888 (5 mg/kg), the agonist reference of 5-HT7 receptor associated with a kinetic study. The effect of injections (Serodolin or E55888) was evaluated at TO, 30 min and 60 min corresponding to 10, 40 and 70 min after compound injections. (D) Experimental protocol summary used to test the effect of SB269970. Mice were intraperitoneally injected (+) or not (−) with the 5-HT₇R antagonist SB269970, 10 min before agonist tail subcutaneous injections (E55888 or Serodolin at 5 mg/kg). Their tail extremity was immersed in water heated to 50 degrees at TO (E) or 30 min later (F) corresponding to 10 and 40 min after agonist injections. Data are means±SEM of values obtained in two independent experiments (n=10 per group). ****p<0.0001 vs. mice treated with vehicle without SB269970, **p<0.01. Statistical analysis was done using Tukey's multiple comparison test.

FIG. 11: Antinociceptive effect of the 5-HT₇R agonist Serodolin on CFA induced mechanical hypersensitivity. Mechanical hypersensitivity was evaluated after CFA intraplantar injection by using the Von Frey test. (A) Experimental protocol summary used. Mechanical hypersensitivity was performed 24 hrs later (pre-treatment) CFA intraplantar injection into the left hind paw (ipsilateral paw). (B) Mice were intraperitoneally injected (+) or not (−) with the 5-HT₇R antagonist SB269970, 20 min before agonist subcutaneous injections (E55888 or Serodolin at 5 mg/kg). The ligand effects (E55888 or Serodolin) were evaluated 30 min and 24 hrs after in the ipsilateral paw. Data are means±SEM of values obtained in 2 independent experiments (n=8 to 10 per group). ****p<0.0001 ***p<0.001 **p<0.01 *p<0.05. Statistical analysis was done using Kruskal Wallis test.

FIG. 12: Evaluation of the therapeutic potential of Serodolin in EAE model. A/Serodolin has been administrated for 10 days from day 8 after immunization. After 18 days post-immunization, myelin staining and cell infiltration have been evaluated in 3 groups of mice (non-immunized NI, Vehicle-treated mice or Serodolin-treated mice. B/Immunolabeling of astrocytes (GFAP) and microglia (Iba1) performed in all groups. Quantifications used Image J software. **p<0.01 *p<0.05.

FIG. 13: Evaluation of the effect of Serodolin on body temperature.

FIG. 14: Testing of 8 molecules for radioligand binding competition activity on recombinant human 5-HT1A, 5-HT2A, 5-HT2Cedited, 5-HT6, 5-HT7 and D2(long) receptors using filtration binding assays.

% Binding of molecules (Serodolin (AIC01), MOA51, JLB009, JLB012, JLB016, JLB018, JLB060, JLB094) with R5-HT7

FIG. 15: JLB060 induced ERK phosphorylation JLB060 act as agonists on ERK1/2 signaling. Time course of activation of ERK1/2 after stimulation of HEK-293 cells stably expressing h5-HT7R with JLB060, a 5-HT7R ligand used at 10 μM. The cells were stimulated for the indicated periods and assayed for detection of phospho ERK1/2 by western blot analysis. The blot was probes with anti-GAPDH antibody to confirm equal loading.

FIG. 16: Analgesic effect of MOA51 in the acetic acid-induced writhing test. In this test, nociception was induced by an intraperitoneally injection (ip) of 0.1 ml/10 g acid acetic solution (10 ml/kg) in peripheral origin. MOA51 at increasing dosage was administrated by oral (po), intravenous (iv) or subcutaneous (sc) route before acid acetic injection (upper panel). Positive control animals were pretreated morphine (3 mg/kg, sc) 10 minutes before acetic acid. Five minutes after i.p.injection of acetic acid the number of writhing was recorded for 10 minutes. Data mean±SEM of values obtained from a representative experiment (n=10 animals/group). **p<0.01 as compared with the control group (Vh).

FIG. 17: Evaluation of the effect of MOA51 on body temperature. Results expressed in ° C. Vehicle: 20% (v/v) DMSO/5% (v/v) Tween 80 in saline **:P<0.01, when compared with the control group: analysis of variance for repeated measurements with DunnettOs test if P<0.05.

FIG. 18: Effect of a single administration of Serodolin (AIC01) or MOA51 in the formalin test in rats. Effect of a single subcutaneous administration of Serodolin (AIC01) or MOA51 in the formalin test in rats. In this test, Sprague-Dawley male rats received unilateral injection of a 2.5% formalin solution (50 μl) into the plantar aspect of the hindpaw on testing day (i.e. D0). Control group received Vehicle (20% DMSO/5% Tween 80/NaCl). Serodolin was subcutaneously (sc) administrated at 10 mg/kg and MOA51 at 1 mg/kg. Positive control animals were treated with morphine (3 mg/kg, sc). Paw licking time was measured. Results are expressed as mean±s.e.m. Percentage are expressed as decreased as compared to the vehicle-treated group and represented as figure (B). ***: p<0.001 as compared to the vehicle-treated group, Bonferroni's test after significant Two-way Repeated Measures ANOVA. NS: Non-significant.

FIG. 19: Antalgic effect of repeated administration of MOA51 and AIC01 compounds on Spared Nerve Injury (SNI) neuropathic pain mice model. Analgesic effect of Pregabalin (Positive control mice) and of Serodolin (AIC01) and MOA51 compounds on mechanical allodynia in SNI neuropathic pain mice model. Pregabalin, vehicle or compounds solutions are subcutaneously administrated for 8 consecutive days.
(A) Mechanical response threshold after the first administration (D+10). Serodolin was subcutaneously (sc) administrated at 0.5 mg/kg and MOA51 at 5 mg/kg to C57BL/6 mice. Positive control animals were treated with Pregabalin (5 mg/kg, sc). Statistical differences are indicated compared to vehicle group (Two-way RM ANOVA followed by Bonferroni post-hoc test: ***p<0.001, ** p<0.01, * p<0.05). See also annex for statistical analyses reports.
(B) Mechanical response threshold after the last administration (D+18). Serodolin was subcutaneously (sc) administrated at 0.5 mg/kg and MOA51 at 5 mg/kg to C57BL/6 mice. Positive control animals were treated with Pregabalin (5 mg/kg, sc). Statistical differences are indicated compared to vehicle group (Two-way RM ANOVA followed by Bonferroni post-hoc test: ***p<0.001, ** p<0.01, * p<0.05). See also annex for statistical analyses reports. (C) Repetitive administration of Pregabalin, MOA51, Serodolin (AICO1) and vehicle in SNI neuropathic pain model. (Two-way RM ANOVA followed by Bonferroni post-hoc test: ***p<0.001, ** p<0.01, * p<0.05)
(D) AUC of time-course for indicated days for repetitive administration of Pregabalin, MOA51, Serodolin (AIC01) and vehicle in SNI neuropathic pain model. (Two-way RM ANOVA followed by Bonferroni post-hoc test: ***p<0.001, ** p<0.01, * p<0.05)

FIG. 20: Pharmacokinetic study of E55888 and Serodolin. Concentration-time profile of E55888 (reference agonist) and Serodolin in plasma (A) and brain (B) following subcutaneous injection of a unique dose 5 mg/kg of each compound in C57BL/6 mice. Data represent the mean concentration±SEM of n=4 each time point.

### EXAMPLES

### Material and Methods

### Drugs, Antibodies, Reagents, and Medium

Coelenterazine was from Interchim (Montlugon, France). The protease inhibitor cocktail was from Roche (Mannheim, Germany). PP2, PP3 and PTX were from Callbiochem. PVDF membrane and CL-X film were from GE Healthcare (Chalfont St. Giles, United Kingdom). The Pierce supersignal extended Dura chemiluminescent substrates and medium for cell culture were from Thermo Fisher Scientific Inc (Rockford, Illinois, USA). The rabbit anti-mouse (816720) and goat anti-rabbit (656120), IgG HRP-linked whole antibodies were from Life technologies (Carlsbad, California, USA). All other reagents and culture media were from Sigma Aldrich (St Louis, Missouri, USA). KO arrestin cells line were kindly provided by Dr Asuka Inoue (Tohoku University, Japan). The GHSR fused to Renilla lucifersae were kindly provided by Janques Pantel (UMRS 1124, Paris, France). The N-terminal 3×HA tagged human 5-HT_{7b}R were obtained from the cDNA Resource Center (www.cdna.org)

### Plasmid Constructs

In order to generate the human 5-HT₇R construct conjugated at its C-terminus Renilla Lucifersae (5-HT₇-RLuc) used for BRET experiments, Nhel and EcoRI sites were inserted upstream and downstream respectively the ORF of the human 5-HT₇R in the pRLuc-N1 vector, that we have previously obtained (Cobret, L., De Tauzia, M. L., Ferent, J., Traiffort, E., Henaoui, I., Godin, F., Kellenberger, E., Rognan, D., Pantel, J., Benedetti, H., and Morisset-Lopez, S. (2015) Targeting the cis-dimerization of LINGO-1 with low MW compounds affects its downstream signalling. Br J Pharmacol 172, 841-856). The 5-HT₇R-Nhel forward primer 5′-CGACGTGCTAGCGCCACCATGTACCCATACGATGTTCCAGAT-3′ (SEQ ID NO:1) and the 5-HT₇R-EcoRI reverse primer 5′-CTGAGCGAATTCGTGATGAATCA TGACCTTTTTTTCTACA G-3′ (SEQ ID NO:2) were used for that purpose. The fragments obtained from BamH1 restriction of the polymerase chain reaction (PCR) product were ligated into the pRLucN1 vector linearized by digestion with Nhel and EcoRI. Mutations in the PXXP motif were performed by site-directed mutagenesis. All sequence obtained were verified by direct DNA sequencing (MWG Eurofins, Germany and Cogenics, France).

### Cell Cultures and Transfections

HEK293 cells and HEK293 cells stably expressing 5-HT_{7b}R were grown in Dulbecco's modified Eagle medium (DMEM) supplemented with 10% (vol/vol) dialyzed fetal calf serum, 100 U/ml penicillin, 0.1 mg/ml streptomycin. KO arrestin cells line were kindly provided by Dr Asuka Inoue.

Cerebral cortex from embryos (E15) mice were collected and mechanically dissociated in 1 mL of HBSS (—Ca²⁺) (Sigma) with HEPES (H3375, Sigma). After addition of 1 mL of HBSS (+Ca⁺²) with HEPES and 1 mL of decomplemented fetal calf serum (FCS) (12133C, Sigma), samples were centrifuged (1000 rpm, 30 min) to collect cells in 1 mL of Neurobasal (GIBCO, Thermo Fisher Scientific). Neuronal differentiation was performed in 24-well plates, during 7 days with 3 medium changes per week.

For BRET experiments cells were transfected with the calcium phosphate precipitation method. cAMP determinations, cells were transiently transfected with 10 μg of plasmid/100-mm dish with Lipofectamine 2000 (Invitrogen) and Opti-MEM (Gibco), according to manufacter's recommendations. Experiments were performed 24 h to 48 h after transfection. siRNA were transfected with Lipofectamin 2000 (Invitrogen) in HEK-293 cells.

cAMP Accumulation and Functional Assays

cAMP accumulation was measured with a LANCE™ cAMP detection kit (Perkin-Elmer Life Sciences, Boston, MA, USA). 12 h before experiment cells were starved. Forty-eight h after transfection, cells were harvested in Hank's balanced salt solution (HBSS) containing 5 mM HEPES, 1 mM isobutylmethylxanthine (IBMX) and 0.1% BSA, PH 7.4. After centrifugation (1000×g, 5 min), cells were resuspended in the same buffer (2×10⁶ per ml). The ALEXA™ fluor 647 anti-cAMP antibody solution (1 μl) was added to the cell suspension (100 μl) and 5 μl aliquots of the mixture were dispensed in white 384-well microtiter plates (Optiplate, Perkin Elmer). The cells were then stimulated with different drugs. After 1 h incubation at room temperature in the dark, lysis buffer (0.35% Triton X-100, 10 mM CaCl₂, 50 mM HEPES) containing LANCE EU-W8044 labeled streptavidin and biotinylated-cAMP was added to the cells (10 μL per well). After 2 h incubation at room temperature in the dark, the plates were read on a VictorV™ microplate reader (Perkin-Elmer Life Sciences). Concentration/response curves were analyzed using Prism 4 software.

### Flow Cytometry

Cells were transfected with the different mutants of the receptor fused to HA tag and grown until 70% confluence and washed using complete PBS (PBS with 1 mM of CaCl₂ and 0.5 mM of MgCI₂). Cells were then detached and incubate with anti-hemagglutinin (HA) from Roche Diagnostics (Meylan, France) for 60 min, followed by an additional incubation with Goat Anti-Rat (FITC) antibody (ab6840) for 60 min. Isotypic controls were done in parallel by incubation with each corresponding immunoglobulin isotype. After washing, stained cells were analyzed by flow cytometry using BD LSR cytometer (BD Biosciences) and results from 10,000 cells were analyzed by Cell Quest Pro software. Results are presented as the difference in fluorescence relative intensity between the cells labeled with the antibody versus the cells labeled with the corresponding isotype, and by the overlay histograms displaying the isotypic control and the antibody labeling, for one representative experiment out of two.

### SDS-PAGE and Western Blot

Treated cells were washed twice with cold PBS and lysed on ice for 30 min in lysis buffer containing 50 mM Tris pH 7.5, 150 mM NaCl, 10 mM EDTA, 1% Triton X-100 and the protease inhibitor cocktail. Cell lysates were centrifuged at 10,000×g for 10 min. Supernatant was then solubilized in Laemmli buffer with 0.1% β-mercaptoethanol. Samples were resolved by electrophoresis on 10% SDS-PAGE, and transferred electrophoretically to polyvinylidene fluoride (PVDF) membranes (GE Healthcare Life Sciences). Nitrocellulose membranes were washed in Tris-buffered saline (TBS; pH 7.4) containing 0.1% Tween-20 (TBS-T; 0.1%) and blocked with 5% (w/v) dry milk TBS-T 0.1% for 30 min. Blots were probed with Blots were probed with anti-arrestin, anti-Phospho-ERK or anti-ERK antibody (1:2000), anti Phospho-SRC, anti SRC antibody, anti-HA or anti-GFP or anti-actin antibodies. Horseradish-peroxidase-conjugated goat anti-rabbit, anti-mouse or anti-rat antibodies (1:33,000) were used as secondary antibodies. Immunoreactive bands were detected using the Dura detection kit. Protein quantification on blots was performed using Quantity One software (Biorad).

### AlphaScreen Assays

The AlphaScreen SureFire phospho-ERK and phosphor-c-SRC assays (PerkinElmer) were used to quantify pERK1/2 and pc-SRC from HEK-293 cell lysates according to the manufacturer's instructions.

### BRET Analyses

To evaluate Gαs, Gαq, Gαi recruitment, cells were transiently transfected with 5-HT₇R(b) C—terminally fused with donor Rluc (5-HT₇R-Rluc) and acceptor NESVenus. mGs, or NES-Venus-mGsq or NES-Venus-mGsi (kindly provided by Pr. N. A. Lambert, Augusta University, Augusta, USA) (Ayoub, M. A., Landomiel, F., Gallay, N., Jegot, G., Poupon, A., Crepieux, P., and Reiter, E. (2015) Assessing Gonadotropin Receptor Function by Resonance Energy Transfer-Based Assays. Front Endocrinol (Lausanne) 6, 130; Wan, Q., Okashah, N., Inoue, A., Nehme, R., Carpenter, B., Tate, C. G., and Lambert, N. A. (2018) Mini G protein probes for active G protein-coupled receptors (GPCRs) in live cells. J Biol Chem 293, 7466-7473). For the assessment of β-arrestin 2 recruitment, HEK293 cells were transiently co-transfected with plasmids coding for 5-HT₇R-Rluc and for Rluc-β-arrestin 2 yPET (kindly provided by Dr. M. G. Scott, Cochin Institute, Paris, France). Forty-eight hours after transfection BRET measurements were immediately performed upon addition of a rising concentrations of different ligands and 5 μM of coelenterazine H. Signals were recorded for 30 minutes in a Mithras LB 940 Multireader (Berthold, Bad Widbad, Germany), which allows the sequential integration of luminescence signals detected with two filter settings (RLuc filter, 485±10 nm; YFP filter, 530±12 nm). Emission signals at 530 nm were divided by emission signals at 485 nm. The results were expressed as induced-BRET change corresponding to the difference between the BRET ratio observed in control conditions (without ligands) and those obtained after addition of 5-HT7 ligands. The results are shown as mean±SEM from 3 independent experiments. Data were plotted and analysed using GraphPad Prism 4 software for Windows (GraphPad Software Inc, San Diego, CA, USA). For normalization the value of all replicates were divided by the mean of the agonist 5-CT induced maximal responses and multiplied by 100 for any given read out. Concentration-response curves were fitted by nonlinear regression and saturation curves by a hyperbolic one-binding site equation. The method provided estimates for EC50 values and corresponding SEM.

### Mice

Wild-type C57BL/6 mice were purchased from Janvier Labs (Le Genest Saint Isle, France). For experiments, male animals (8-10 week-old) were housed in our animal unit and kept under controlled conditions of bright cycle (12/12 h), temperature (20-22° C.) and humidity (50%). Ligands were solubilized in 20% DMSO, 5% Tween 80 diluted in PBS solution for injection in mice. All animal protocols were carried out accordingly with the French Government animal experiment regulations and were approved by the local ethics committee for animal experimentation in Orleans (CEO3) (APAFIS #24374-2020010614026010 v9 and APAFIS #2018070915377687).

Autoimmune experimental encephalomyelitis (EAE) model. For EAE model, the pathology was induced by subcutaneous injection of an emulsion of MOG35-55 peptide in complete Freund's adjuvant as previously described (Terry, Ifergan, & Miller, 2016). The mice were scored blindly once a day starting at Day 7 postimmunization until Day 30 according to the following scale: 0.0=no obvious changes in motor function; 0.5=tip of tail is limp; 1.0=limp tail; 1.5=limp tail and hind leg inhibition; 2.0=limp tail and weakness of hind legs or signs of head tilting; 2.5=limp tail and dragging of hind legs or signs of head tilting; 3.0=limp tail and complete paralysis of hind legs or limb tail with paralysis of one front and one hind leg; 3.5=limp tail and complete paralysis of hind legs and animal unable to right itself when placed on its side; 4.0=limb tail, complete hind leg, and partial front leg paralysis with minimal moving and feeding. Drugs were administered at the onset of clinical symptoms (Day 8) until Day 18 after immunization. Serodolin (1 mg/kg, ip) or vehicle was daily administered. Mice were given Ketamine/Xylasine anaesthesia and then intracardiacally perfused first with PBS EDTA for 20-30 min and then with PFA (paraformaldehyde) 4% for 20-30 min. Spinal cords were removed and incubated first in PFA 4% for 48-72 h and then in sucrose 30% Organ was included in TFM (Tissue freezing media) and snap frozen using isopentane and dry ice. Spinal cord were cut on 14 μm thick sections using Leica CM3050 S Research Cryostat for immunohistochemistry experiments.

### Immunocytochemistry

Stimulated neuronal culture were fixed with 4% paraformaldehyde in PBS during 10 min and then washed with PBS. Cells were saturated with 0.3% Triton X-100 in 1% BSA in TBS-FCS 10% for 1 hr, followed by three washes in TBS and incubated overnight with rabbit anti-pERK (9101 1/200, Cell Signaling) and mouse anti-MAP2 (119942 1/250, Sigma), anti-GFAP (G61711/250, Sigma) in TBS with 1% BSA, 10% FCS and 0.3% Triton X-100. Then, cells were washed three times with TBS and incubated with the corresponding secondary antibodies, sheep anti-rabbit IgG FITC (F7512 1/500, Sigma) or goat anti-mouse IgG TRITC (T7657 1/100, Sigma) for 2 hours in wet and dark room. After three washes, cells were stained with bisBenzimide H33258 (B1155, Sigma) for 10 min, washed and mounted onto microscope slides with Fluoromount-G® (0100-01, SouthernBiotech). The co-stainings were observed using an inverted Zeiss CELL OBSERVER 27 microscope with a 40× EC PLAN NEOFLUAR 40/0.75 NA objective (Carl Zeiss Co. Ltd., Jena, Germany). Images were processed with the Zen software analyzed with Image J.

### Nociception Assessments

Writhing tests: Mice (25-35 g). Groups of mice (n=10) received by oral, subcutaneous or intravenous route Serodolin at different doses (0.1-10 mg/kg) one hour before intraperitoneally injection of 1% acetic acid in a volume of 10 ml/kg. Control group received vehicle (10 ml/kg, solution of 20% DMSO and 5% tween 80). The test was carried out 5 minutes later after acid acetic injection. The characteristic writhing responses have been observed individually and counted for 10 minutes.

Von Frey filament test. Before inducing peripheral inflammation, each animal was tested on the left hind paw with Von Frey Filament to determine its basal sensibility level. Then, peripheral inflammation was induced by intraplantar injection of Complete Freund's Adjuvant (CFA-10 μL) (F5881, Sigma) in the left hind paw (ipsilateral paw) and mechanical allodynia was measure (pre-treatment). Mice were divided into two groups, one with E55888 (5 mg/kg) and the other with Serodolin (5 mg/kg) subcutaneous injections in the ipsilateral paw. Both in absence (−) or in presence (+) of the 5-HT₇R antagonist SB269960 (5 mg/kg), intraperitoneally, 20 min before the agonist injections (8-10 mice per group). The ligands effects on mechanical allodynia were analyzed 30 min and 24 hrs after (post-treatment) and the results were reported at 100% allodynia of each mouse.

Tail immersion test. Nociception was assessed with the tail immersion test, 10 min and 40 min after E55888 (5 mg/kg) or Serodolin (5 mg/kg) tail subcutaneous injections, in the water heated to 50° C. Vehicle (20% DMSO, 5% Tween 80 diluted in PBS solution) was used as a control group. These different groups were intraperitoneally injected (+) or not (−) with the 5-HT₇R antagonist SB269960 (5 mg/kg) 10 min before the ligand injection. The tail withdrawal latency (s) was measured for each animal.

### Radioligand Binding Assay

Radioligand Binding experiments were conducted with Epics Therapeutics membrane preparations. Receptor accession numbers, cellular background and reference compounds are shown in this table.

| | Accession | Cell | Reference | Reference |
|---|---|---|---|---|
| Receptor | Number | line | agonist | competitor |
| 5-HT1A | NP_000515.2 | CHO-K1 | [³H]- | 5-HT |
| | | | 8-OH-DPAT | |
| 5-HT2A | NP_000612.1 | CHO-K1 | [³H]-DOI | 5-HT |
| 5-HT2Cedited | AAF35842.1 | CHO-K1 | [³H]-DOI | 5-HT |
| 5-HT6 | NP_000862.1 | CHO-K1 | [³H]-LSD | Mianserine |
| 5-HT7a | NP_000863.1 | CHO-K1 | [³H]-LSD | 5-CT |
| D2(long) | AAB26819.1 | CHO-K1 | [³H]- | Risperidone |
| | | | Spiperone | |

The new compounds have been tested by radioligand binding competition activity at the human 5-HT1A (FAST-0500B), 5-HT2A (FAST-0505B), 5-HT2Cedited (FAST-0507B), 5-HT6 (FAST-0509B), 5-HT7a (FAST-05111B) and D2(long) (FAST-0101B) receptors at seven (7) concentrations, in duplicate. On each day of experimentation, reference compounds were tested at several concentrations in duplicate (n=2) to obtain a dose-response curve and an estimated EC50/IC50 value. Reference values thus obtained for the test were compared to historical values obtained from the same receptor and used to validate the experimental session. For replicate determinations, the maximum variability tolerated in the test was of +/−20% around the average of the replicates.

Dose-response data from test compounds were analyzed with XLfit (IDBS) software using nonlinear regression applied to a sigmoidal dose-response model and the following equation:
XL Fit fit Model 203: 4 Parameter Logistic Model
A: Bottom
B: TOP
C: LogEC50
D: Hill

fit=(A+((B−A)/(1+(((10\hat{ }C)/x)\hat{ }D))))

inv=((10\hat{ }C)/((((B−A)/(y−A))−1)\hat{ }(1/D)))

res=(y−fit)

### SDS-PAGE and Western Blot

Treated cells were washed twice with cold PBS and lysed on ice for 30 min in lysis buffer containing 50 mM Tris pH 7.5, 150 mM NaCl, 10 mM EDTA, 1% Triton X-100 and the protease inhibitor cocktail. Cell lysates were centrifuged at 10,000×g for 10 min. Supernatant was then solubilized in Laemmli buffer with 0.1% β-mercaptoethanol. Samples were resolved by electrophoresis on 12% SDS-PAGE, and transferred electrophoretically to polyvinylidene fluoride (PVDF) membranes (GE Healthcare Life Sciences). Nitrocellulose membranes were washed in Tris-buffered saline (TBS; pH 7.4) containing 0.1% Tween-20 (TBS-T; 0.1%) and blocked with 5% (w/v) dry milk TBS-T 0.1% for 30 min. Blots were probed with Blots were probed with anti-Phospho-ERK (1:2000) or anti-GAPDH antibody (1:5000). Horseradish-peroxidase-conjugated goat anti-rabbit, anti-mouse or anti-rat antibodies (1:33,000) were used as secondary antibodies. Immunoreactive bands were detected using the Dura detection kit. Protein quantification on blots was performed using Quantity One software (Biorad).

### Formalin Induced Inflammatory Pain Model in Rats

Sprague-Dawley male rats (n=10 rats per group) received unilateral injection of a 2.5% formalin solution (50 μl) into the plantar aspect of the hindpaw on testing day (i.e. D0). Experience was separated into 4 groups of animals. Formalin animals+vehicle-treated group; p.o—Formalin animals+Sponsor's Serodolin (10 mg/kg), s.c.—Formalin animals+Sponsor's MOA51 (1 mg/kg), s.c.—Formalin animals+internal validator-treated group (Morphine at 4 mg/Kg, s.c.). Single subcutaneous administration of the Vehicle and Sponsor's Compound 30 min (or other timing depending on Sponsor's design) before formalin injection on testing day (i.e. D0)/Subcutaneous administration of Morphine 30 min before formalin injection on testing day (i.e. D0). Hindpaw licking time recorded in consecutive 5 minutes periods from 0 to 5 minutes (early phase) and 17 to 27 minutes (late phase) after formalin injection.

### Spared Nerve Injury (SNI) Mice Neuropathic Pain Model

Pregabalin (Tocris) was diluted at 0.3 mg/mL in PBS (Gibco, ref 14190-094). MOA51 and Serodolin (AIC01) compounds were diluted in NDT solution (NaCl 0.9%—DMSO 20%—Tween80 5%). MOA51 was resuspended at 50 μg/mL and was administrated at a ratio of 100 μl per 10 g (dose of 0.5 mg/kg). AIC01 was resuspended at 0.5 mg/mL and administrated at a ratio of 100 μl per 10 g (dose of 5 mg/kg).

Experiment starts with 8 weeks old male C57B16J mice (from Charles River). Study was performed on 40 mice (2 mice were excluded during the study due to health guidelines) divided in 4 randomized groups.
Group A: s.c. injection at 5 mg/kg of pregabalin.
Group B: s.c. injection at 0.5 mg/kg of MOA51.
Group C: s.c. injection at 5 mg/kg of Serodolin (AIC01).
Group D: s.c. injection of NDT solution (referred as Vehicle hereafter).

Compounds solutions, pregabalin and vehicle were subcutaneously administrated (100 μl/10 g) for 9 consecutive days, starting 10 days after surgery. Ligature and transection of the common peroneal and tibial distal branches of the sciatic nerve was made leaving the sural branch intact. 7 days post-surgery, a decrease of threshold response to Von Frey filaments of ipsilateral hind-paw was observed corresponding to neuropathic pain apparition (mechanical allodynia). Mechanical threshold response of mice were measured with calibrated Von Frey filaments using the up/down method. Experimenter was blind to mice treatment. Measures are performed as follow: one baseline measure before surgery, one measure at D+10, D+12, D+14, D+16 and D+18 before drug's administration, and 1 h, 2 h post-drug administration. A supplementary measure 4 h post-drug administration was performed at D+10 and D+18.

Statistical analysis was performed using SigmaPlot 12.5 software. Two-way RM ANOVA (followed by Bonferroni post-hoc test) was used to analysed time course response at D+10 and D+18. Area under the curve was determined with the 1 h and 2 h post-drug administration measures to investigated tolerance of repeated administration of compounds

### Pharmacokinetic Study in Mice

The pharmacokinetic study was undertaken to evaluate and compare the quantity of Serodolin and E55888 in plasma and brain samples from C57BL/6 mice (Janvier, Le Genest France) at several time points. Seven weeks old C57BL/6 mice (n=4 animals per group) received by subcutaneous injection a unique dose (5 mg/kg) of Serodolin or E55888 (U103013S, Achemblock). Control group received vehicle (solution of 20% DMSO and 5% Tween 80 diluted in NaCl). Mice were sacrificed 15 min, 30 min, 60 min, 120 min, 240 min or 480 min after injection. Blank vehicle mouse brains homogenates and plasma were used to established standard curves of Serodolin or E55888. A reference agonist of the 5-HT7R, 5-CT (0458, Tocris), was chosen as internal standard and diluted at 0.025 mg/kg in acetonitrile. Study samples, brains homogenates and plasma, were prepared for protein precipitation by adding 85 μL of acetonitrile+5-CT to 20 μL of samples. LC-HRMS analysis for PK studies were performed on a maXis Q-TOF mass spectrometer (Bruker, Bremen, Germany) coupled to an U3000 RSLC UHPLC system (Dionex, Germering, Germany). Separation was obtained using an Acquity UPLC BEH C18 column (2.1×50 mm; 1.7 μm) (Waters, Saint-Quentin-en-Yvelines, France) thermostated at 40° C. with a gradient of water (solvent A) and acetonitrile (solvent B), both acidified with 0.1% formic acid at 500 μL/min. The gradient was as follows: 2% B from 0 to 0.1 min, a linear gradient up to 98% B at 2.4 min, kept to 3.5 min and reconditioning of the column at 2% B from 3.6 to 5.8 min. The samples were randomized prior analysis; 1.25 μL were injected for plasma and 8 μL for brains. Mass spectra were recorded in the 50-1650 m/z range at a frequency of 4 Hz with positive electrospray ionization. Area were integrated from extracted ion chromatograms (EIC) of [M+H]+ ions using QuantAnalysis 4.4 software (Bruker) with a tolerance of ±0.005 u.

### Statistical Analysis

All results are shown as mean±SEM. For in vivo experiments, statistical analysis was performed using nonparametric Kruskal-Wallis test followed by Dunn post test or a two-way Anova with Tukey post hoc test. The quantification of pERK fluorescence intensity on neuronal culture was analyzed using a two-way Anova with Tukey post hoc test.

### Results

Though several 5-HT₇R antagonists have been successfully developed during the past two decades, agonists often suffer from their lack of specificity or their poor ability to cross the BBB to be used in clinical development. We previously identified a new class of potent 5-HT₇R antagonists derived from pharmacomodulation studies (Deau et al., as mentioned above).

Identification of Serodolin, a Biased Ligand with Differential Effects on AC and ERK Pathways

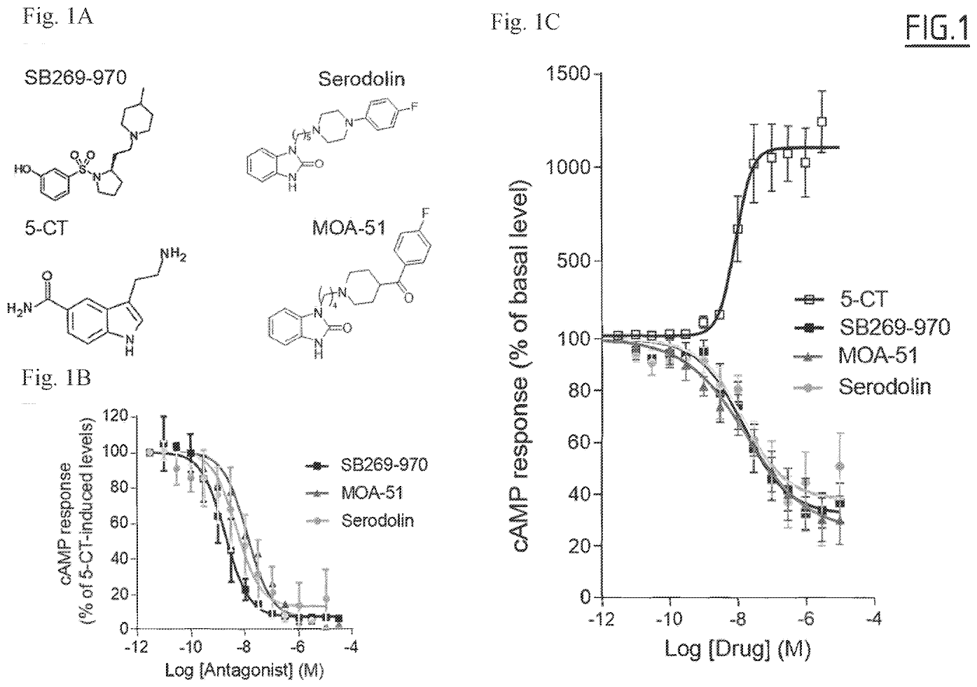

HEK-293 fibroblast stably expressing 5-HT₇ receptor were used to compare the effect of different 5-HT₇R ligands on the classical Gαs-mediated activation of AC pathway. It was decided to evaluate the lead compounds from two series of ligands, Serodolin and MOA-51 (FIG. 1A). As expected, 5-carboxamidotryptamine (5-CT), the full 5-HT receptor agonist, induced a concentration-dependent accumulation of cAMP in HEK-293 cells expressing 5-HT₇R (FIG. 1C). In agreement with previous study, the inventors showed that Serodolin and MOA-51 behave as potent antagonist by decreasing the 5-CT induced cAMP accumulation (IC₅₀=5±2 nM and 12±6 nM, respectively) and in the same manner as the reference antagonist SB269970 (IC₅₀=2±1 nM) (FIG. 1B). Interestingly, when tested alone, Serodolin and MOA-51, like SB269970, produced inverse agonist effects on cAMP response, inhibiting cAMP production by around 75% with high potency (IC50=14 6, 17±6 nM respectively) (FIG. 1C).

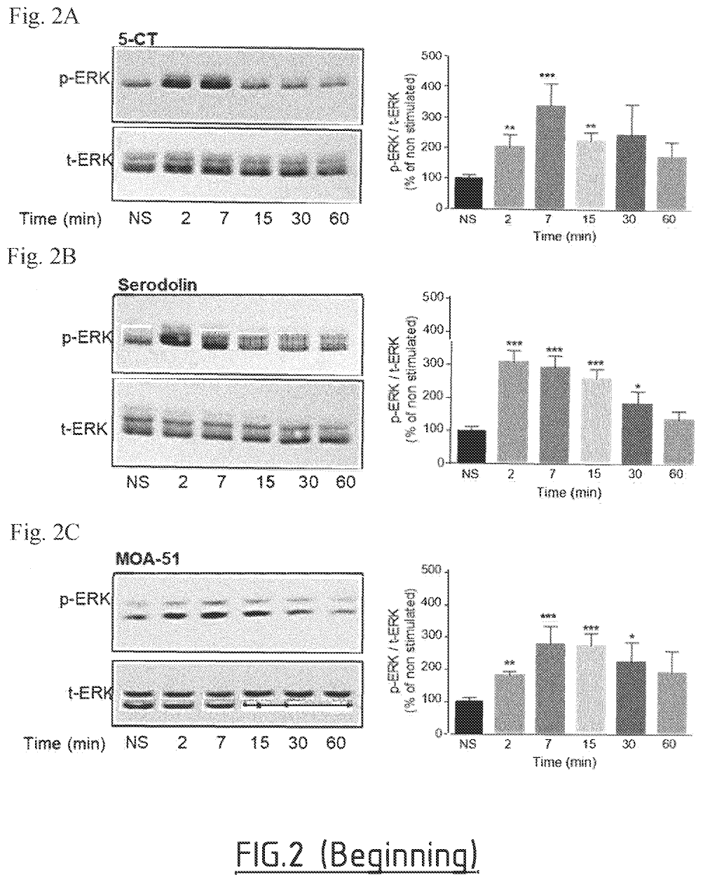

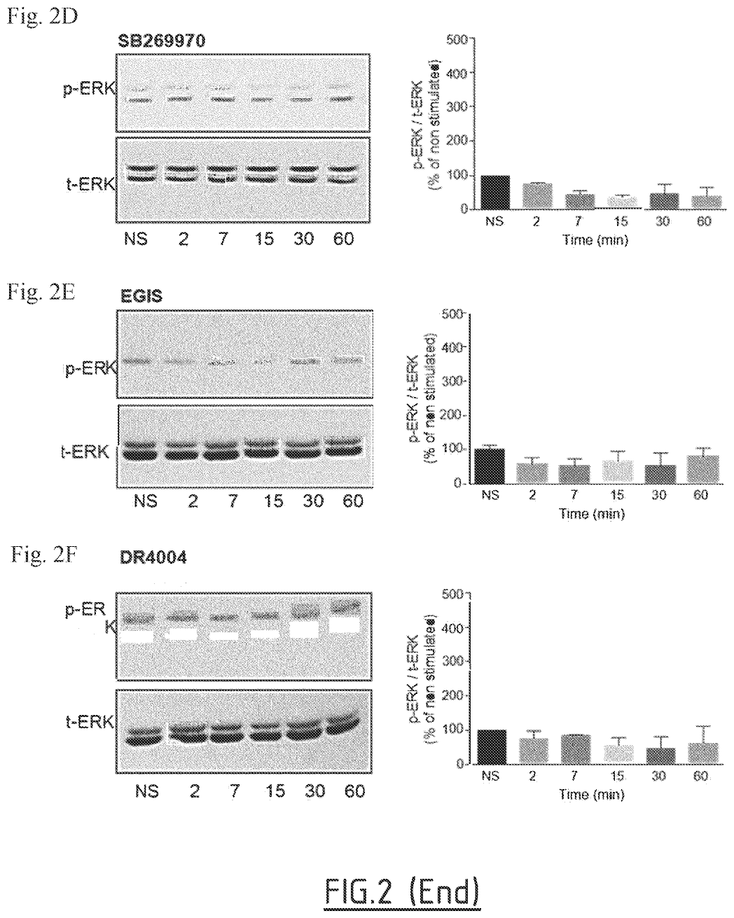

In mock-transfected HEK-293 cells, the inventors did not observe any modification of basal cAMP levels-induced by the ligands, consistent with the fact that HEK-293 cells do not expressed 5-HT₇R endogenously. Considering previous studies that demonstrated the activation of ERK pathway downstream of Gs coupling to 5-HT₇R, the effect of Serodolin and MOA-51 on ERK response was investigated. ERK phosphorylation was monitored by western blotting after treatment of cells with 10 μM of ligands at different times ranging from 2 to 60 minutes. As previously described (Lin, S. L., Johnson-Farley, N. N., Lubinsky, D. R., and Cowen, D. S. (2003) Coupling of neuronal 5-HT7 receptors to activation of extracellular-regulated kinase through a protein kinase A-independent pathway that can utilize Epac. J Neurochem 87, 1076-1085; Norum, J. H., Hart, K., and Levy, F. O. (2003) Ras-dependent ERK activation by the human G(s)-coupled serotonin receptors 5-HT4(b) and 5-HT7(a). J Biol Chem 278, 3098-3104), transient phosphorylation of ERK was observed upon 5-CT exposure (FIG. 2A). However, unexpectedly, Serodolin and MOA-51 were found to robustly induce ERK phosphorylation (409±34% and 278±55% of control at the maximal effect) (FIGS. 2B and 2C). Interestingly, when other known 5-HT₇R antagonist such as SB269970, EGIS or DR4004 were tested, none of them were able to induce ERK activation (FIG. 2 D, E, F), underlying the unique pharmacological profile of Serodolin and MOA-51. The kinetics of ERK phosphorylation elicited by 5-CT, Serodolin and MOA-51 were very similar: activation was fast and transient, and reached a pick between 2 and 7 minutes after drug exposure. However, ERK phosphorylation-induced by Serodolin and MOA-51 was more protracted compared to 5-CT, the reference agonist. This effect is not due to non-specific off-target effects, since neither Serodolin, nor MOA-51 were able to induce ERK phosphorylation when tested in HEK-293 mock cells.

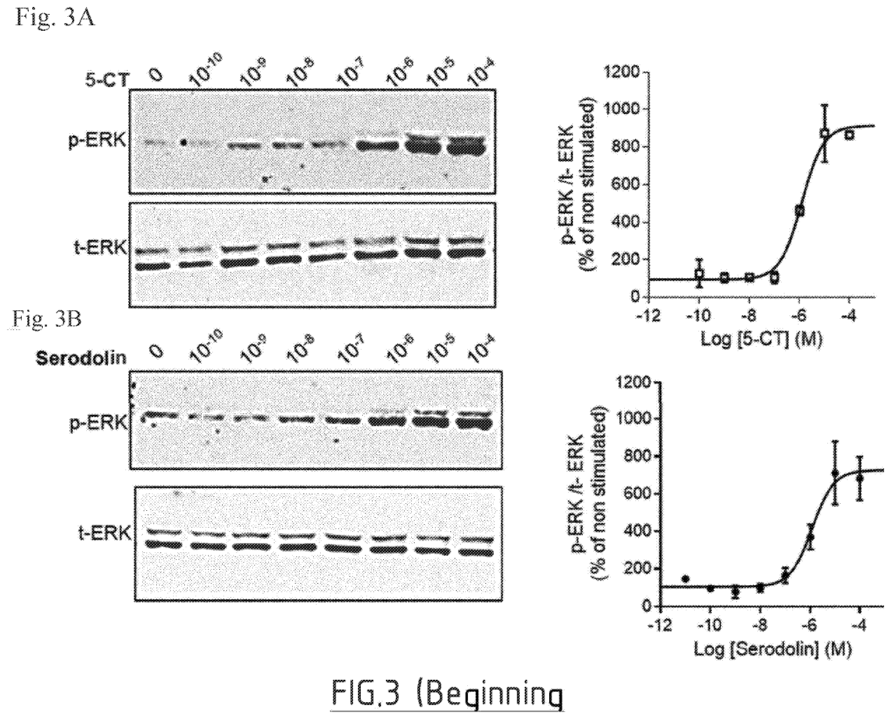

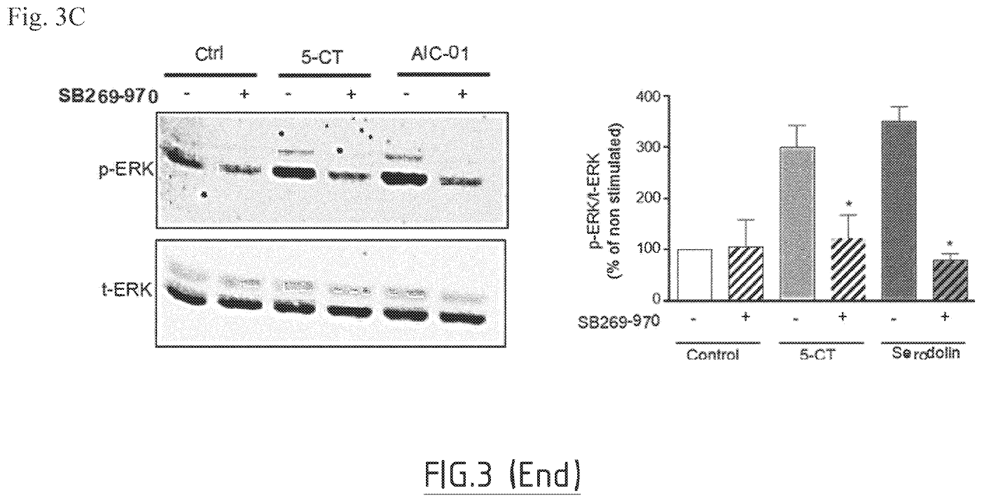

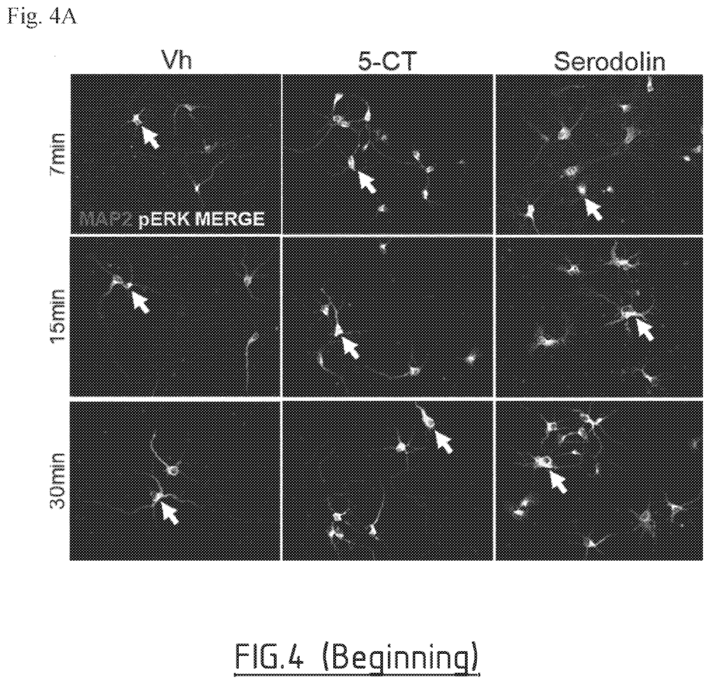

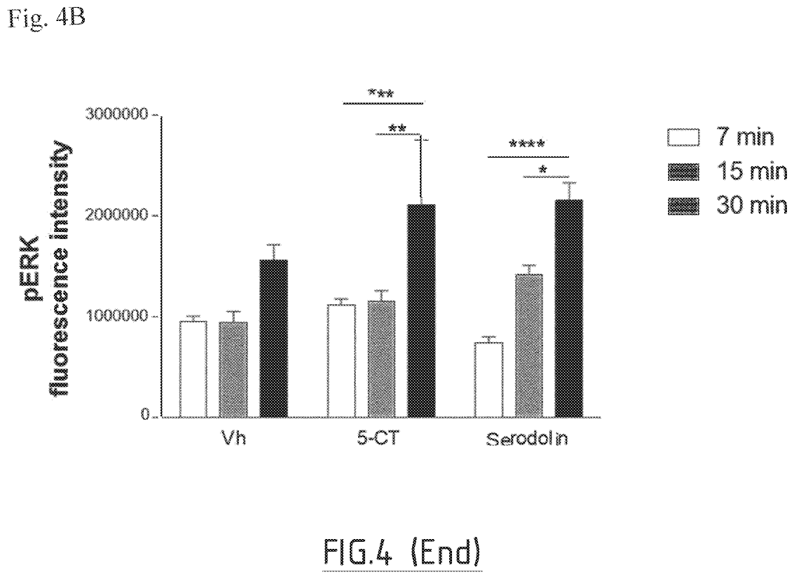

Similar to 5-CT, Serodolin induced a concentration-dependent increase of ERK phosphorylation in HEK-293 cells stably expressing 5-HT₇R (FIG. 3A-B). Moreover, this effect was fully blocked by co-incubation with SB-269970, a selective and highly potent 5-HT₇R antagonist (FIG. 3C). Importantly, the inventors demonstrated using immunocytochemistry that Serodolin-induced ERK phosphorylation also occurs in neuronal culture, endogenously expressing 5-HT₇R (FIG. 4A-B) and therefore is not limited to artificial cellular models overexpressing high levels of receptors. Collectively, these results revealed that Serodolin displays biased agonism at the 5-HT₇R: it behaves as antagonist/inverse agonist of AC pathway and as agonist on ERK phosphorylation.

### Serodolin-Induced ERK Through a G Protein-Independent Mechanism

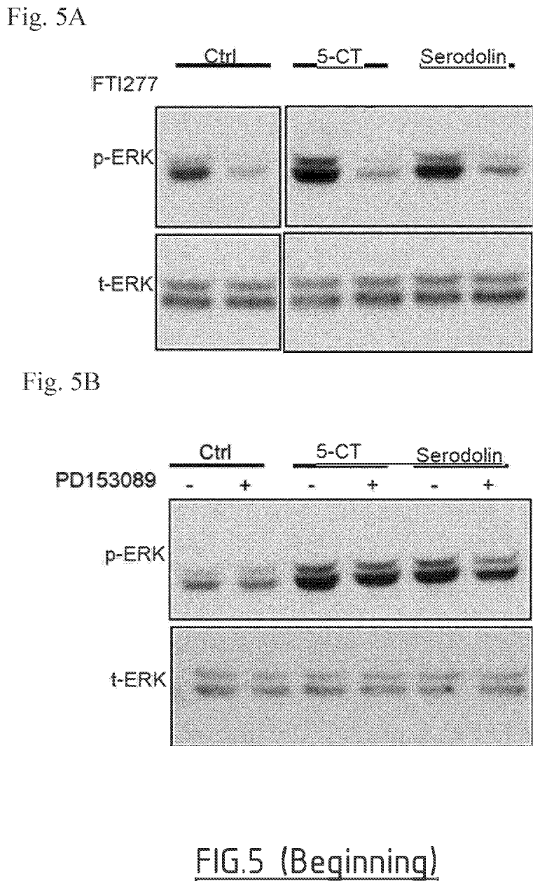

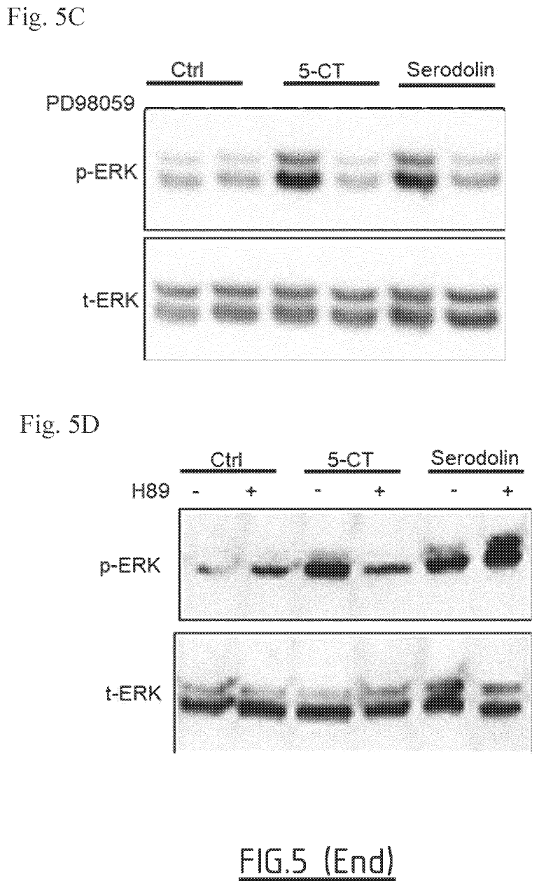

Because Gs/cAMP/PKA pathway was shown to contribute to ERK phosphorylation by conventional agonist 5-CT, we explored whether this was also the case for drug with biased efficacy like Serodolin. In agreement with previous observations, where MAPK activation by 5-CT have been shown to require Ras and MEK activation (Norum et al., 2003), the 5-CT response on ERK pathway could not be observed in cells pretreated with FT1277 or PD98059, selective Ras and MEK inhibitors respectively. Here, we demonstrated that Serodolin-induced ERK phosphorylation is also fully blocked by pretreatment with either of these kinase inhibitors, supporting a role of Ras and MEK in Serodolin downstream signaling pathway (FIGS. 5A and C). In the case of the stimulation of Gs-coupled receptor, the elevated levels of cAMP is known to induce activation of PKA which in turn induce ERK phosphorylation through a Ras-dependent mechanism. Several studies have shown that Gs-coupled receptors can activate MAPK cascade through EGFR transactivation (Kim, I. M., Tilley, D. G., Chen, J., Salazar, N. C., Whalen, E. J., Violin, J. D., and Rockman, H. A. (2008) Beta-blockers alprenolol and carvedilol stimulate beta-arrestin-mediated EGFR transactivation. Proc Natl Acad Sci USA 105, 14555-14560; Noma, T., Lemaire, A., Naga Prasad, S. V., Barki-Harrington, L., Tilley, D. G., Chen, J., Le Corvoisier, P., Violin, J. D., Wei, H., Lefkowitz, R. J., and Rockman, H. A. (2007) Beta-arrestin-mediated beta1-adrenergic receptor transactivation of the EGFR confers cardioprotection. J Clin Invest 117, 2445-2458). However, PD15035, a EGFR kinase inhibitor did not influence the 5-CT nor Serodolin-induced ERK phosphorylation (FIG. 5B). To examine the role of PKA, cells were preincubated with the PKA inhibitor H89 before the addition of 5-HT₇R ligands. As shown in FIG. 5D, the pretreatment of HEK293 cells with H89 partially decrease ERK phosphorylation induced by 5-CT whereas it had no effect on ERK phosphorylation induced by Serodolin.

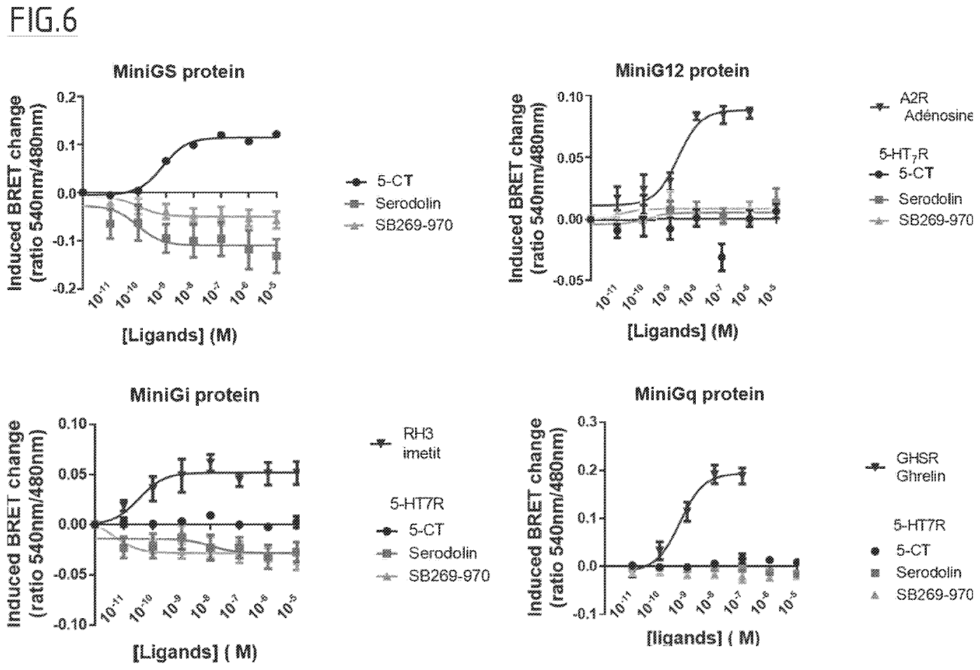

In order to explore other mechanisms involved in the biased effect of Serodolin, we decided to consider the ability of some GPCR ligands to ‘switch’ GPCR coupling from Gαs to Gαi, as observed for β adrenergic receptor (Daaka, Y., Luttrell, L. M., and Lefkowitz, R. J. (1997) Switching of the coupling of the beta2-adrenergic receptor to different G proteins by protein kinase A. Nature 390, 88-91). For that purpose, we evaluated the effect of Serodolin on the recruitment of different G proteins using variants of mini G (mG) proteins (mGs, mGsi, mGsq, and mG12) (Wan, Q., Okashah, N., Inoue, A., Nehme, R., Carpenter, B., Tate, C. G., and Lambert, N. A. (2018) Mini G protein probes for active G protein-coupled receptors (GPCRs) in live cells. J Biol Chem 293, 7466-7473), corresponding to the four families of Ga subunits and fused to a fluorescent protein, in BRET-based assay. We used the histamine H₃ receptor (H₃R), Adenosine 2 receptor (A₂R) or ghrelin receptor (GHSR) as positive controls for Gαi, Ga12 and Gαq recruitment. In order to assess the impact of kinetics, a critical aspect in the quantification of biased agonism (Klein Herenbrink, C., Sykes, D. A., Donthamsetti, P., Canals, M., Coudrat, T., Shonberg, J., Scammells, P. J., Capuano, B., Sexton, P. M., Charlton, S. J., Javitch, J. A., Christopoulos, A., and Lane, J. R. (2016) The role of kinetic context in apparent biased agonism at GPCRs. Nat Commun 7, 10842), cells were stimulated with increasing concentrations of compounds and BRET measurement was recorded in real-time over a 20 minutes. Then, the BRET signal obtained were plotted as concentration/response curve using values obtained at the end points (FIG. 6) The reference agonist 5-CT induced recruitment of Gs proteins with an EC₅₀ value (EC₅₀=1±1 nM), in agreement with that observed on AC pathway (FIG. 1B). In contrast, Serodolin as well as SB269970 behaved as inverse for Gαs recruitment. Whereas it was observed that stimulation of A₂R, H₃R and GHSR agonists can induce G proteins recruitment, neither classical (5-CT, SB 269970) nor biased 5-HT₇R ligands (Serodolin) were able to induce the Gαi, Ga12 or Gαq recruitment. In contrast, Serodolin behaves as inverse agonist for Gαs and Gαi coupling, suggesting that its effect on 5-HT₇R-induced ERK activation is mediated through a mechanism independent of G protein coupling. The inventors confirmed that 5-HT₇R-stimulated ERK1/2 activity did not depend on the Gq/IP3/Calcium pathway as no modification of intracellular calcium was observed after stimulation of 5-HT₇R with 5-CT or Serodolin in a calcium dependent bioluminescence sensor GFP-aequorin assay. In addition, we definitively excluded the role of Gi proteins in the activation of ERK cascade. Indeed, pretreatment of cells with the Gαi inhibitor pertussis toxin (PTX) had no effect on 5-HT₇R-mediated accumulation of p-ERK1/2. Altogether, our data indicate that Serodolin-mediated ERK phosphorylation in HEK-293 cells expressing 5-HT₇R does not require the generation of a classical second messenger dependent on Gs, Gi, G12 or Gq proteins.

Serodolin Triggers the Interaction of c-SRC-β-Arrestin Complex with a Proline-Rich Motif of 5-HT₇R Leading to ERK Phosphorylation

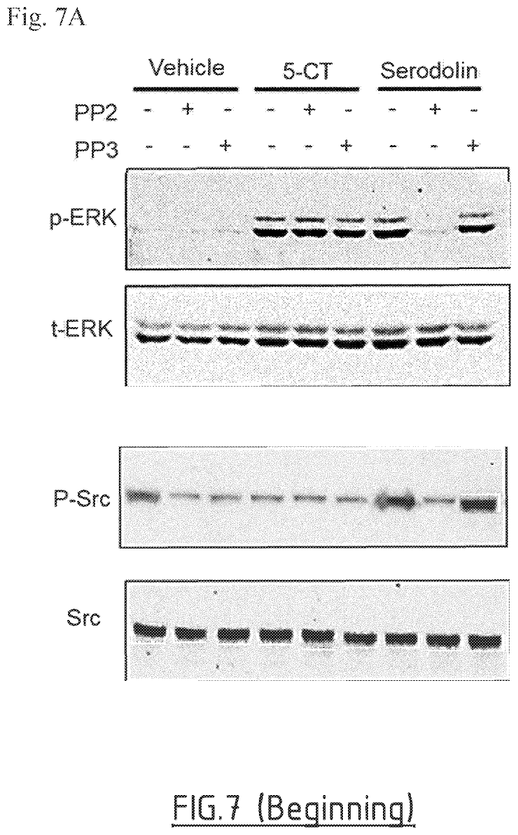

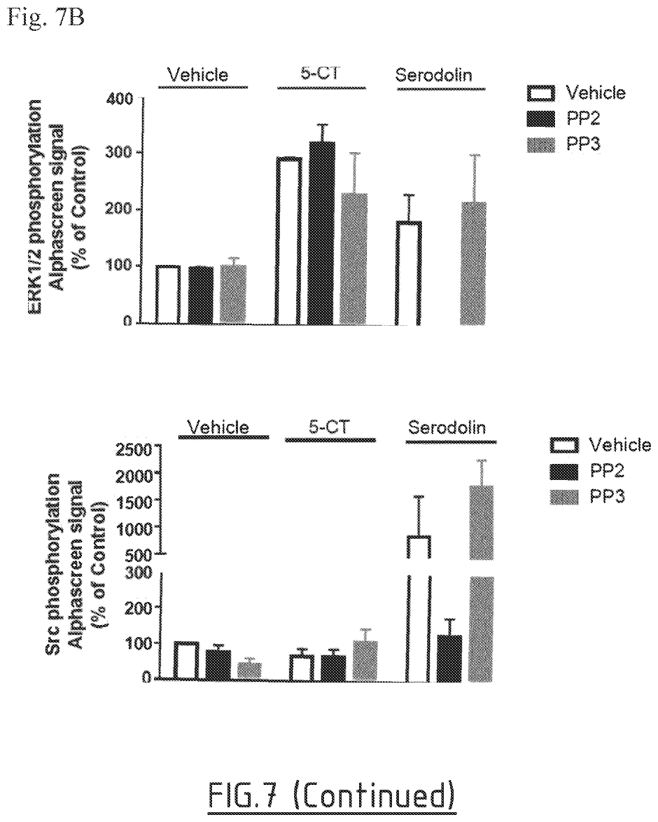

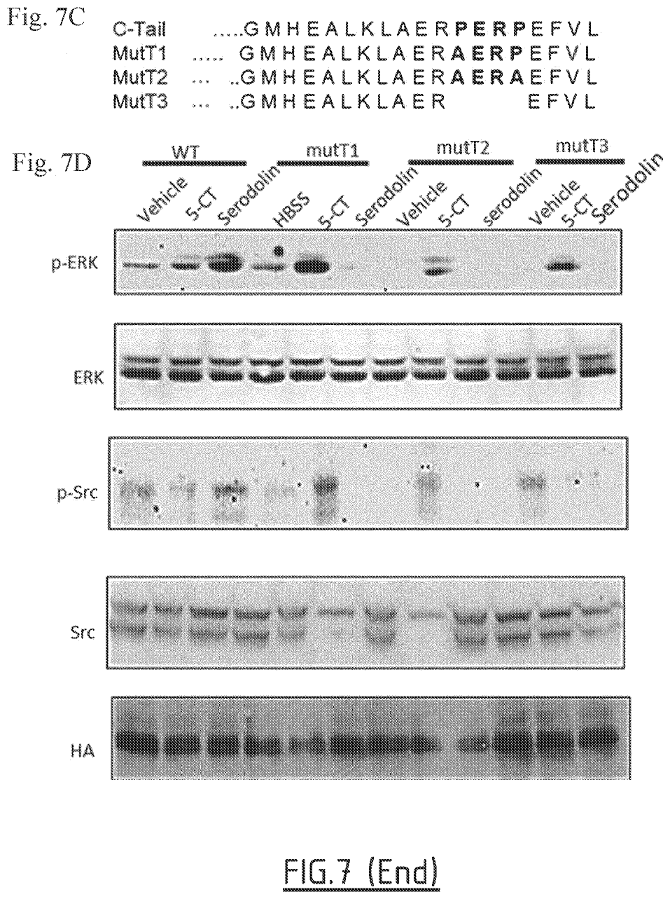

Interestingly, it was shown that some GPCRs can engage ERK1/2 activation through a scaffolding involving the receptor C-terminal part, c-SRC and β-arrestins (Barthet, G., Framery, B., Gaven, F., Pellissier, L., Reiter, E., Claeysen, S., Bockaert, J., and Dumuis, A. (2007) 5-hydroxytryptamine 4 receptor activation of the extracellular signal-regulated kinase pathway depends on Src activation but not on G protein or beta-arrestin signaling. Mol Biol Cell 18, 1979-1991; Perkovska, S., Mejean, C., Ayoub, M. A., Li, J., Hemery, F., Corbani, M., Laguette, N., Ventura, M. A., Orcel, H., Durroux, T., Mouillac, B., and Mendre, C. (2018) V1b vasopressin receptor trafficking and signaling: Role of arrestins, G proteins and Src kinase. Traffic 19, 58-82; Rey, A., Manen, D., Rizzoli, R., Caverzasio, J., and Ferrari, S. L. (2006) Proline-rich motifs in the parathyroid hormone (PTH)/PTH-related protein receptor C terminus mediate scaffolding of c-Src with beta-arrestin2 for ERK1/2 activation. J Biol Chem 281, 38181-38188). In many cases, s-arrestins function as a scaffold for c-SRC mediated activation of MAPKs (DeFea, K. A., Vaughn, Z. D., O'Bryan, E. M., Nishijima, D., Dery, O., and Bunnett, N. W. (2000) The proliferative and antiapoptotic effects of substance P are facilitated by formation of a beta-arrestin-dependent scaffolding complex. Proc Natl Acad Sci USA 97, 11086-11091; Luttrell, L. M., and Lefkowitz, R. J. (2002) The role of beta-arrestins in the termination and transduction of G-protein-coupled receptor signals. Journal of cell science 115, 455-465; and Yang, F., Xiao, P., Qu, C. X., Liu, Q., Wang, L. Y., Liu, Z. X., He, Q. T., Liu, C., Xu, J. Y., Li, R. R., Li, M. J., Li, Q., Guo, X. Z., Yang, Z. Y., He, D. F., Yi, F., Ruan, K., Shen, Y. M., Yu, X., Sun, J. P., and Wang, J. (2018) Allosteric mechanisms underlie GPCR signaling to SH3-domain proteins through arrestin. Nat Chem Biol 14, 876-886). Alternatively, c-SRC may be directly activated by binding to GPCR in the absence of β-arrestin (Cao, W., Luttrell, L. M., Medvedev, A. V., Pierce, K. L., Daniel, K. W., Dixon, T. M., Lefkowitz, R. J., and Collins, S. (2000) Direct binding of activated c-Src to the beta 3-adrenergic receptor is required for MAP kinase activation. J Biol Chem 275, 38131-38134). To determine whether one of these mechanisms was required for Serodolin-induced ERK phosphorylation, the inventors first evaluated the sensitivity of this activation to the c-SRC-specific tyrosine kinase inhibitor PP2. Importantly, pretreatment with PP2 inhibitor fully blocked the Serodolin-induced ERK phosphorylation whereas, in contrast, it had no effect on the 5-CT-stimulated ERK phosphorylation. PP3, a structural analogue of PP2 that does not inhibit c-SRC did not affect 5-HT₇R-dependent ERK and c-SRC phosphorylation. In addition, it was demonstrated that phosphorylation of c-SRC kinase at Tyr416 was induced only after activation of 5-HT₇R by Serodolin and could not be observed after 5-CT stimulation (FIG. 7A). The inventors confirmed the specific action of Serodolin on c-SRC activation by using the highly sensitive AlphaScreen Phospho assays. Using this approach, the Serodolin-induced ERK and c-SRC phosphorylation was fully blocked (by 99% and 100% respectively) in PP2 pretreatment cells, whereas PP3 was inactive (FIG. 7B). In contrast, the 5-CT induced-ERK phosphorylation was not sensitive to PP2 and did not induce c-SRC phosphorylation (FIGS. 7 A and B).

Previous studies have shown that proline-rich motifs (PXXP) in the third intracellular loop and the carboxyl terminus of GPCRs are involved in the recruitment of SH3-domain containing proteins (SH3-CPs), like c-SRC (Rey et al., 2006; Yang et al., 2014). Interestingly, the inventors identified such proline-rich motif in the sequence of the 5-HT₇R and aimed at dissecting its putative role in c-SRC and ERK activation. To generate proline deficient 5-HT₇R mutants, the proline-rich motif located in the end of the receptor C terminus to amino acid 425 was mutated one or two times to alanine (PXXP AXXP, mut1) and (PXXP AXXA, mut2) or fully deleted (mut3). The HA-tagged mutant receptors were well expressed at the plasma membrane and had cAMP response to 5-CT similar to WT. Interestingly, while all three mutants still responded to 5-CT by inducing ERK phosphorylation, they showed a complete loss of ERK and c-SRC phosphorylation upon Serodolin stimulation. Collectively, these results demonstrate the importance of the PXXP motif of 5-HT₇ receptor Ct in mediating Serodolin-induced signaling.

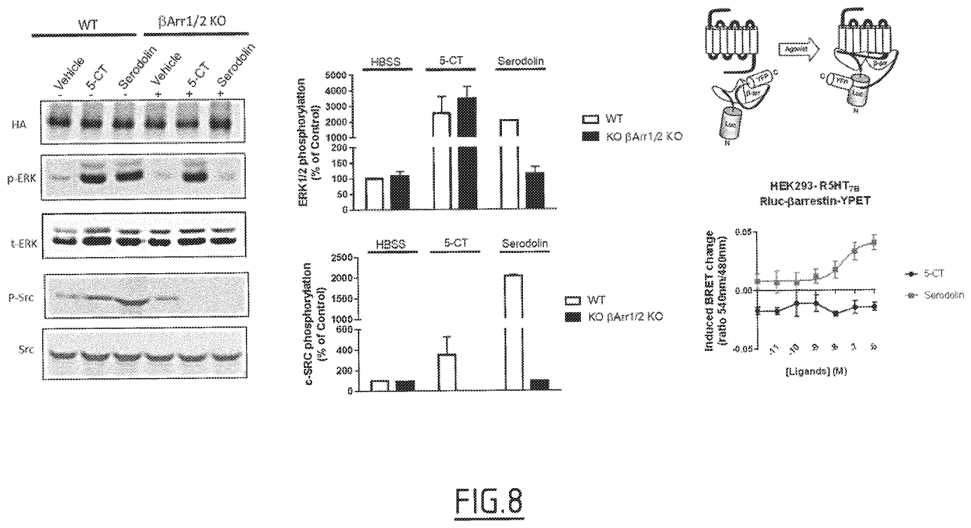

Previous studies have demonstrated that GPCRs can trigger non-G protein-mediated signaling events and in particular the activation of ERK1/2 by scaffolding complexes composed of c-SRC and β-arrestins. Considering the importance of c-SRC activation in Serodolin-mediated effect, we decided to determine whether β-arrestins are required for Serodolin-stimulated c-SRC-dependent ERK activation. Wild-type HEK-293 cells and β-arrestin deficient cells generated by using CRIPR-Cas9 gene editing were transfected with HA-5-HT₇R (FIG. 8A). Serodolin-induced ERK and c-SRC phosphorylation were abolished in β-arrestin KO cells, whereas absence of β-arrestins has no effect on 5-CT-induced responses analysed by Western blot (FIG. 8A) and Alphascreen assays (FIG. 8B). A siRNA knockdown approach was then employed to confirm the role of β-arrestins. The siRNA-mediated depletion of β-arrestins completely abrogated Serodolin-stimulated ERK and c-SRC phosphorylation, whereas it did not affect 5-CT-induced ERK phosphorylation.

The inventors then investigated whether the Serodolin-induced β-arrestin recruitement using BRET experiments. For that purpose the Renilla Luciferase sequence was fused to the C-terminal part of 5-HT₇R, and checked that the membrane expression and Gs/cAMP coupling of the fusion receptor was not modified (data not shown). After transfection of 5-HT₇R-RLuc and arrestin2-YFP used as a BRET pair, the inventors evaluated the ability of 5-HT₇R ligands to recruit β-arrestin. However, none of the ligands tested were able to induce an increase of BRET signal. We speculated that this lack of effect could be due to the addition of the RLuc at the end of the receptor Ct, which may affect C-terminal conformation of the receptor and/or prevent the accessory proteins to PXXP motifs. Therefore, to evaluate β-arrestin function with wild type 5-HT₇R, we used an intramolecular fluorescent BRET biosensor RLuc-arrestin2-YPET (Charest, P. G., Terrillon, S., and Bouvier, M. (2005) Monitoring agonist-promoted conformational changes of beta-arrestin in living cells by intramolecular BRET. EMBO Rep 6, 334-340). Interestingly, in contrast to 5-CT or SB269970, Serodolin was able to induce a concentration-dependent increase of BRET signal. BRET signal may reflect changes of the conformational states of β-arrestin induced by 5-HT₇R activation or may be due to steric interference of the donor and acceptor by recruitment of other binding partners as well as changes in the subcellular environment of the biosensor. However, corroborating results obtained with silencing β-arrestins, BRET analysis demonstrate a critical role of β-arrestin in mediating Serodilin signaling.

### Serodolin Reduces Nociception Through 5-HT₇R Biased Signaling

Several studies have suggested that systemic administration of 5-HT₇R agonists resulted in anti-allodynic and anti-hyperalgesic effects in pain conditions involving central sensitization. In line of these data, spinal blockade of 5-HT₇R has been reported to inhibit the anti-nociceptive effect of opioids supporting the idea that 5-HT₇R play an important role in physiological mechanisms controlling nociception and pain. Therefore the inventors aimed at evaluating the anti-nociceptive profile of Serodolin in different types of pain in mice (Viguier, F., Michot, B., Hamon, M., and Bourgoin, S. (2013) Multiple roles of serotonin in pain control mechanisms-implications of 5-HT(7) and other 5-HT receptor types. Eur J Pharmacol 716, 8-16).

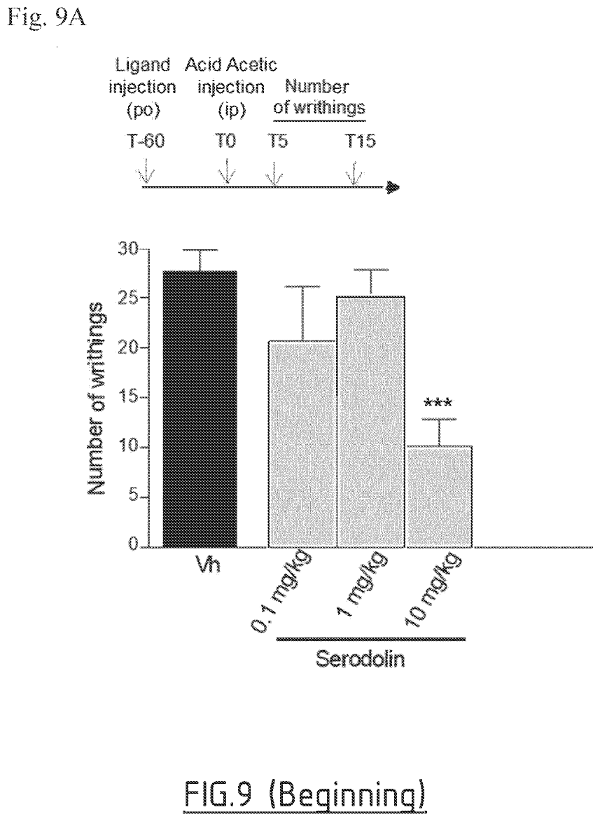

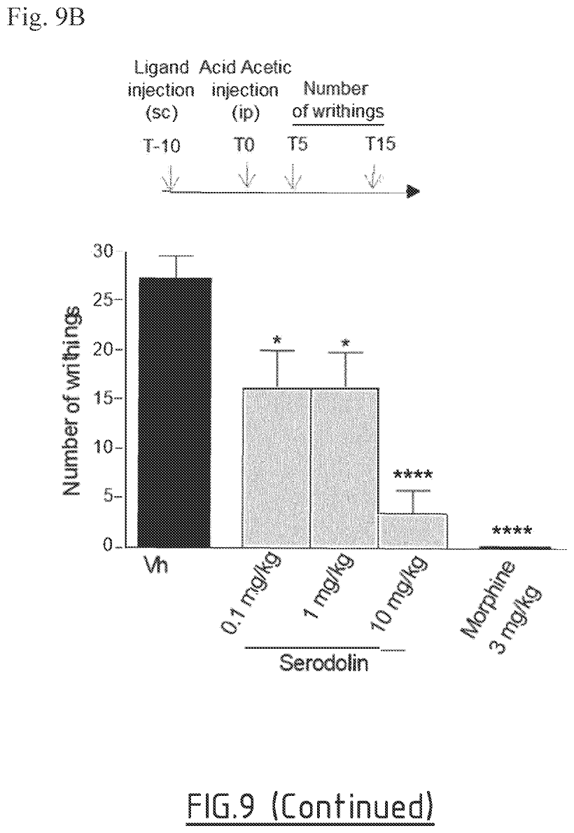

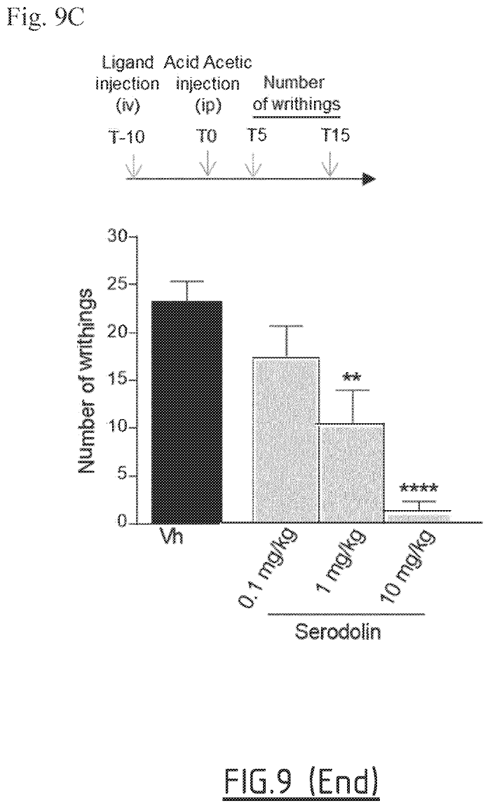

Analgesic activity was first evaluated using the acetic acid abdominal constriction test (writhing test), a chemical model of visceral pain. We evaluated the dose-response effect of Serodolin following single oral, intravenous and subcutaneous administration of the compound one hour before injection of acetic acid. Pretreatment of the mice with Serodolin produced a dose-dependent decrease of the acetic acid-induced writhing with a significant effect, even at the lower dosage tested, ie 0.1 mg/kg s.c. Interestingly, we demonstrated that at the highest dosage Serodolin was able to inhibit by up to 87% the writhing assay response as compared to the full inhibition produced by morphine (3 mg/kg, s.c.), supporting the therapeutic interest of Serodolin (FIG. 9).

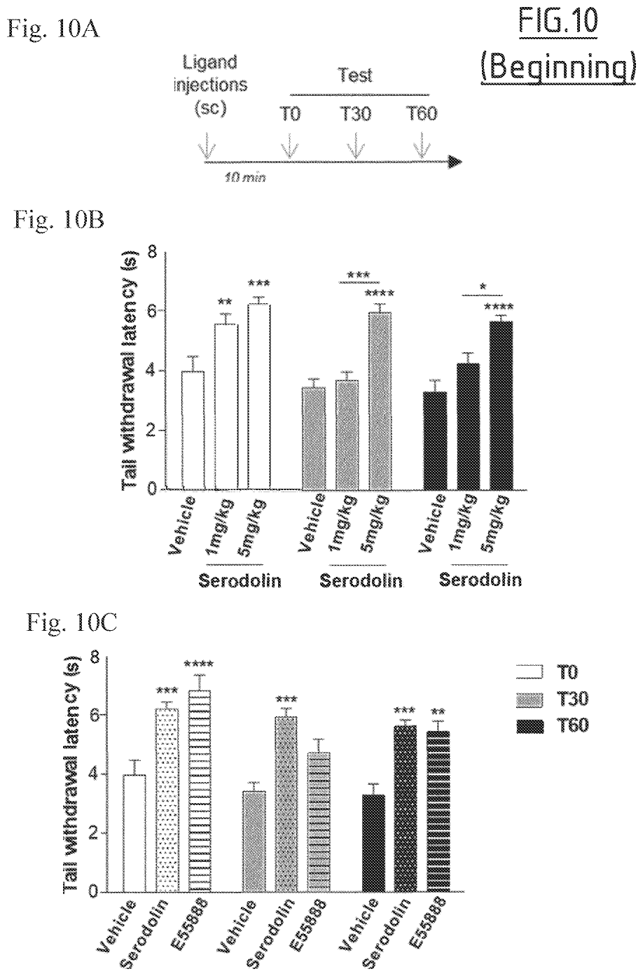

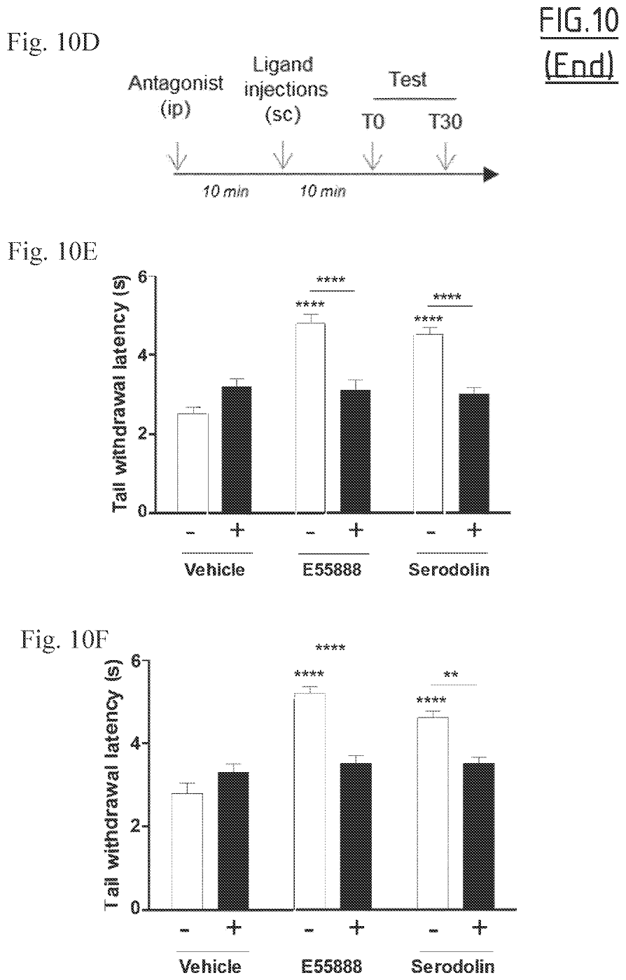

Thermal pain models in male mice were then used to investigate the mechanisms involved in the anti-nociceptive actions of Serodolin in comparison with E55888, a classical 5-HT₇R agonist with excellent selectivity profile. The mean pooled baseline tail-immersion latency of all the treatment groups was 3.6±0.4 secondes. Systemic administration of Serodolin (1 and 5 mg/kg, s.c.) produced a significant dose-dependent increase in the tail-immersion latencies (FIG. 10B). The effect of Serodolin begun within 10 min post administration and lasted beyond 70 minutes when given in dose of 5 mg/kg. In comparison with the effect of E-55888, Serodolin displayed almost identical behaviour in on the hot-water-immersion tail-flick latencies (FIG. 10C). To validate that the specificity of action of both compounds on 5-HT₇R, we evaluated whether SB269970, the selective and potent 5-HT₇ antagonist could reverse their anti-nociceptive properties (FIG. 10D). While SB-269970 administrated alone did not exert any significant effect on acute thermal nociception, it reversed the analgesia induced by either E-55888 or Serodolin at both time tested (FIGS. 10 E and F). To reinforce the role played by 5-HT₇R in Serodolin mediating analgesia, we tested its effect in homozygous mice carrying a deletion in the 5-HT₇R gene (5-HT₇R KO mice). The subcutaneous administration of E-55888 and Serodolin did not exert any anti-nociceptive effect in 5-HT₇R KO mice. These finding clearly establish that systemic administration Serodolin, a p arrestin biased 5-HT₇R ligand can generate behavioural anti-nociception through biased 5-HT₇R activation.

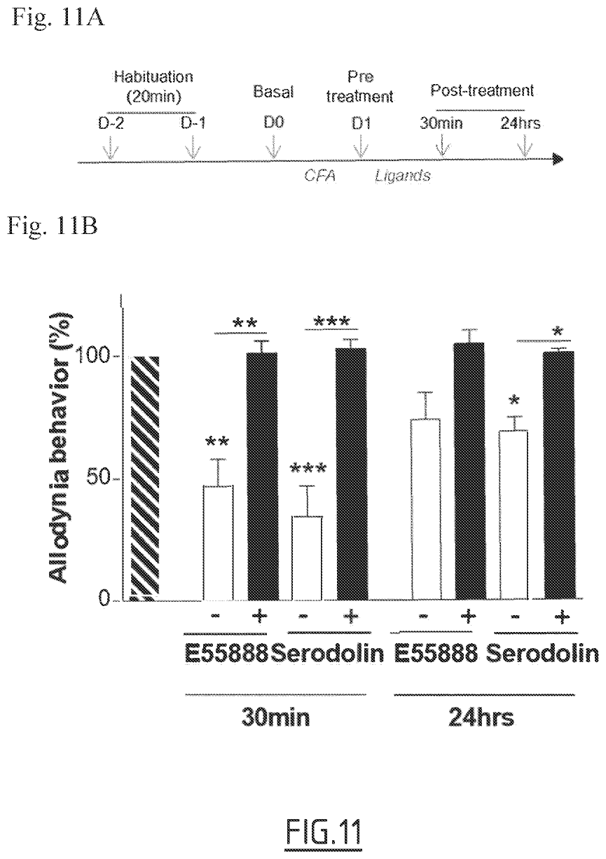

The inventors further explored the anti-nociceptive activity of Serodolin in vivo by evaluating its effect in the control of hypersensitivity following CFA sensitization. Mice injected with CFA into the midplantar surface of the right hind paw (ipsilateral paw) developed mechanical hypersensitivity, evidenced by a reduction (>50%) of the mechanical threshold triggering withdrawal of the ipsilateral paw in the Von Frey test 30 minutes after injection. No significant changes in the response to mechanical stimuli were observed in the contralateral paw (data not shown). The inventors wanted to evaluate the effect of Serodolin on mechanical hypersensitivity in comparison with E-55888 after CFA injection. As expected subcutaneous administration of E-55888 30 minutes after CFA injection reversed the CFA-induced mechanical hypersensitivity. Significantly decreased paw withdrawals thresholds (anti-allodynia) in mice treated with Serodolin were also observed 30 minutes after administration of 5 mg/kg, s.c. compared to vehicle-treated mice (FIG. 11). Interestingly, when tested 24 later administration of 5-HT₇R ligands, the anti-allodynic effect of Serodolin on CFA-induced hypersensitivity was still significant compared to E-55888. Importantly, pretreatment of animals with SB-268970 30 minutes before Serodolin or E55888 administration fully blocked their effect on allodynia.

It has been demonstrated here that Serodolin behaves as a 5-HT₇R biased ligand with dual efficacy. Indeed, a detailed pharmacological characterization revealed that Serodolin acts as a potent inverse agonist for Gs signaling while inducing an agonistic response for ERK pathway. The inventors reported here that the 5-CT-induced ERK activation requires Gs/cAMP/PKA/Ras signaling. In contrast, the Serodolin-induced ERK activation does not require G proteins activation. Rather, Serodolin reduces 5-HT₇R basal AC activity and inhibits its constitutive interaction with Gs protein, revealing a robust inverse agonist property. Of particular interest is the finding that other 5-HT₇ ligands defined as inverse agonists by their ability to decrease basal AC activity, were not able to induce ERK phosphorylation. Therefore, among 5-HT₇R ligands, the pharmacological profile of Serodolin is unique.

A convergent set of results is provided here indicating that Serodolin is able to reduce many aspects of pain-related behaviors such as mechanical allodynia or thermal hyperalgesia. Remarkably, in a peripheral inflammation murine model induced by hindpaw intraplantar injection of CFA, the anti-allodynic effects of Serodolin were as efficient and long lasting as E55888, a reference agonist compound of 5-HT₇R. The inventors demonstrated the specific action of Serodolin at 5-HT₇R as its effect were fully blocked by SB269970, an antagonist of 5-HT₇R. These data demonstrated for the first time the interests of a biased 5-HT₇ ligand in the inhibition of the transmission of pain signal.

### Use of 5-Ht7 Biased Ligand in Multiple Sclerosis

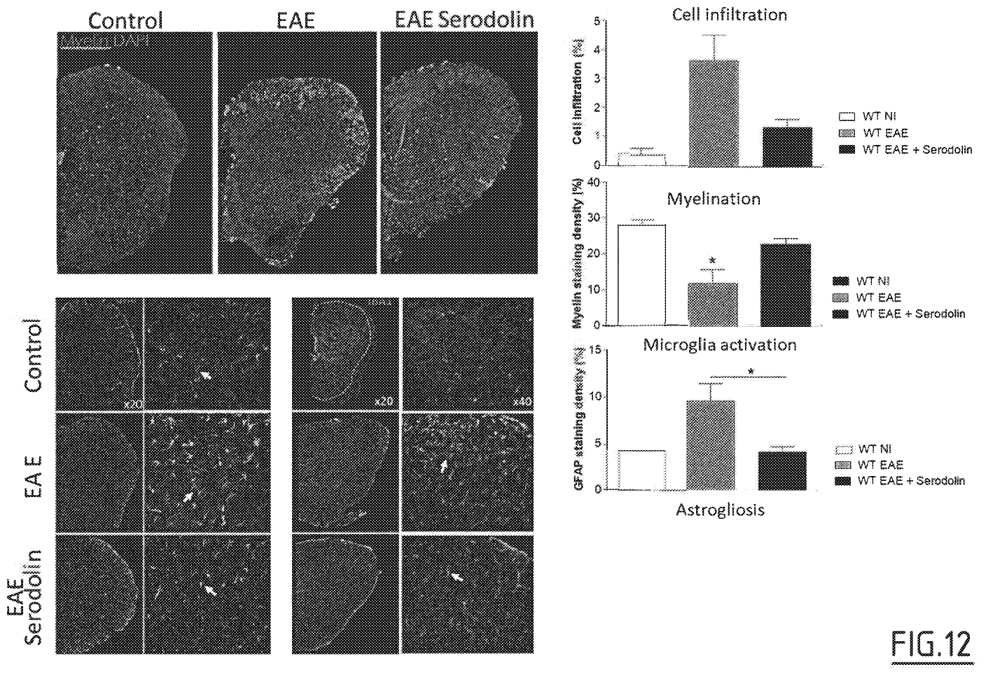

The inventors considered that Serodolin could have some benefit effects on some chronic inflammatory processes, like those observed in MS. Myelin oligodendrocyte glycoprotein (MOG)-induced murine experimental autoimmune encephalomyelitis (EAE) is a widely accepted model for studying the clinical and pathological features of multiple sclerosis. After MOG induction of EAE (9 animals/group), the effect of AIC-01 injection was tested for 9 days (1 mg/kg, ip from day 8 to day 18 after MOG induction). Each animal was assessed by a behavioural test based on motor functions, and an EAE score was obtained. After a 9 day-treatment started just before onset of clinical disabilities, the Serodolin treated group tended to show a delay in the onset of symptoms with a perceptible downtrend of the scores compared to Vehicle-treated EAE animals. Histopathological characteristics performed after MOG-induction allowed to decipher Serodolin effect at molecular and cellular levels. Spinal cord sections from control (NI: non MOG-induced) and treated EAE animals with or without Serodolin were labelled with both fluoromyelin and DAPI to evaluate myelin staining and cell infiltration, respectively. Activation of 5-HT₇R by Serodolin reduces cell infiltration and is associated with a higher myelin staining, which is increased by 65% over the level determined in the vehicle-treated animals (FIG. 12A) reflecting oligodendrocytes recruitment and/or protection through 5-HT₇ signaling. DAPI stained sections revealed higher cell infiltrates in the vehicle group compared with animals treated with Serodolin. Astrocytes and microglia staining were both significantly decreased in Serodolin-treated animals compared with the vehicle-treated mice (FIG. 12B). Altogether, these results underlie the potential interest of targeting the 5-HT₇R as a new strategy in MS therapy.

### Use of 5-Ht7 Biased Ligand in Thermoregulation

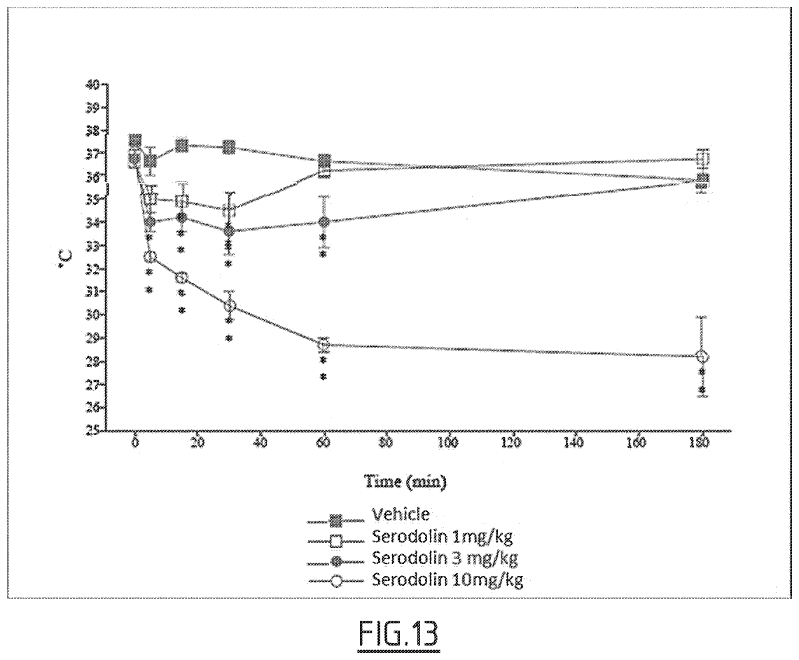

The 5-HT₇R is highly expressed in the preoptic area and anterior hypothalamus hypothalamus (Oliver, K. R., Kinsey, A. M., Wainwright, A., McAllister, G., Sirinathsinghji, D., (1999). Localisation of 5-HT7 and 5-HT5A receptor immunoreactivity in the rat brain. Society for Neuroscience Abstracts 25, 1207A), which are brain regions that play a key role in integrating central and peripheral mechanisms of thermoregulation The first indication that the 5-HT7 receptor is important in 5-HT-induced hypothermia was provided by the fact that the effect of 5-CT on body temperature was blocked by the selective antagonists SB269970, whereas SB266970 has no effect on rectal temperature when given alone (Hagan, J. J., Price, G. W., Jeffrey, P., Deeks, N.J., Stean, T., Piper, D., Smith, M. I., Upton, N., Medhurst, A. D., Middlemiss, D. N., Riley, G. J., Lovell, P. J., Bromidge, S. M., Thomas, D. R., (2000). Characterization of SB-269970-A, a selective 5-HT7 receptor antagonist. British Journal of Pharmacology 130, 539-548. and SB656104 [39] in guinea pigs. Furthermore, 5-HT and 5-CT failed to induce hypothermia in 5-HT7 receptor knockout mice (Guscott, M. R. et al. (2003). The hypothermic effect of 5-CT in mice is mediated through the 5-HT7 receptor. Neuropharmacology 44, 1031-1037). Considering the biased property of Serodolin, we investigated whether it behaves as 5-CT or SB 269970 on body temperature. The time course of the effect of Serodolin on the body temperature has been evaluated in the mouse following intravenous administration. When Serodolin was administered at the dose of 10 mg/kg, a marked (Emax: −7.9° C. at 60 min) and long lasting decrease in body temperature was observed, statistically significant from 5 min post dosing up to the end of observations (180 min) (FIG. 13). At the intermediate dose of 3 mg/kg, Serodolin induced a clear-cut decrease in body temperature (Emax: −3.6° C. at 30 min), statistically significant up to 60 min post dosing. Serodolin at the lowest dose of 1 mg/kg induced a transient decrease in body temperature (Emax: −2.9° C. at 30 min), statistically significant at 30 and 60 min post dosing. In conclusion, Serodolin induced a decrease in body temperature at and above the low dose of 1 mg/kg. This hypothermia was dose-dependent in intensity and duration. At the low dose, it was observed up to 60 min post dosing and at the top dose, a marked decrease was always observed at 180 min. Therefore, Serodolin produces a significant and dose-dependent reduction in body temperature as previously reported with the known 5-HT7 agonist, 5-CT.

### Preparation of Compounds According to the Invention

### General Information:

Commercially available reagents and solvents were used without further purification. Yields refer to isolated and purified products. Reaction were monitored by Thin Layer Chromatography (TLC) carried out on 60F-254 silica gel plates and visualized under UV light at 254 and 365 nm. Column chromatography was performed on a Buchi Pure C-810 Flash using pre-packed silica 40-63 μm columns. ¹H NMR spectra were recorded on a Brucker Avance DPX-250 (250 MHz) and a Brucker Avance 400 (400 MHz) spectrometer. ¹³C NMR spectra were recorded on a Brucker Avance 400 (101 MHz). ¹⁹F NMR spectra were recorded on a Brucker Avance 400 (356 MHz). Chemical shifts are reported in ppm and residual non deuterated solvents were used as references. Mutliplicities are designated by the following abbrevations: s=singlet, d=doublet, t=triplet, q=quadruplet, p=pentuplet, br s=broad singlet, m=multiplet. High-resolution mass analyses were performed on a Maxis Brucker spectrometer using electrospray ionisation (ESI). For chlorinated and brominated compounds, the given mass corresponds to the ³⁵Cl and ⁷⁹Br isotopes. Melting points were measured on a Thermo Scientific 9200 apparatus with capillary tubes. Purity of final compounds was measured by LC-MS using a C18 column (Waters Aquity BEH, 1.7 μm, 30×2.1 mm). Phase A: Water+0.1% formic acid; phase B: acetonitrile+0.1% formic acid. Flow rate: 0.5 mL/min. Elution gradient: t=0 s: 85% of phase A, 15% of phase B, t=90 s: 100% phase B, t=180 s: 100% of phase B.

General Procedure a for Nucleophilic Substitution with Piperazines

A solution of 1-(5-bromopentyl)-1H-benzo[d]imidaol-2(3H)-one (150 mg, 0.53 mmol, 1 eq.), piperazine (1.3 eq.) and DIPEA (0.23 mL, 2.5 eq.) in acetonitrile (2.3 mL) was refluxed for 2 hours. The solvent was removed under reduced pressure. The residue was diluted in water and extracted with DCM (3×10 mL). The organic layer was dried over anhydrous MgSO₄, concentrated under reduced pressure and purified by column chromatography using a gradient of DCM to DCM/MeOH 10% to Afford the Corresponding Product.

General Procedure B for Buchwald Coupling with Piperazine Analogs

In a sealed tube, tert-butyl-1,4-diazepane-1-carboxylate (100 mg, 0.5 mmol, 1 eq.), 1-bromo-4-fluorobenzene (175 mg, 2 eq.), Pd₂dba₃ (4.6 mg, 1 mol %), RuPhos (4.7 mg, 2 mol %) and t-BuONa (144 mg, 3 eq.) were suspended in dioxane (1.5 mL). The solution was sonicated for 2 min and then heated for 20 min at 100° C. The reaction mixture was filtered on Celite, washed with DCM. The solvents were removed under reduced pressure and the crude was directly purified by flash chromatography (PE to PE/EA 8:2) to afford the corresponding product.

### General Procedure C for Boc Deprotection of Piperazine Analogs

To a solution of N-Boc piperazine analog (0.44 mmol, 1 eq.) in DCM (3 mL) was added TFA (3 mL). The mixture was stirred at rt for 20 min, then quenched with a NaHCO₃ saturated solution. The aqueous phase was extracted with DCM. The organic layer was dried over anhydrous MgSO₄, concentrated under reduced pressure to afford the desired compound without further purification.

### 1-(5-(4-(2-hydroxyphenyl)piperazin-1-yl)pentyl)-1H-benzo[d]imidazol-2(3H)-one

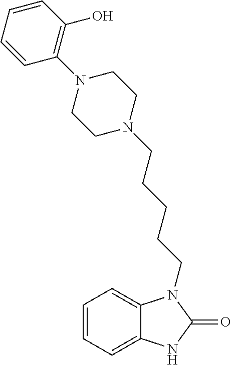 [SMILES: O=c1[nH]c2ccccc2n1CCCCCN1CCN(c2ccccc2O)CC1]

The reaction was performed according to general procedure A. Beige solid (194 mg, 96%).

¹H NMR (400 MHz, CDCl₃) δ 9.46 (s, 1H), 7.16 (dd, J=7.9, 1.5 Hz, 1H), 7.14-7.02 (m, 4H), 7.03-6.97 (m, 1H), 6.94 (dd, J=8.1, 1.5 Hz, 1H), 6.85 (td, J=7.6, 1.5 Hz, 1H), 3.91 (t, J=7.2 Hz, 2H), 2.90 (t, J=4.8 Hz, 4H), 2.61 (br s, 4H), 2.42 (t, J=7.5 Hz, 2H), 1.82 (p, J=7.5 Hz, 2H), 1.60 (p, J=7.5 Hz, 2H), 1.45 (q, J=8.0 Hz, 2H).

¹³C NMR (101 MHz, CDCl₃) δ 155.52, 151.65, 139.11, 130.54, 128.01, 126.59, 121.64, 121.55, 121.45, 120.18, 114.14, 109.65, 108.01, 58.54, 54.03, 52.60, 40.90, 28.38, 26.58, 24.90.

HRMS (ESI) m/z: calculated for C₂₂H₉N₄O₂ [M+H]⁺ 381.2285; found 381.2291

m.p.: 148° C.

### 1-(5-(4-(2-(trifluoromethyl)phenyl)piperazin-1-yl)pentyl)-1H-benzo[d]imidazol-2(3H)-one

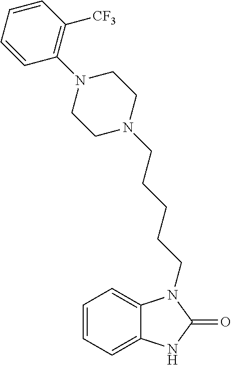 [SMILES: O=c1[nH]c2ccccc2n1CCCCCN1CCN(c2ccccc2C(F)(F)F)CC1]

The reaction was performed according to general procedure A. Beige solid (193 mg, 84%).

¹H NMR (400 MHz, CDCl₃) δ 9.41 (s, 1H), 7.61 (dd, J=7.9, 1.6 Hz, 1H), 7.50 (td, J=7.7, 1.6 Hz, 1H), 7.38 (d, J=8.0 Hz, 1H), 7.21 (t, J=7.6 Hz, 1H), 7.14-7.03 (m, 3H), 7.03-6.97 (m, 1H), 3.90 (t, J=7.2 Hz, 2H), 2.99 (t, J=4.7 Hz, 4H), 2.74-2.55 (br s, 4H), 2.45 (t, J=7.6 Hz, 2H), 1.82 (p, J=7.6 Hz, 2H), 1.63 (p, J=7.6 Hz, 2H), 1.44 (qd, J=9.8, 9.1, 6.3 Hz, 2H).

¹³C NMR (101 MHz, CDCl₃) δ 155.48, 152.57, 132.89, 130.54, 127.99, 127.33, 125.54, 124.96, 124.22, 121.55, 121.46, 109.64, 108.02, 58.54, 53.65, 53.24, 40.88, 28.37, 26.36, 24.91.

¹⁹F NMR (356 MHz, CDCl₃) δ −60.38.

HRMS (ESI) m/z: calculated for C₂₃H₃F₃N₄O [M+H]⁺ 433.2210; found 433.2211

m.p.: 102° C.

### 1-(5-(4-(2-fluorophenyl)piperazin-1-yl)pentyl)-1H-benzo[d]imidazol-2(3H)-one

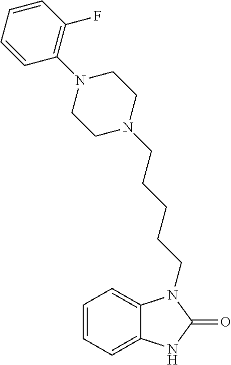 [SMILES: O=c1[nH]c2ccccc2n1CCCCCN1CCN(c2ccccc2F)CC1]

The reaction was performed according to general procedure A. Beige solid (155 mg, 76%).

¹H NMR (400 MHz, CDCl₃) δ 9.39 (s, 1H), 7.14-6.96 (m, 6H), 6.99-6.89 (m, 2H), 3.90 (t, J=7.3 Hz, 2H), 3.12 (t, J=4.8 Hz, 4H), 2.65 (t, J=4.8 Hz, 4H), 2.43 (t, J=7.6 Hz, 2H), 1.82 (p, J=7.3 Hz, 2H), 1.62 (p, J=7.6 Hz, 2H), 1.52-1.37 (m, 2H).

¹³C NMR (101 MHz, CDCl₃) δ 157.10, 155.47, 154.65, 140.20 (d, J=8.2 Hz), 129.27 (d, J=258.3 Hz), 124.59 (d, J=3.5 Hz), 122.59 (d, J=7.4 Hz), 121.50 (d, J=9.1 Hz), 119.09 (d, J=3.0 Hz), 116.22 (d, J=20.8 Hz), 109.63, 108.01, 58.54, 53.40, 40.89, 28.38, 26.44, 24.90.

¹⁹F NMR (356 MHz, CDCl₃) δ −122.77

HRMS (ESI) m/z: calculated for C₂₂H₈FN₄O [M+H]⁺ 383.2242; found 383.2248

m.p.: 138° C.

### 1-(5-(4-(p-tolyl)piperazin-1-yl)pentyl)-1H-benzo[d]imidazol-2(3H)-one

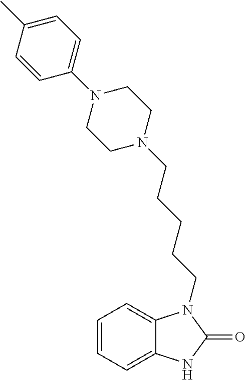 [SMILES: Cc1ccc(N2CCN(CCCCCn3c(=O)[nH]c4ccccc43)CC2)cc1]

The reaction was performed according to general procedure A. Beige solid (132 mg, 66%).

¹H NMR (400 MHz, CDCl₃) δ 9.43 (s, 1H), 7.13-7.01 (m, 5H), 7.00 (d, J=7.2 Hz, 1H), 6.83 (d, J=8.1 Hz, 2H), 3.90 (t, J=7.2 Hz, 2H), 3.14 (t, J=4.9 Hz, 4H), 2.59 (t, J=4.9 Hz, 4H), 2.43-2.35 (m, 2H), 2.26 (s, 3H), 1.82 (p, J=7.6 Hz, 2H), 1.60 (p, J=7.6 Hz, 2H), 1.43 (p, J=7.6 Hz, 2H).

¹³C NMR (101 MHz, CDCl₃) δ 155.49, 149.39, 130.55, 129.75, 129.32, 127.99, 121.52, 121.43, 116.53, 109.63, 108.00, 58.61, 53.44, 49.80, 40.92, 28.41, 26.61, 24.96, 20.55.

HRMS (ESI) m/z: calculated for C₂₃H₃₁N₄O [M+H]⁺ 379.2492; found 379.2496

m.p.: 163° C.

### 1-(5-(4-(4-(trifluoromethyl)phenyl)piperazin-1-yl)pentyl)-1H-benzo[d]imidazol-2(3H)-one

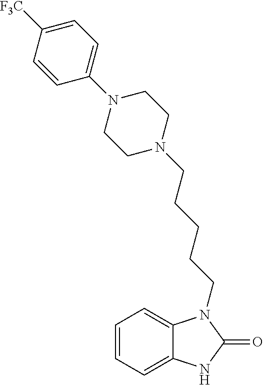 [SMILES: O=c1[nH]c2ccccc2n1CCCCCN1CCN(c2ccc(C(F)(F)F)cc2)CC1]

The reaction was performed according to general procedure A. Beige solid (171 mg, 75%).

¹H NMR (400 MHz, CDCl₃) δ 9.83 (s, 1H), 7.46 (d, J=8.6 Hz, 1H), 7.13-7.05 (m, 3H), 7.00 (dd, J=6.7, 1.9 Hz, 1H), 6.89 (d, J=8.6 Hz, 2H), 3.91 (t, J=7.4 Hz, 1H), 3.26 (t, J=5.1 Hz, 4H), 2.57 (t, J=5.1 Hz, 4H), 2.38 (t, J=7.4 Hz, 2H), 1.82 (p, J=7.4 Hz, 2H), 1.60 (p, J=7.4 Hz, 2H), 1.49-1.37 (m, 2H).

¹³C NMR (101 MHz, CDCl₃) δ 153.40, 130.48, 128.13, 126.51, 126.47, 121.57, 121.39, 114.57, 109.76, 108.00, 58.47, 53.04, 48.00, 40.85, 28.36, 26.49, 24.84.

¹⁹F NMR (356 MHz, CDCl₃) 5-61.35

HRMS (ESI) m/z: calculated for C₂₃H₈F₃N₄O [M+H]⁺ 433.2210; found 433.2217

m.p.: 144° C.

### 1-(5-(4-(3,4-dichlorophenyl)piperazin-1-yl)pentyl)-1H-benzo[d]imidazol-2(3H)-one

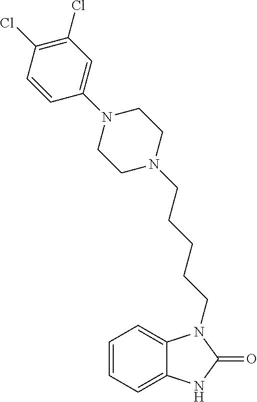 [SMILES: O=c1[nH]c2ccccc2n1CCCCCN1CCN(c2ccc(Cl)c(Cl)c2)CC1]

The reaction was performed according to general procedure A. Beige solid (178 mg, 77%).

¹H NMR (400 MHz, CDCl₃) δ 8.89 (s, 1H), 7.28-7.22 (m, 1H), 7.13-7.02 (m, 3H), 6.99 (d, J=7.1 Hz, 1H), 6.93 (d, J=2.8 Hz, 1H), 6.71 (dd, J=9.0, 2.8 Hz, 1H), 3.89 (t, J=7.4 Hz, 2H), 3.14 (t, J=5.0 Hz, 4H), 2.54 (t, J=5.0 Hz, 4H), 2.37 (t, J=7.4 Hz, 2H), 1.81 (p, J=7.4 Hz, 2H), 1.62-1.52 (m, 2H), 1.42 (p, J=7.9 Hz, 4H).

¹³C NMR (101 MHz, CDCl₃) δ 155.23, 150.86, 132.89, 130.60, 130.53, 127.85, 122.14, 121.55, 121.52, 117.27, 115.35, 109.52, 108.03, 58.45, 53.06, 48.76, 40.90, 28.35, 26.59, 24.87.

HRMS (ESI) m/z: calculated for C₂₂H₇Cl₂N₄O [M+H]⁺ 433.1556; found 433.1559

m.p.: 145° C.

### 1-(5-(4-(2,4-dichlorophenyl)piperazin-1-yl)pentyl)-1H-benzo[d]imidazol-2(3H)-one

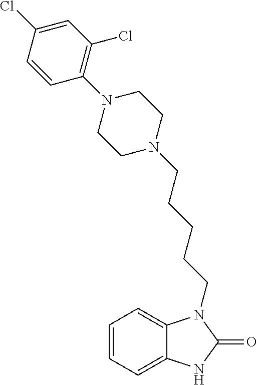 [SMILES: O=c1[nH]c2ccccc2n1CCCCCN1CCN(c2ccc(Cl)cc2Cl)CC1]

The reaction was performed according to general procedure A. Beige solid (200 mg, 87%).

¹H NMR (400 MHz, CDCl₃) δ 8.94 (s, 1H), 7.35 (d, J=2.4 Hz, 1H), 7.18 (dd, J=8.7, 2.4 Hz, 1H), 7.14-7.02 (m, 3H), 6.99 (dd, J=7.9, 1.8 Hz, 1H), 6.95 (d, J=8.7 Hz, 1H), 3.90 (t, J=7.5 Hz, 2H), 3.05 (s, 4H), 2.64 (s, 4H), 2.43 (t, J=7.5 Hz, 2H), 1.82 (p, J=7.5 Hz, 2H), 1.61 (p, J=7.5 Hz, 2H), 1.43 (p, J=7.5 Hz, 2H).

¹³C NMR (101 MHz, CDCl₃) δ 155.64, 148.13, 130.51, 130.42, 129.57, 128.34, 128.07, 127.75, 121.55, 121.41, 121.30, 109.71, 108.00, 58.47, 53.35, 51.14, 40.87, 28.37, 26.44, 24.87.

HRMS (ESI) m/z: calculated for C₂₂H₇N₄OCl₂ [M+H]⁺ 433.1556; found 433.1558

m.p.: 93° C.

### 1-(5-(4-(pyridin-2-yl)piperazin-1-yl)pentyl)-1H-benzo[d]imidazol-2(3H)-one

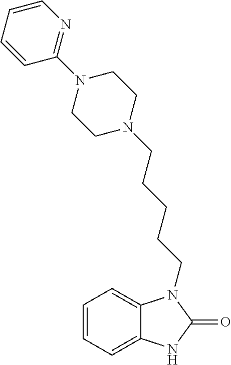 [SMILES: O=c1[nH]c2ccccc2n1CCCCCN1CCN(c2ccccn2)CC1]

The reaction was performed according to general procedure A. Beige solid (143 mg, 74%).

¹H NMR (400 MHz, CDCl₃) δ 8.50 (s, 1H), 8.18 (ddd, J=4.9, 2.0, 0.9 Hz, 1H), 7.46 (ddd, J=8.9, 7.1, 2.0 Hz, 1H), 7.12-7.04 (m, 3H), 7.01-6.96 (m, 1H), 6.66-6.57 (m, 2H), 3.89 (t, J=7.5 Hz, 2H), 3.54 (t, J=5.0 Hz, 4H), 2.54 (t, J=5.0 Hz, 4H), 2.38 (t, J=7.5 Hz, 2H), 1.80 (p, J=7.5 Hz, 2H), 1.67-1.54 (m, 2H), 1.44 (p, J=7.5 Hz, 2H).

¹³C NMR (101 MHz, CDCl₃) δ 159.59, 155.77, 148.07, 137.58, 130.47, 128.17, 121.53, 121.34, 113.47, 109.76, 107.96, 107.20, 58.56, 53.09, 45.13, 40.84, 28.35, 26.35, 24.86.

HRMS (ESI) m/z: calculated for C₂₁H₈N₅O [M+H]⁺ 365.2288; found 365.2288

m.p.: 155° C.

### 1-(5-(4-(pyridin-3-yl)piperazin-1-yl)pentyl)-1H-benzo[d]imidazol-2(3H)-one

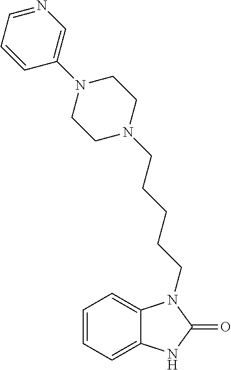 [SMILES: O=c1[nH]c2ccccc2n1CCCCCN1CCN(c2cccnc2)CC1]

The reaction was performed according to general procedure A. Beige solid (100 mg, 52%).

¹H NMR (400 MHz, CDCl₃) δ 10.52 (s, 1H), 8.29 (s, 1H), 8.08 (s, 1H), 7.16-7.08 (m, 3H), 7.07-7.03 (m, 1H), 7.00-6.96 (m, 1H), 3.89 (t, J=7.3 Hz, 2H), 3.20 (t, J=4.9 Hz, 4H), 2.58 (t, J=4.9 Hz, 4H), 2.38 (t, J=7.3 Hz, 2H), 1.81 (p, J=7.3 Hz, 2H), 1.58 (p, J=7.3 Hz, 2H), 1.42 (p, J=7.3 Hz, 2H).

¹³C NMR (101 MHz, CDCl₃) δ 155.86, 147.05, 140.61, 138.52, 130.44, 128.29, 123.58, 122.37, 121.46, 121.22, 109.73, 107.90, 58.40, 52.98, 48.38, 40.78, 28.32, 26.42, 24.79.

HRMS (ESI) m/z: calculated for C₂₁H₈N₅O [M+H]⁺ 366.2288; found 366.2288

m.p.: 125° C.

### 1-(5-(4-(pyridin-4-yl)piperazin-1-yl)pentyl)-1H-benzo[d]imidazol-2(3H)-one

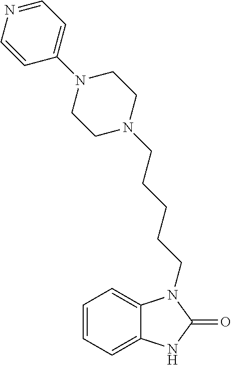 [SMILES: O=c1[nH]c2ccccc2n1CCCCCN1CCN(c2ccncc2)CC1]

The reaction was performed according to general procedure A. Orange oil (77 mg, 40%).

¹H NMR (400 MHz, CDCl₃) δ 10.20 (s, 1H), 8.24 (d, J=5.6 Hz, 2H), 7.12-7.02 (m, 3H), 7.04-6.93 (m, 1H), 6.62 (d, J=5.6 Hz, 2H), 3.90 (t, J=7.5 Hz, 2H), 3.31 (t, J=5.1 Hz, 4H), 2.51 (t, J=5.1 Hz, 4H), 2.36 (t, J=7.5 Hz, 2H), 1.81 (p, J=7.5 Hz, 2H), 1.57 (p, J=7.5 Hz, 2H), 1.42 (p, J=7.5 Hz, 2H).

¹³C NMR (101 MHz, CDCl₃) δ 155.73, 155.22, 149.43, 130.47, 128.23, 121.50, 121.30, 109.71, 108.32, 107.94, 58.35, 52.71, 45.97, 40.77, 28.29, 26.44, 24.75.

HRMS (ESI) m/z: calculated for C₂₁H₈N₅O [M+H]⁺ 366.2288; found 366.2291

### 1-(5-(4-(pyrimidin-4-yl)piperazin-1-yl)pentyl)-1H-benzo[d]imidazol-2(3H)-one

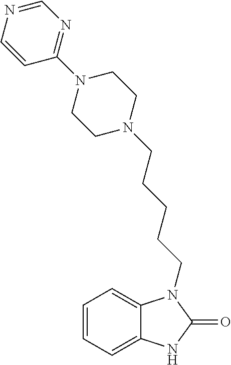 [SMILES: O=c1[nH]c2ccccc2n1CCCCCN1CCN(c2ccncn2)CC1]

The reaction was performed according to general procedure A. Beige solid (110 mg, 57%).

¹H NMR (400 MHz, CDCl₃) δ 9.39 (s, 1H), 8.58 (s, 1H), 8.18 (d, J=6.3 Hz, 1H), 7.13-7.03 (m, 3H), 6.99 (dd, J=7.7, 1.9 Hz, 1H), 6.46 (dd, J=6.3, 1.2 Hz, 1H), 3.90 (t, J=7.3 Hz, 2H), 3.63 (t, J=5.0 Hz, 4H), 2.48 (t, J=5.0 Hz, 4H), 2.37 (t, J=7.3 Hz, 2H), 1.81 (p, J=7.3 Hz, 2H), 1.58 (p, J=7.3 Hz, 2H), 1.42 (p, J=7.3 Hz, 2H).

¹³C NMR (101 MHz, CDCl₃) δ 161.40, 158.50, 155.67, 155.46, 130.54, 127.99, 121.55, 121.45, 109.61, 108.00, 103.06, 58.44, 52.85, 43.72, 40.85, 28.33, 26.48, 24.80.

HRMS (ESI) m/z: calculated for C₂₀H₇N₆O [M+H]⁺ 367.2241; found 367.2245

m.p.: 131° C.

### 1-(5-(4-(pyrimidin-2-yl)piperazin-1-yl)pentyl)-1H-benzo[d]imidazol-2(3H)-one

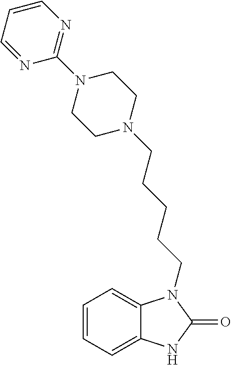 [SMILES: O=c1[nH]c2ccccc2n1CCCCCN1CCN(c2ncccn2)CC1]

The reaction was performed according to general procedure A. Beige solid (131 mg, 68%).

¹H NMR (400 MHz, CDCl₃) δ 9.64 (s, 1H), 8.29 (d, J=4.7 Hz, 2H), 7.13-7.03 (m, 3H), 6.99 (dd, J=7.5, 1.5 Hz, 1H), 6.47 (t, J=4.7 Hz, 1H), 3.90 (t, J=7.4 Hz, 2H), 3.83 (t, J=5.1 Hz, 4H), 2.49 (t, J=5.1 Hz, 4H), 2.38 (t, J=7.4 Hz, 2H), 1.81 (p, J=7.4 Hz, 2H), 1.60 (p, J=7.4 Hz, 2H), 1.43 (p, J=7.4 Hz 2H).

¹³C NMR (101 MHz, CDCl₃) δ 161.78, 157.84, 155.59, 130.53, 128.05, 121.53, 121.40, 109.97, 109.68, 107.98, 58.65, 53.23, 43.69, 40.90, 28.39, 26.51, 24.91.

HRMS (ESI) m/z: calculated for C₂₀H₇N₆O [M+H]⁺ 367.2241; found 367.2244

m.p.: 149° C.

### 1-(5-(4-(1,3,5-triazin-2-yl)piperazin-1-yl)pentyl)-1H-benzo[d]imidazol-2(3H)-one

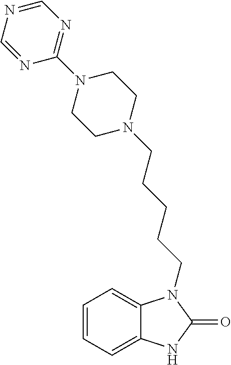 [SMILES: O=c1[nH]c2ccccc2n1CCCCCN1CCN(c2ncncn2)CC1]

The reaction was performed according to general procedure A. Beige solid (80 mg, 41%).

¹H NMR (400 MHz, CDCl₃) δ 9.37 (s, 1H), 8.52 (s, 2H), 7.16-7.01 (m, 3H), 7.04-6.93 (m, 1H), 4.00-3.83 (m, 6H), 2.60 (s, 4H), 2.48 (t, J=7.6 Hz, 2H), 1.81 (p, J=7.6 Hz, 2H), 1.64 (p, J=7.6 Hz, 2H), 1.43 (p, J=7.6 Hz, 2H).

¹³C NMR (101 MHz, CDCl₃) δ 165.76, 162.89, 155.46, 130.41, 127.82, 121.47, 121.36, 109.50, 107.84, 77.53, 77.02, 76.51, 58.06, 52.52, 42.44, 40.56, 29.64, 28.06, 25.75, 24.45.

HRMS (ESI) m/z: calculated for C₁₉H₂₆N₇O [M+H]⁺ 368.2193; found 368.2198

m.p.: 149° C.

### 1-(5-(4-(4-fluorophenyl)piperidin-1-yl)pentyl)-1H-benzo[d]imidazol-2(3H)-one

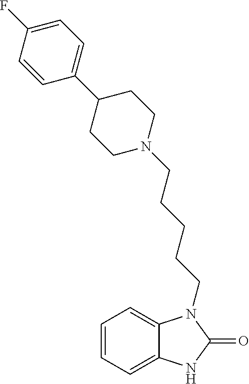 [SMILES: O=c1[nH]c2ccccc2n1CCCCCN1CCC(c2ccc(F)cc2)CC1]

The reaction was performed according to general procedure A. Beige solid (88 mg, 44%).

¹H NMR (400 MHz, CDCl₃) δ 9.68 (s, 1H), 7.20-7.13 (m, 2H), 7.13-7.03 (m, 3H), 7.02-6.91 (m, 3H), 3.90 (t, J=7.1 Hz, 2H), 3.14 (d, J=11.0 Hz, 2H), 2.56-2.42 (m, 3H), 2.14 (td, J=11.0, 3.4 Hz, 2H), 1.94-1.73 (m, 6H), 1.66 (p, J=7.8 Hz, 2H), 1.42 (p, J=7.8 Hz, 2H).

¹³C NMR (101 MHz, CDCl₃) δ 161.54 (d, J=243.9 Hz), 155.59, 141.54, 130.50, 128.31 (d, J=7.8 Hz), 128.07, 121.57, 121.45, 115.30 (d, J=21.0 Hz), 109.70, 108.01, 58.66, 54.24, 41.74, 40.78, 33.06, 29.83, 28.29, 26.14, 24.87.

¹⁹F NMR (356 MHz, CDCl₃) 5-117.03.

HRMS (ESI) m/z: calculated for C₂₃H₉FN₃O [M+H]⁺ 382.2289; found 382.2297

m.p.: 108° C.

### (3R,5S)-1-(4-fluorophenyl)-3,5-dimethylpiperazine

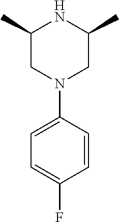

In a sealed tube, 1-bromo-4-fluorobenzene (134 mg, 0.77 mmol, 1 eq.), (3R,5S)-3,5-dimethylpiperazine (350 mg, 4 eq.), Pd₂dba₃ (7 mg, 1 mol %), RuPhos (7 mg, 2 mol %), and t-BuONa (110 mg, 1.5 eq.) were dissolved in dioxane (2.5 mL). The tube was sealed and the mixture was stirred at 100° C. for 10 min. The reaction mixture was filtered on Celite, washed with DCM. The solvents were removed under reduced pressure and the crude was directly purified by flash chromatography (DCM to DCM/MeOH 8%) to obtain the desired product as a brown oil (80 mg, 50%).

¹H NMR (400 MHz, CDCl₃) δ 6.96 (t, J=8.7 Hz, 2H), 6.87 (dd, J=9.1, 4.5 Hz, 2H), 3.39 (dd, J=12.0, 2.7 Hz, 2H), 3.08 (ddd, J=9.8, 6.3, 3.0 Hz, 2H), 2.30 (t, J=11.2 Hz, 2H), 1.16 (d, J=6.3 Hz, 6H).

¹³C NMR (101 MHz, CDCl₃) δ 157.29 (d, J=237.9 Hz), 148.11, 118.17 (d, J=7.6 Hz), 115.65 (d, J=22.1 Hz), 57.36, 50.98, 19.71.

HRMS (ESI) m/z: calculated for C₁₂H₁₈FN₂ [M+H]⁺ 209.1449; found 209.1452

### 1-(5-((2R,6S)-4-(4-fluorophenyl)-2,6-dimethylpiperazin-1-yl)pentyl)-1H-benzo[d]imidazol-2(3H)-one

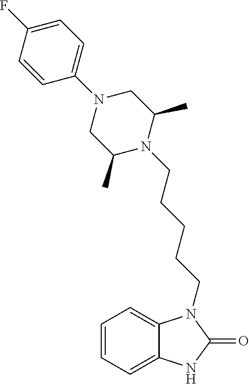 [SMILES: C[C@@H]1CN(c2ccc(F)cc2)C[C@H](C)N1CCCCCn1c(=O)[nH]c2ccccc21]

The reaction was performed according to general procedure A. Brown oil (11 mg, 8%).

¹H NMR (400 MHz, CDCl₃) δ 9.47 (s, 1H), 7.14-7.02 (m, 3H), 7.03-6.96 (m, 1H), 6.99-6.90 (m, 2H), 6.90-6.80 (m, 2H), 3.90 (t, J=7.1 Hz, 2H), 3.36-3.29 (m, 2H), 2.87-2.78 (m, 4H), 1.82 (p, J=7.3 Hz, 2H), 1.52 (p, J=8.7 Hz, 2H), 1.34 (p, J=7.8 Hz, 2H), 1.25 (s, 2H), 1.17 (d, J=6.3 Hz, 6H).

¹³C NMR (101 MHz, CDCl₃) δ 155.53, 147.53, 130.49, 128.00, 121.61, 121.48, 118.15, 115.67 (d, J=22.1 Hz), 109.70, 107.97, 57.70, 54.11, 47.80, 40.78, 29.84, 28.41, 24.84, 17.72.

HRMS (ESI) m/z: calculated for C₂₄H₃₂FN₄O [M+H]⁺ 411.2555; found 411.2563

### tert-butyl-4-(4-fluorophenyl)-1,4-diazepane-1-carboxylate

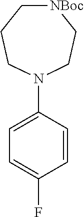 [SMILES: CC(C)(C)OC(=O)N1CCCN(c2ccc(F)cc2)CC1]

The reaction was performed according to general procedure B. Beige solid (83 mg, 56%).

¹H NMR (400 MHz, CDCl₃) δ ¹³C NMR (101 MHz, CDCl₃) δ 6.92 (t, J=8.5 Hz, 2H), 6.61 (dd, J=9.3, 4.2 Hz, 2H), 3.61-3.46 (m, 6H), 3.32 (t, J=6.1 Hz, 1H), 3.21 (t, J=6.2 Hz, 1H), 2.01-1.89 (m, 2H), 1.43-1.36 (m, 9H).

¹³C NMR (101 MHz, CDCl₃) δ 155.49, 155.17, 144.07, 116.12, 115.90, 112.81, 112.73, 112.60, 112.53, 79.65, 50.88, 50.66, 49.29, 48.57, 46.47, 46.39, 46.21, 45.80, 29.85, 28.55, 28.45, 25.47, 25.22.

¹⁹F NMR (356 MHz, CDCl₃) δ −122.89

HRMS (ESI) m/z: calculated for C₁₆H₂₄FN₂O₂[M+H]⁺ 295.1816; found 295.1819

m.p.=85° C.

### 1-(4-fluorophenyl)-1,4-diazepane

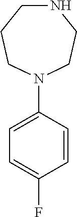 [SMILES: Fc1ccc(N2CCCNCC2)cc1]

The reaction was performed according to general procedure C. Beige solid (76 mg, 88%).

¹H NMR (400 MHz, CDCl₃) δ 6.92 (t, J=8.5 Hz, 2H), 6.66-6.55 (m, 2H), 3.53 (t, J=5.7 Hz, 4H), 3.28 (s, 1H), 3.06 (t, J=5.3 Hz, 2H), 2.88 (t, J=5.7 Hz, 2H), 1.94 (p, J=6.2 Hz, 2H).

¹³C NMR (101 MHz, CDCl₃) δ 155.13 (d, J=234.7 Hz), 145.19 (d, J=1.7 Hz), 115.89 (d, J=22.0 Hz), 112.60 (d, J=7.2 Hz), 51.64, 48.61, 48.13, 47.64, 29.18.

HRMS (ESI) m/z: calculated for C₁₁H₁₆FN₂ [M+H]⁺ 195.1292; found 195.1294

### 1-(5-(4-(5-fluorophenyl)-1,4-diazepan-1-yl)pentyl)-1H-benzo[d]imidazol-2(3H)-one

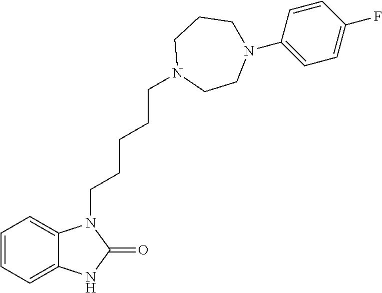 [SMILES: O=c1[nH]c2ccccc2n1CCCCCN1CCCN(c2ccc(F)cc2)CC1]

The reaction was performed according to general procedure A. Orange oil (86 mg, 72%).

¹H NMR (400 MHz, CDCl₃) δ 9.71 (s, 1H), 7.07 (dq, J=13.7, 7.2 Hz, 3H), 6.97 (d, J=7.2 Hz, 1H), 6.91 (t, J=8.5 Hz, 2H), 6.62-6.54 (m, 2H), 3.88 (t, J=7.0 Hz, 2H), 3.59 (t, J=4.8 Hz, 2H), 3.41 (t, J=6.3 Hz, 2H), 2.90 (t, J=4.8 Hz, 2H), 2.78 (t, J=4.8 Hz, 2H), 2.62 (t, J=7.7 Hz, 2H), 2.10 (p, J=6.0 Hz, 2H), 1.79 (p, J=7.3 Hz, 2H), 1.66 (p, J=7.7 Hz, 2H), 1.37 (p, J=7.7 Hz, 2H).

¹³C NMR (101 MHz, CDCl₃) δ 155.57, 155.31 (d, J=235.1 Hz), 145.80 (d, J=1.9 Hz), 130.42, 128.04, 121.60, 121.46, 115.80 (d, J=22.0 Hz), 112.81 (d, J=7.3 Hz), 109.72, 108.00, 57.68, 55.50, 54.41, 48.29, 40.63, 28.11, 26.67, 26.01, 25.50, 24.51.

¹⁹F NMR (356 MHz, CDCl₃) δ −129.32

HRMS (ESI) m/z: calculated for C₂₃H₃₀FN₄O [M+H]⁺ 397.2398; found 397.2401

### tert-butyl-6-(4-fluorophenyl)-2,6-diazaspiro[3.3]heptane-2-carboxylate

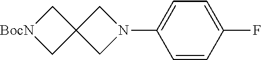 [SMILES: CC(C)(C)OC(=O)N1CC2(C1)CN(c1ccc(F)cc1)C2]

The reaction was performed according to general procedure B. Beige solid (106 mg, 85%).

¹H NMR (400 MHz, CDCl₃) 5 6.97-6.83 (m, 2H), 6.45-6.30 (m, 2H), 4.07 (s, 4H), 3.90 (s, 4H), 1.44 (s, 9H). The spectroscopic data were in agreement with those reported in the literature.

HRMS (ESI) m/z: calculated for C₁₆H₂₂FN₂O₂[M+H]⁺ 293.1660; found 293.1666

### 2-(4-fluorophenyl)-2,6-diazaspiro[3.3]heptane

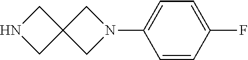 [SMILES: Fc1ccc(N2CC3(CNC3)C2)cc1]

The reaction was performed according to general procedure C. Beige solid (58 mg, 81%).

¹H NMR (400 MHz, CDCl₃) 5 6.97-6.83 (m, 2H), 6.44-6.29 (m, 2H), 3.91 (s, 4H), 3.85 (s, 4H). The spectroscopic data were in agreement with those reported in the literature.

### 1-(5-(6-(4-fluorophenyl)-2,6-diazaspiro[3.3]heptan-2-yl)pentyl)-1H-benzo[d]imidazol-2(3H)-one

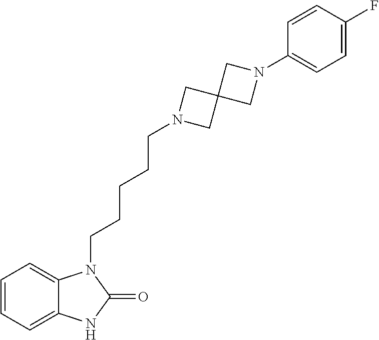 [SMILES: O=c1[nH]c2ccccc2n1CCCCCN1CC2(C1)CN(c1ccc(F)cc1)C2]

The reaction was performed according to general procedure A. Brown oil (14 mg, 14%).

¹H NMR (400 MHz, DMSO-d6) δ 10.83 (s, 1H), 7.12 (d, J=6.7 Hz, 1H), 7.07-6.93 (m, 6H), 6.49-6.40 (m, 1H), 4.32 (dd, J=11.3, 6.1 Hz, 2H), 4.18 (dd, J=11.3, 6.1 Hz, 2H), 3.98 (s, 2H), 3.88 (s, 2H), 3.79 (t, J=6.9 Hz, 2H), 3.11 (q, J=7.5, 7.1 Hz, 2H), 1.70-1.62 (m, 2H), 1.47 (q, J=7.9 Hz, 2H), 1.36-1.20 (m, 2H).

¹³C NMR (101 MHz, DMSO-d6) δ 154.29, 148.07, 130.12, 128.26, 120.78, 120.51, 115.40, 115.18, 114.86, 112.84 (d, J=7.3 Hz), 108.76, 107.73, 62.22, 61.82, 60.48, 53.66, 48.62, 40.44, 33.41, 27.31, 23.60, 22.87.

¹⁹F NMR (356 MHz, CDCl₃) δ −127.50

HRMS (ESI) m/z: calculated for C₂₃H₈FN₄O [M+H]⁺ 395.2242; found 395.2246

### (3aR, 6aS)-tert-butyl-5-(4-fluorophenyl)hexahydropyrrolo[3,4-c]pyrrole-2(1H)-carboxylate

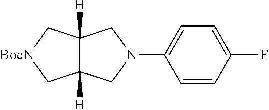 [SMILES: CC(C)(C)OC(=O)N1C[C@@H]2CN(c3ccc(F)cc3)C[C@@H]2C1]

The reaction was performed according to general procedure B. Beige solid (99 mg, 28%).

¹H NMR (400 MHz, CDCl₃) δ 6.99-6.89 (m, 2H), 6.51-6.42 (m, 2H) 3.65 (t, J=8.5 Hz, 2H), 3.48 (s, 2H), 3.41-3.33 (m, 1H), 3.29-31 (m, 1H), 3.17 (dd, J=9.4, 3.6 Hz, 2H), 3.04-2.94 (m, 2H), 1.45 (s, 9H).

¹³C NMR (101 MHz, CDCl₃) δ 155.39 (d, J=234.4 Hz), 154.64, 144.56, 115.73 (d, J=22.2 Hz), 112.69 (d, J=7.3 Hz), 79.58, 52.79, 41.56, 28.65.

¹⁹F NMR (356 MHz, CDCl₃) δ −129.75

HRMS (ESI) m/z: calculated for C₁₇H₄FN₂O₂[M+H]⁺ 307.1816; found 307.1820

### (3aR, 6aS)-2-(4-fluorophenyl)octahydropyrrolo[3,4-c]pyrrole

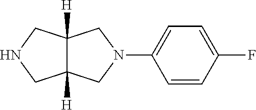 [SMILES: Fc1ccc(N2C[C@H]3CNC[C@H]3C2)cc1]

The reaction was performed according to general procedure C. Beige solid (56 mg, 87%).

¹H NMR (400 MHz, CDCl₃) δ 7.02-6.85 (m, 2H), 6.66-6.50 (m, 2H), 3.59 (s, 1H), 3.35-3.11 (m, 6H), 3.00-2.85 (m, 4H).

### 1-(5-((3aR, 6aS)-5-(4-fluorophenyl)hexahydropyrrolo[3,4-c]pyrrol-2(1H)-yl)pentyl)-1H-benzo[d]imidazol-2(3H)-one

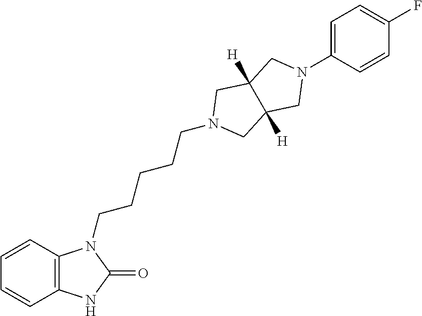 [SMILES: O=c1[nH]c2ccccc2n1CCCCCN1C[C@@H]2CN(c3ccc(F)cc3)C[C@@H]2C1]

The reaction was performed according to general procedure A. Orange oil (35 mg, 43%).

¹H NMR (400 MHz, CDCl₃) δ 8.99 (s, 1H), 7.06 (s, 3H), 7.00-6.87 (m, 3H), 6.63-6.55 (m, 2H), 3.87 (t, J=7.1 Hz, 2H), 3.22-3.16 (m, 4H), 3.03 (s, 3H), 2.57 (t, J=7.8 Hz, 2H), 2.44 (dd, J=9.8, 4.5 Hz, 2H), 1.78 (p, J=7.3 Hz, 2H), 1.65 (p, J=7.9 Hz, 2H), 1.39 (dq, J=14.1, 7.5, 6.9 Hz, 3H).

¹³C NMR (101 MHz, CDCl₃) δ 155.25, 145.56, 130.50, 127.86, 121.55 (d, J=3.6 Hz), 115.65 (d, J=22.0 Hz), 115.10 (d, J=7.4 Hz), 109.55, 108.03, 60.47, 55.37, 54.72, 41.46, 40.65, 28.13, 24.63.

¹⁹F NMR (356 MHz, CDCl₃) δ −127.60

HRMS (ESI) m/z: calculated for C₂₄H₃₀FN₄O [M+H]⁺ 409.2398; found 409.2398

### tert-butyl-3-(2-(2-bromoethoxy)ethyl)-2-oxo-2,3-dihydro-1H-benzo[d]imidazole carboxylate

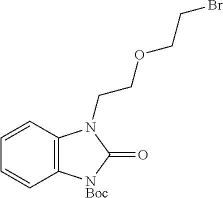 [SMILES: CC(C)(C)OC(=O)n1c(=O)n(CCOCCBr)c2ccccc21]

A solution of tert-butyl-2-oxo-2,3-dihydro-1H-benzo[d]imidazole-1-carboxylate (500 mg, 2.13 mmol, 1 eq.), bis(2-bromoethyl)ether (2.68 mL, 10 eq.), TBAl (39 mg, 0.05 eq.) and K₂CO₃ (2.95 g, 10 eq.) in water (15 mL) was heated at 70° C. overnight. The reaction mixture was extracted with EA. The organic phase was dried over anhydrous MgSO₄, filtered and evaporated under reduced pressure. The crude was purified by flash chromatography (PE to PE/AE 1:1) to afford the desired product as a beige solid (539 mg, 66%).

¹H NMR (400 MHz, CDCl₃) δ 7.81 (d, J=8.3 Hz, 1H), 7.23-7.05 (m, 3H), 4.06 (t, J=5.4 Hz, 2H), 3.81 (t, J=5.4 Hz, 2H), 3.74 (t, J=6.1 Hz, 2H), 3.37 (t, J=6.1 Hz, 2H), 1.68 (s, 9H).

¹³C NMR (101 MHz, CDCl₃) δ 151.32, 149.01, 130.07, 126.24, 124.02, 122.25, 114.44, 108.83, 84.86, 71.16, 69.03, 41.38, 30.34, 28.27.

HRMS (ESI) m/z: calculated for C₁₆H₂₂BrN₂O₄[M+H]⁺ 385.0757; found 385.0758

### 1-(2-(2-bromoethoxy)ethyl)-1H-benzo[d]imidazol-2(3H)-one

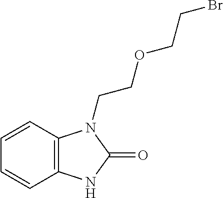 [SMILES: O=c1[nH]c2ccccc2n1CCOCCBr]

To a solution of tert-butyl-3-(2-(2-bromoethoxy)ethyl)-2-oxo-2,3-dihydro-1H-benzo[d]imidazole carboxylate (530 mg, 1.38 mmol, 1 eq.) in DCM (2 mL) was added TFA (4 eq., 0.42 mL). The mixture was stirred at rt for 1 h, then quenched with a NaHCO₃ saturated solution. The aqueous phase was extracted with DCM. The organic layer was dried over anhydrous MgSO₄, concentrated under reduced pressure to afford the desired compound without further purification as a beige solid (353 mg, 90%).

¹H NMR (400 MHz, CDCl₃) δ 10.53 (s, 1H), 7.22-6.91 (m, 4H), 4.11 (t, J=5.5 Hz, 2H), 3.83 (t, J=5.5 Hz, 2H), 3.75 (t, J=6.1 Hz, 2H), 3.38 (t, J=6.1 Hz, 2H).

¹³C NMR (101 MHz, CDCl₃) δ 156.01, 130.76, 128.11, 121.70, 121.38, 109.76, 108.87, 71.13, 69.26, 41.06, 30.28.

HRMS (ESI) m/z: calculated for C₁₁H₁₄BrN₂O₂[M+H]⁺ 285.0233; found 285.0237

m.p.=103° C.

### 1-(2-(2-(4-(4-fluorophenyl)piperazin-1-yl)ethyl)-1H-benzo[d]imidazol-2(3H)-one

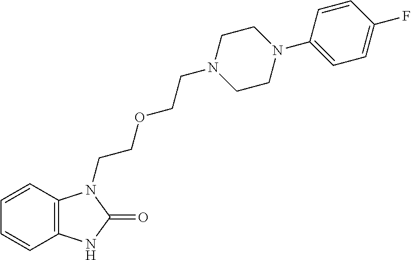 [SMILES: O=c1[nH]c2ccccc2n1CCOCCN1CCN(c2ccc(F)cc2)CC1]

The reaction was performed according to general procedure. Beige solid (66 mg, 50%).

¹H NMR (400 MHz, CDCl₃) δ 10.35 (s, 1H), 7.14-7.00 (m, 4H), 6.98-6.87 (m, 2H), 6.85-6.75 (m, 2H), 4.08 (t, J=5.5 Hz, 2H), 3.78 (t, J=5.5 Hz, 2H), 3.61 (t, J=5.5 Hz, 2H), 3.04-2.97 (m, 4H), 2.61-2.52 (m, 6H).

¹³C NMR (101 MHz, CDCl₃) δ 157.21 (d, J=238.7 Hz), 155.89, 148.05 (d, J=2.2 Hz), 130.84, 128.08, 121.61, 121.33, 117.81 (d, J=7.6 Hz), 115.56 (d, J=22.1 Hz), 109.65, 108.79, 69.39, 68.98, 57.83, 53.64, 50.02, 41.09.

¹⁹F NMR (356 MHz, CDCl₃) δ −124.77

HRMS (ESI) m/z: calculated for C₂₁H₂₆FN₄O₂[M+H]⁺ 385.2034; found 385.2037

m.p.=149° C.

### 3,3-dimethylpentane-1,5-diol

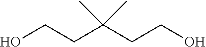

In a round bottomed flask under argon, LiAlH₄ (1.40 g, 2 eq.) was suspended in dry THF (60 mL). A solution of 3,3-dimethylglutaric acid (2.957 g, 18.5 mmol, 1 eq.) in solution in THF (30 mL) was added dropwise. The solution was refluxed for 24 h. The solution was cooled to 0° C., quenched with NaOH 1 M and extracted with EA. The organic phase was dried over anhydrous MgSO₄, filtered and concentrated under reduced pressure to afford the desired compound as a colorless oil (2.32 g, 95%) without further purification.

¹H NMR (400 MHz, CDCl₃) δ 3.74 (t, J=7.1 Hz, 4H), 1.58 (t, J=7.1 Hz, 4H), 0.95 (s, 6H).

### 1,5-dibromo-3,3-dimethylpentane

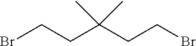

PBr₃ (3.27 mL, 2.2 eq.) was added to 3,3-dimethylpentane-1,5-diol (2.07 g, 15.7 mmol, 1 eq.) in an ice bath. The solution was then heated at 100° C. for 3 h. The reaction mixture was poured on ice and extracted with DCM. The organic phase was washed with NaOH 1M, brine, dried over anhydrous MgSO₄, filtered and evaporated under reduced pressure to afford the desired compound as a colorless oil (2.64 g, 65%).

¹H NMR (400 MHz, CDCl₃) b 3.43-3.29 (m, 4H), 1.93-1.79 (m, 4H), 0.94 (s, 6H).

### tert-butyl-3-(5-bromo-3,3-dimethylpentyl)-2-oxo-2,3-dihydro-1H-benzo[d]imidazole-1-carboxylate

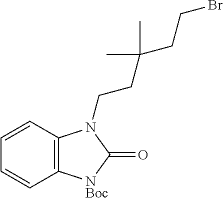 [SMILES: CC(C)(CCBr)CCn1c(=O)n(C(=O)OC(C)(C)C)c2ccccc21]

A solution of tert-butyl-2-oxo-2,3-dihydro-1H-benzo[d]imidazole-1-carboxylate (281 mg, 1.20 mmol, 1 eq.), 1,5-dibromo-3,3-dimethylpentane (1.55 g, 5 eq.), TBAl (22 mg, 0.05 eq.) and K₂CO₃ (1.66 g, 10 eq.) in water (9 mL) was heated at 70° C. for 3 h. The reaction mixture was extracted with EA. The organic phase was dried over anhydrous MgSO₄, filtered and evaporated under reduced pressure. The crude was purified by flash chromatography (PE to PE/AE 10%) to afford the desired product as a colorless oil (295 mg, 60%).

¹H NMR (400 MHz, CDCl₃) δ 7.84 (dd, J=7.9, 1.2 Hz, 1H), 7.20 (td, J=7.9, 1.2 Hz, 1H), 7.12 (td, J=7.9, 1.3 Hz, 1H), 6.91 (dd, J=7.9, 1.2 Hz, 1H), 3.89-3.80 (m, 2H), 3.45-3.36 (m, 2H), 1.99-1.90 (m, 2H), 1.67 (s, 9H), 1.66-1.57 (m, 2H), 1.04 (s, 6H).

¹³C NMR (101 MHz, CDCl₃) δ 150.91, 149.04, 129.26, 126.51, 124.04, 122.24, 114.78, 107.32, 84.84, 51.01, 45.71, 38.84, 37.11, 33.93, 28.63, 28.25, 26.69.

HRMS (ESI) m/z: calculated for C₁₉H₇BrN₂O₃Na [M+Na]⁺ 433.1097; found 433.1099

### 1-(5-bromo-3,3-dimethylpentyl)-1H-benzo[d]imidazol-2(3H)-one

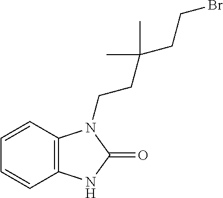 [SMILES: CC(C)(CCBr)CCn1c(=O)[nH]c2ccccc21]

To a solution of tert-butyl-3-(5-bromo-3,3-dimethylpentyl)-2-oxo-2,3-dihydro-1H-benzo[d]imidazole-1-carboxylate (282 mg, 0.69 mmol, 1 eq.) in DCM (1 mL) was added TFA (0.21 mL, 4 eq.). The mixture was stirred at rt for 15 min, then quenched with a NaHCO₃ saturated solution. The aqueous phase was extracted with DCM. The organic layer was dried over anhydrous MgSO₄, concentrated under reduced pressure to afford the desired compound without further purification as a white solid (199 mg, 93%).

¹H NMR (400 MHz, CDCl₃) δ 9.81 (s, 1H), 7.15-7.11 (m, 1H), 7.12-7.03 (m, 2H), 6.97-6.94 (m, 1H), 3.94-3.85 (m, 2H), 3.48-3.39 (m, 2H), 2.02-1.93 (m, 2H), 1.71-1.64 (m, 2H), 1.06 (s, 6H).

¹³C NMR (101 MHz, CDCl₃) δ 155.40, 130.15, 128.19, 121.65, 121.45, 109.92, 107.70, 45.72, 39.40, 36.89, 34.01, 28.79, 26.75.

HRMS (ESI) m/z: calculated for C₁₄H₂₀BrN₂O [M+H]⁺ 311.0754; found 311.0758

### 1-(5-(4-(4-fluorophenyl)piperazin-1-yl)-3,3-dimethylpentyl)-1H-benzo[d]imidazol-2(3H)-one

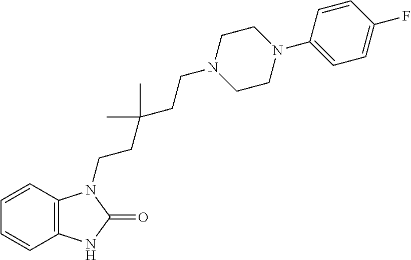 [SMILES: CC(C)(CCN1CCN(c2ccc(F)cc2)CC1)CCn1c(=O)[nH]c2ccccc21]

The reaction was performed according to general procedure A. Beige solid (183 mg, 73%).

¹H NMR (400 MHz, CDCl₃) δ 9.37 (s, 1H), 7.13-7.01 (m, 3H), 7.00-6.92 (m, 3H), 6.90-6.84 (m, 2H), 3.95-3.86 (m, 2H), 3.14 (dd, J=6.2, 3.6 Hz, 4H), 2.64 (dd, J=6.2, 3.6 Hz, 4H), 2.50-2.41 (m, 2H), 1.73-1.64 (m, 2H), 1.63-1.55 (m, 2H), 1.06 (s, 6H).

¹³C NMR (101 MHz, CDCl₃) δ 157.31 (d, J=239.0 Hz), 155.19, 148.12, 130.35, 128.07, 121.53, 121.43, 117.92 (d, J=7.6 Hz), 115.63 (d, J=22.1 Hz), 109.67, 107.84, 54.17, 53.66, 50.28, 39.70, 38.51, 37.11, 32.00, 27.25.

¹⁹F NMR (356 MHz, CDCl₃) δ −124.58.

HRMS (ESI) m/z: calculated for C₂₄H₃₂FN₄O [M+H]⁺ 411.2555; found 411.2554

m.p.=158° C.

### tert-butyl-2-oxo-2,3-dihydro-1H-imidazole-1-carboxylate

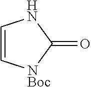 [SMILES: CC(C)(C)OC(=O)n1cc[nH]c1=O]

In a round-bottomed flask under argon, 1H-imidazol-2(3H)-one (399 mg, 4 eq.) was dissolved in dry DMF (15 mL). NaH (190 mg, 4 eq.) was added portionwise and the mixture was stirred for 1 h at room temperature. Boc₂O (259 mg, 1.199 mmol, 1 eq.) was dissolved in 5 mL of DMF and added dropwise to the reaction mixture. The mixture was stirred at room temperature for 24 h. The DMF was removed under reduced pressure. The residue was dissolved in water, extracted with EtOAc. The organic layers were dried on anhydrous MgSO4 and concentrated under reduced pressure. The crude was purified by flash chromatography (PE to EtOAC) to afford the desired product as a white solid (166 mg, 76%)

¹H NMR (400 MHz, DMSO-d6) δ 10.26 (s, 1H), 6.60 (dd, J=3.3, 1.5 Hz, 1H), 6.49 (dd, J=3.3, 1.5 Hz, 1H), 1.49 (s, 9H).

¹³C NMR (101 MHz, DMSO-d6) δ 150.69, 147.53, 110.81, 108.28, 82.83, 27.55.

HRMS (ESI) m/z: calculated for C₃H₁₃N₂O₃ [M+H]⁺ 185.0921; found 185.0917

m.p.=120° C.

### tert-butyl-3-(5-bromopentyl)-2-oxo-2,3-dihydro-1H-imidazole-1-carboxylate

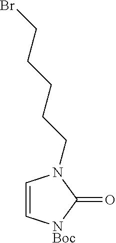 [SMILES: CC(C)(C)OC(=O)n1ccn(CCCCCBr)c1=O]

tert-butyl-2-oxo-2,3-dihydro-1H-imidazole-1-carboxylate (156 mg, 0.85 mmol, 1 eq.), 1,5-dibromopentane (0.51 mL, 5 eq.), TBAl (16 mg, 0.05 eq.) and K₂CO₃ (1.17 g, 10 eq) were dissolved in water (6 mL). The solution was stirred at 70° C. for 1 h. The mixture was extracted with EtOAc. The organic layers were dried on anhydrous MgSO4 and concentrated under reduced pressure. The crude was purified by flash chromatography (PE to PE/EtOAC 7:3) to afford the desired product as a beige solid (100 mg, 35%).

¹H NMR (400 MHz, CDCl₃) δ 6.66 (d, J=3.3 Hz, 1H), 6.17 (d, J=3.3 Hz, 1H), 3.58 (t, J=7.1 Hz, 2H), 3.40 (t, J=6.7 Hz, 2H), 1.89 (dt, J=14.5, 6.8 Hz, 2H), 1.69 (p, J=7.5 Hz, 2H), 1.59 (s, 9H), 1.53-1.41 (m, 2H).

¹³C NMR (101 MHz, CDCl₃) δ 112.56, 107.89, 84.36, 43.38, 33.51, 32.30, 28.33, 28.11, 25.24.

HRMS (ESI) m/z: calculated for C₃H₁₄BrN₂O [M+H]⁺ 233.0284; found 233.0285: Boc-deprotected molecule. The Boc-protected molecule could not be observed.

m.p.=120° C.

### tert-butyl-3-(5-(4-(4-fluorophenyl)piperazin-1-yl)pentyl)-2-oxo-2,3-dihydro-1H-imidazole-1-carboxylate

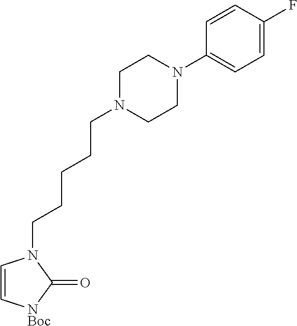 [SMILES: CC(C)(C)OC(=O)n1ccn(CCCCCN2CCN(c3ccc(F)cc3)CC2)c1=O]

The reaction was performed according to general procedure A. Beige solid (96 mg, 74%).

¹H NMR (400 MHz, CDCl₃) δ 6.98-6.89 (m, 2H), 6.89-6.81 (m, 2H), 6.64 (d, J=3.3 Hz, 1H), 6.17 (d, J=3.3 Hz, 1H), 3.56 (t, J=7.2 Hz, 2H), 3.14-3.04 (m, 4H), 2.61-2.54 (m, 3H), 2.41-2.33 (m, 2H), 1.68 (p, J=7.4 Hz, 2H), 1.57 (s, 10H), 1.56-1.48 (m, 1H), 1.34 (p, J=7.5, 6.8 Hz, 2H).

¹³C NMR (101 MHz, CDCl₃) δ 157.24 (d, J=238.6 Hz), 150.53, 148.10, 148.06 (d, J=2.3 Hz), 117.87 (d, J=7.6 Hz), 115.57 (d, J=22.1 Hz), 112.56, 107.74, 84.25, 58.40, 53.33, 50.19, 43.49, 29.03, 28.06, 26.47, 24.60.

HRMS (ESI) m/z: calculated for C₂₃H₃₄FN₄O₃[M+H]⁺ 433.2609; found 433.2616

### 1-(5-(4-(4-fluorophenyl)piperazin-1-yl)pentyl)-1H-imidazol-2(3H)-one

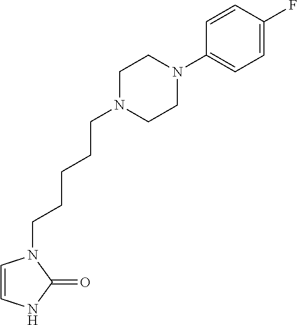 [SMILES: O=c1[nH]ccn1CCCCCN1CCN(c2ccc(F)cc2)CC1]

tert-butyl-3-(5-(4-(4-fluorophenyl)piperazin-1-yl)pentyl)-2-oxo-2,3-dihydro-1H-imidazole-1-carboxylate (96 mg, 0.22 mmol, 1 eq.) was dissolved in DCM (1 mL). TFA (0.1 mL, 7 eq.) was added dropwise. The solution was stirred at room temperature for 1 h, then quenched with saturated NaHCO₃ solution and extracted with DCM. The organic layers were dried on anhydrous MgSO4 and concentrated under reduced pressure to afford the desired product without further purification as a orange solid (66 mg, 89%).

¹H NMR (400 MHz, CDCl₃) δ 10.46 (s, 1H), 6.98-6.91 (m, 2H), 6.91-6.82 (m, 2H), 6.28 (t, J=2.6 Hz, 1H), 6.17 (t, J=2.6 Hz, 1H), 3.61 (t, J=7.4 Hz, 2H), 3.15-3.07 (m, 4H), 2.61 (t, J=5.0 Hz, 4H), 2.44-2.36 (m, 2H), 1.71 (p, J=7.4 Hz, 2H), 1.57 (p, J=7.4 Hz, 2H), 1.37 (p, J=7.4 Hz, 2H).

¹³C NMR (101 MHz, CDCl₃) δ 157.32 (d, J=238.7 Hz), 154.84, 148.06 (d, J=2.2 Hz), 117.95 (d, J=7.6 Hz), 115.62 (d, J=22.0 Hz), 111.41, 108.28, 58.46, 53.31, 50.16, 43.14, 29.62, 26.41, 24.65.

¹⁹F NMR (356 MHz, CDCl₃) δ −124.64

HRMS (ESI) m/z: calculated for C₁₈H₂₆FN₄O [M+H]⁺ 333.2085; found 333.2090

m.p.=118° C.

### tert-butyl-4-(p-tolyl)-1,4-diazepane-1-carboxylate

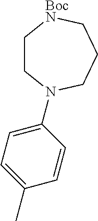 [SMILES: Cc1ccc(N2CCCN(C(=O)OC(C)(C)C)CC2)cc1]

The reaction was performed according to general procedure B. Beige solid (119 mg, 82%).

¹H NMR (400 MHz, CDCl₃) δ 7.02 (d, J=8.2 Hz, 2H), 6.62 (d, J=8.2 Hz, 2H), 3.61-3.48 (m, 6H), 3.30 (t, J=6.2 Hz, 1H), 3.20 (t, J=6.2 Hz, 1H), 2.24 (s, 3H), 1.97 (h, J=6.2 Hz, 2H), 1.48-1.35 (m, 9H). Mixture of conformers.

¹³C NMR (101 MHz, CDCl₃) δ 155.56, 155.21, 145.23, 145.06, 130.18, 130.11, 125.45, 125.34, 112.04, 111.80, 79.54, 50.79, 50.60, 48.68, 47.98, 46.77, 46.13, 45.80, 28.57, 28.46, 25.48, 25.16, 20.28. Mixture of conformers.

HRMS (ESI) m/z: calculated for C₁₇H₇N₂O₂ [M+H]⁺ 291.2067; found 291.2066

### 1-(p-tolyl)-1,4-diazepane

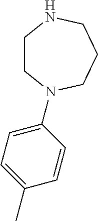

The reaction was performed according to general procedure C. Beige solid (68 mg, 87%).

¹H NMR (400 MHz, CDCl₃) δ 7.06-6.99 (m, 2H), 6.65-6.59 (m, 2H), 3.59-3.51 (m, 4H), 3.08-3.00 (m, 2H), 2.89-2.81 (m, 2H), 2.25 (s, 3H), 1.92 (tt, J=7.1, 4.9 Hz, 2H)

¹³C NMR (101 MHz, CDCl₃) δ 146.28, 130.06, 125.12, 111.78, 51.62, 48.39, 48.18, 47.74, 29.58, 20.27.

HRMS (ESI) m/z: calculated for C₁₂H₁₉N₂[M+H]⁺ 191.1543; found 191.1546

### 1-(5-(4-(p-tolyl)-1,4-diazepan-1-yl)pentyl)-1H-benzo[d]imidazol-2(3H)-one

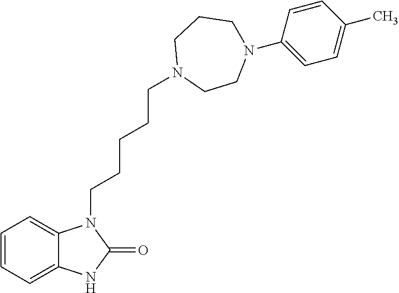 [SMILES: Cc1ccc(N2CCCN(CCCCCn3c(=O)[nH]c4ccccc43)CC2)cc1]

The reaction was performed according to general procedure A. Beige solid (78 mg, 67%).

¹H NMR (400 MHz, CDCl₃) δ 10.02 (s, 1H), 7.14-6.93 (m, 6H), 6.63-6.55 (m, 2H), 3.88 (t, J=7.1 Hz, 2H), 3.56 (dd, J=5.7, 3.8 Hz, 2H), 3.44 (t, J=6.3 Hz, 2H), 2.87-2.80 (m, 2H), 2.70 (dd, J=6.9, 4.0 Hz, 2H), 2.59-2.51 (m, 2H), 2.24 (s, 3H), 2.03 (p, J=6.0 Hz, 2H), 1.79 (p, J=7.3 Hz, 2H), 1.60 (p, J=7.5 Hz, 1H), 1.38 (p, J=7.6, 6.9 Hz, 2H).

¹³C NMR (101 MHz, CDCl₃) δ 155.72, 147.01, 130.43, 129.95, 128.14, 125.34, 121.53, 121.35, 111.88, 109.76, 107.95, 57.57, 55.50, 54.43, 48.29, 48.02, 40.74, 28.24, 27.11, 26.44, 24.66, 20.29.

HRMS (ESI) m/z: calculated for C₂₄H₃₃N₄O [M+H]⁺ 393.2649; found 393.2656

### tert-butyl-4-(pyridin-2-yl)-1,4-diazepane-1-carboxylate

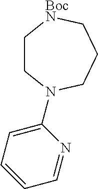 [SMILES: CC(C)(C)OC(=O)N1CCCN(c2ccccn2)CC1]

The reaction was performed according to general procedure B. Beige solid (120 mg, 87%).

¹H NMR (400 MHz, CDCl₃) δ 8.13 (ddd, J=5.0, 2.0, 0.9 Hz, 1H), 7.42 (ddd, J=8.9, 7.1, 2.0 Hz, 1H), 6.56-6.46 (m, 2H), 3.81-3.73 (m, 2H), 3.70-3.58 (m, 2H), 3.59-3.53 (m, 2H), 3.34 (t, J=6.1 Hz, 1H), 3.24 (t, J=6.1 Hz, 1H), 1.96 (p, J=6.1 Hz, 2H), 1.47-1.35 (m, 9H). Mixture of conformers.

¹³C NMR (101 MHz, CDCl₃) δ 148.40, 137.50, 111.90, 105.71, 79.59, 49.07, 48.71, 47.51, 47.26, 46.78, 46.37, 45.98, 28.56, 28.49, 25.52. Mixture of conformers.

HRMS (ESI) m/z: calculated for C₁₅H₄N₃O₂ [M+H]⁺ 278.1863; found 278.1864

### 1-(pyridin-2-yl)-1,4-diazepane

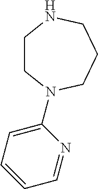 [SMILES: c1ccc(N2CCCNCC2)nc1]

The reaction was performed according to general procedure C. Beige solid (47 mg, 61%).

¹H NMR (400 MHz, CDCl₃) δ 8.12 (dd, J=5.2, 2.0 Hz, 1H), 7.41 (ddd, J=8.9, 7.0, 2.0 Hz, 1H), 6.54-6.44 (m, 2H), 3.76-3.72 (m, 2H), 3.70 (t, J=6.2 Hz, 2H), 3.07-2.99 (m, 2H), 2.90-2.81 (m, 2H), 2.54 (s, 1H), 1.90 (p, J=5.8 Hz, 2H).

¹³C NMR (101 MHz, CDCl₃) δ 158.09, 148.23, 137.42, 111.59, 105.59, 49.78, 48.79, 48.10, 46.65, 29.63.

HRMS (ESI) m/z: calculated for C₁₀H₁₆N₃ [M+H]⁺ 178.1339; found 178.1340 1-(5-(4-(pyridin-2-yl)-1,4-diazepan-1-yl)pentyl)-1H-benzo[d]imidazol-2(3H)-one

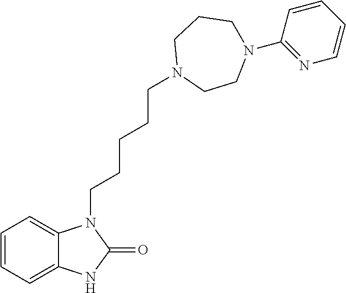 [SMILES: O=c1[nH]c2ccccc2n1CCCCCN1CCCN(c2ccccn2)CC1]

The reaction was performed according to general procedure A. Beige solid (56 mg, 70%).

¹H NMR (400 MHz, CDCl₃) δ 9.94 (s, 1H), 8.12 (dd, J=5.1, 1.9 Hz, 1H), 7.42 (ddd, J=8.8, 7.1, 2.0 Hz, 1H), 7.14-7.00 (m, 3H), 6.96 (dd, J=7.4, 1.6 Hz, 1H), 6.52 (dd, J=7.1, 4.9 Hz, 1H), 6.46 (d, J=8.6 Hz, 1H), 3.87 (t, J=7.0 Hz, 4H), 3.59 (t, J=6.3 Hz, 2H), 2.89 (t, J=4.7 Hz, 2H), 2.75 (dd, J=7.2, 3.8 Hz, 2H), 2.63-2.54 (m, 2H), 2.10 (p, J=5.9 Hz, 2H), 1.78 (p, J=7.3 Hz, 2H), 1.63 (p, J=7.6 Hz, 2H), 1.37 (p, J=7.8 Hz, 1H).

¹³C NMR (101 MHz, CDCl₃) δ 158.22, 155.65, 148.06, 137.52, 130.41, 128.11, 121.55, 121.38, 111.92, 109.74, 107.95, 105.63, 57.70, 55.85, 54.75, 46.30, 45.25, 40.67, 28.17, 26.67, 26.12, 24.58.

HRMS (ESI) m/z: calculated for C₂₄H₃₀N₅O [M+H]⁺ 380.2445; found 380.2448

### tert-butyl-6-(p-tolyl)-2,6-diazaspiro[3.3]heptane-2-carboxylate

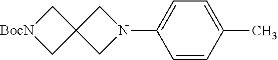 [SMILES: Cc1ccc(N2CC3(CN(C(=O)OC(C)(C)C)C3)C2)cc1]

The reaction was performed according to general procedure B. Beige solid (90 mg, 68%).

¹H NMR (400 MHz, CDCl₃) δ 7.06-6.99 (m, 2H), 6.43-6.35 (m, 2H), 4.07 (s, 4H), 3.92 (s, 4H), 2.25 (s, 3H), 1.44 (s, 9H).

¹³C NMR (101 MHz, CDCl₃) δ 156.22, 149.46, 129.66, 127.58, 112.06, 79.83, 62.65, 33.66, 28.53, 20.60.

HRMS (ESI) m/z: calculated for C₁₇H₅N₂O₂ [M+H]⁺ 289.1911; found 289.1916

### 2-(p-tolyl)-2,6-diazaspiro[3.3]heptane

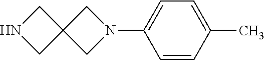

The reaction was performed according to general procedure C. Beige solid 0.

¹H NMR (400 MHz, CDCl₃) δ 7.08-6.98 (m, 2H), 6.43-6.35 (m, 2H), 5.30 (br s, 1H), 3.94 (s, 8H), 2.25 (s, 3H).

¹³C NMR (101 MHz, CDCl₃) δ 149.42, 129.64, 127.53, 112.06, 62.71, 57.03, 20.58.

HRMS (ESI) m/z: calculated for C₁₂H₁₇N₂[M+H]⁺ 189.1386; found 189.1390

### 1-(5-(6-(p-tolyl)-2,6-diazaspiro[3.3]heptan-2-yl)pentyl)-1H-benzo[d]imidazol-2(3H)-one

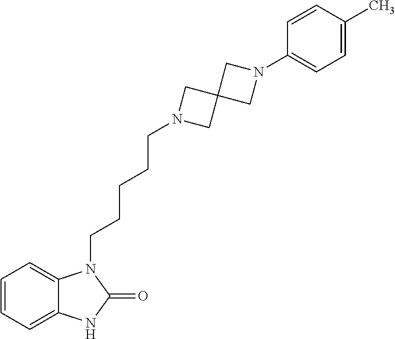 [SMILES: Cc1ccc(N2CC3(CN(CCCCCn4c(=O)[nH]c5ccccc54)C3)C2)cc1]

The reaction was performed according to general procedure A. Yellow oil (50 mg, 39%).

¹H NMR (400 MHz, CDCl₃) δ 10.09 (s, 1H), 7.10-7.03 (m, 3H), 7.02-6.94 (m, 3H), 6.34 (d, J=8.4 Hz, 2H), 3.97-3.81 (m, 6H), 3.66 (s, 4H), 2.64 (t, J=7.6 Hz, 2H), 2.24 (s, 3H), 1.77 (p, J=7.2 Hz, 2H), 1.53 (p, J=7.6 Hz, 2H), 1.44-1.32 (m, 2H).

¹³C NMR (101 MHz, CDCl₃) δ 155.68, 149.30, 130.34, 129.60, 128.11, 127.51, 121.60, 121.42, 112.05, 109.75, 107.98, 63.88, 62.21, 57.99, 40.45, 34.52, 28.07, 26.04, 24.23, 20.57.

HRMS (ESI) m/z: calculated for C₂₄H₃₁N₄O [M+H]⁺ 391.2492; found 391.2497

### tert-butyl-6-(pyridin-2-yl)-2,6-diazaspiro[3.3]heptane-2-carboxylate

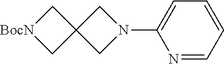 [SMILES: CC(C)(C)OC(=O)N1CC2(C1)CN(c1ccccn1)C2]

The reaction was performed according to general procedure B. Beige solid (96 mg, 82%).

¹H NMR (400 MHz, CDCl₃) δ 8.14 (ddd, J=5.1, 1.9, 0.9 Hz, 1H), 7.45 (ddd, J=8.3, 7.2, 1.9 Hz, 1H), 6.63 (ddd, J=7.2, 5.1, 0.9 Hz, 1H), 6.29 (dt, J=8.3, 0.9 Hz, 1H), 4.11 (d, J=6.4 Hz, 8H), 1.44 (s, 9H).

¹³C NMR (101 MHz, CDCl₃) δ 160.53, 156.26, 148.42, 137.43, 113.65, 106.35, 80.01, 61.07, 33.62, 28.61.

HRMS (ESI) m/z: calculated for C₁₅H₂N₃O₂ [M+H]⁺ 276.1707; found 276.1711

### 2-(pyridin-2-yl)-2,6-diazaspiro[3.3]heptane

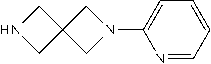 [SMILES: c1ccc(N2CC3(CNC3)C2)nc1]

The reaction was performed according to general procedure C. The aqueous phase was extracted 8 times with EtOAC/MeOH 5%. The desired compound was recovered as on oil in EtOAc and used as such in the next step. Yellow oil.

¹H NMR (400 MHz, MeOD) δ 8.00 (ddd, J=5.2, 1.9, 0.9 Hz, 1H), 7.55 (ddd, J=8.4, 7.2, 1.9 Hz, 1H), 6.68 (ddd, J=7.2, 5.2, 0.9 Hz, 1H), 6.43 (dt, J=8.4, 0.9 Hz, 1H), 4.17 (d, J=7.8 Hz, 8H).

HRMS (ESI) m/z: calculated for C₁₀H₁₄N₃[M+H]⁺ 176.1182; found 176.1185

### 1-(5-(6-(pyridin-2-yl)-2,6-diazaspiro[3.3]heptan-2-yl)pentyl)-1H-benzo[d]imidazol-2(3H)-one

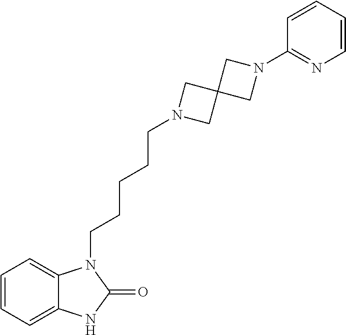 [SMILES: O=c1[nH]c2ccccc2n1CCCCCN1CC2(C1)CN(c1ccccn1)C2]

The reaction was performed according to general procedure A. Yellow oil (8 mg, 7% over 2 steps).

¹H NMR (400 MHz, CDCl₃) δ 9.45 (s, 1H), 8.13 (ddd, J=5.2, 1.9, 0.9 Hz, 1H), 7.43 (ddd, J=8.8, 7.1, 1.9 Hz, 1H), 7.12-7.02 (m, 3H), 6.97 (dd, J=7.9, 1.9 Hz, 1H), 6.60 (ddd, J=7.1, 5.2, 0.9 Hz, 1H), 6.26 (dd, J=8.4, 0.9 Hz, 1H), 4.07 (s, 4H), 3.87 (t, J=7.2 Hz, 2H), 3.47 (s, 4H), 2.48 (t, J=7.1 Hz, 2H), 1.77 (p, J=7.1 Hz, 2H), 1.51-1.30 (m, 4H).

¹³C NMR (101 MHz, CDCl₃) δ 160.50, 155.45, 148.23, 137.24, 130.50, 128.00, 121.55, 121.45, 113.20, 109.61, 107.99, 106.24, 64.36, 60.78, 59.04, 40.73, 34.63, 28.29, 26.87, 24.55.

HRMS (ESI) m/z: calculated for C₂₂H₈N₅O [M+H]⁺ 378.2288; found 378.2291

### Results

### 1. Pharmacology of New Synthesized Compounds

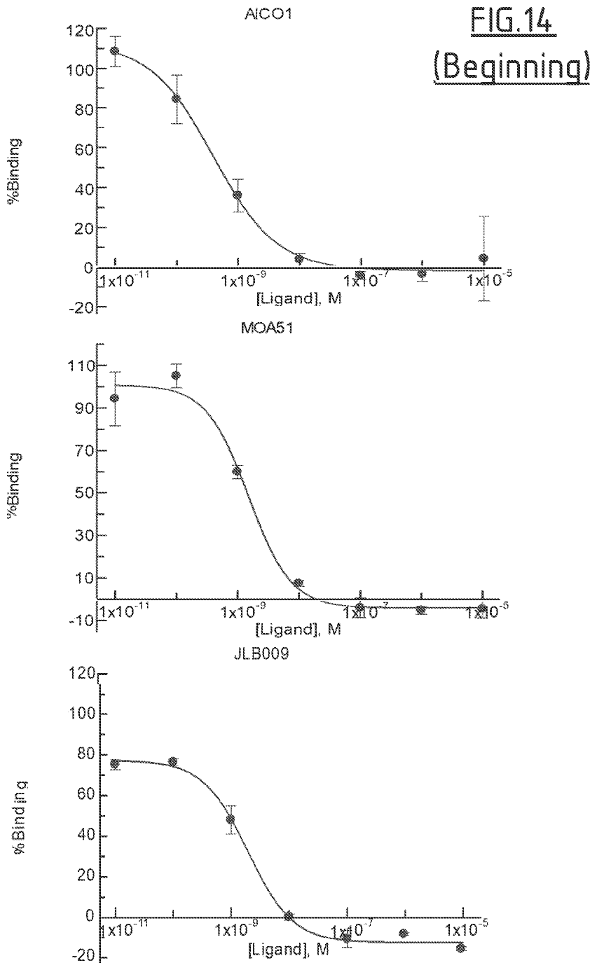

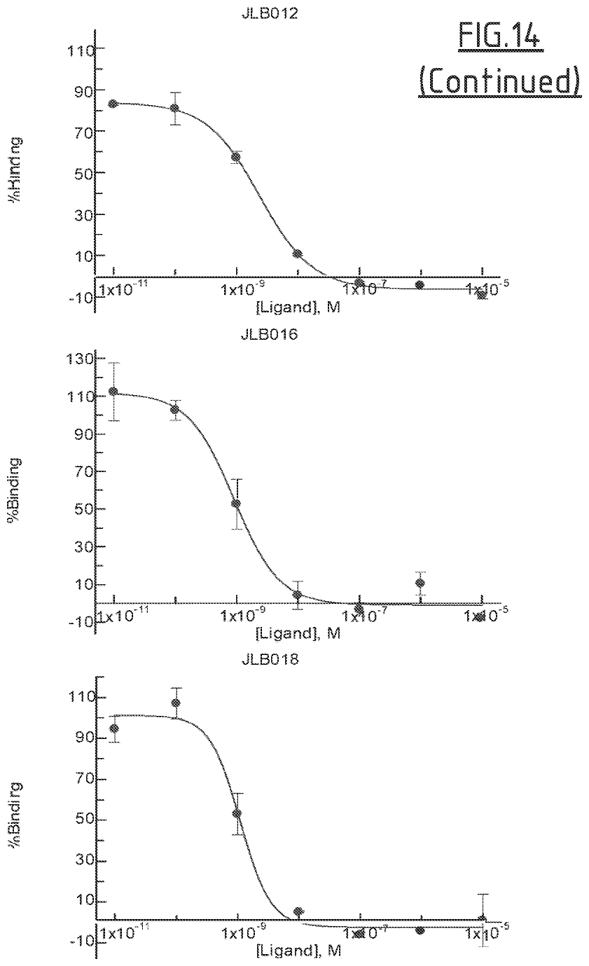

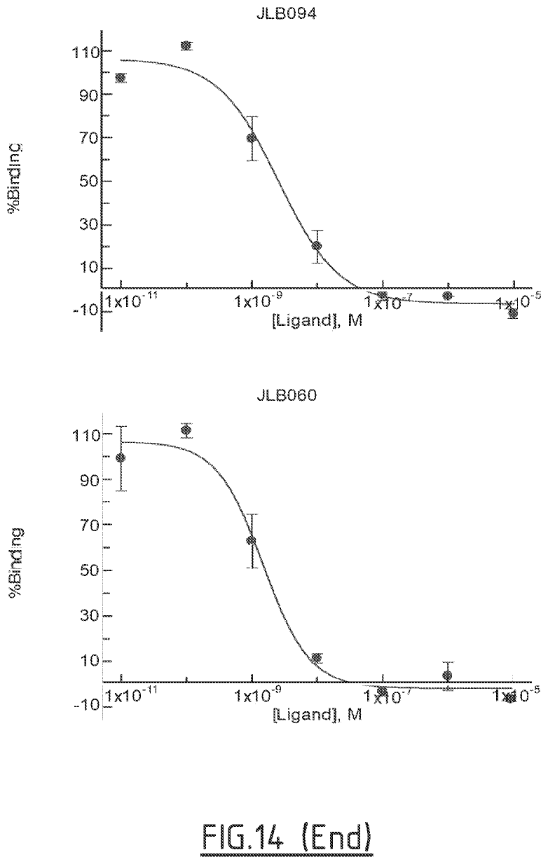

Affinity of new synthesized compounds have been evaluated by binding assays and results have been described in FIG. 14. For the best compounds, their affinity was tested for 5-HT1A, 5-HT2A, 5-HT2Cedited, 5-HT6, 5-HT7 and D2 (long) to evaluate their selectivity (FIG. 14). The table below describes affinity of compounds on 5-HT7 receptor.

| Reference | Structures | IC₅₀ (nM) | Kᵢ (nM) |
|---|---|---|---|
| MOA51 | 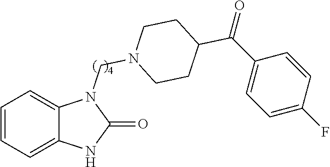 [SMILES: O=C(c1ccc(F)cc1)C1CCN(Cn2c(=O)[nH]c3ccccc32)CC1] | 0.29 | 0.20 |
| Serodolin | 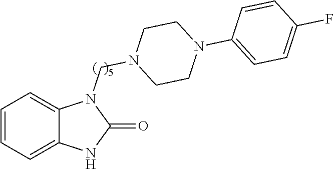 [SMILES: O=c1[nH]c2ccccc2n1CN1CCN(c2ccc(F)cc2)CC1] | 1.14 | 0.8 |
| MOA43 | 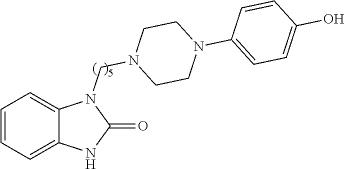 [SMILES: O=c1[nH]c2ccccc2n1CN1CCN(c2ccc(O)cc2)CC1] | 1.95 | 1.37 |
| JLB010 | 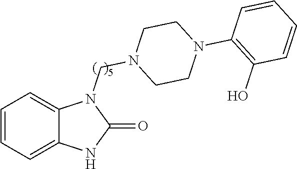 [SMILES: O=c1[nH]c2ccccc2n1CN1CCN(c2ccccc2O)CC1] | 7.99 | 5.61 |
| JLB011 | 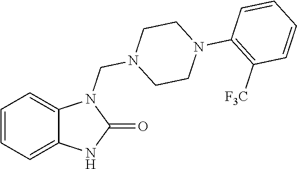 [SMILES: O=c1[nH]c2ccccc2n1CN1CCN(c2ccccc2C(F)(F)F)CC1] | 4.74 | 3.33 |
| JLB012 | 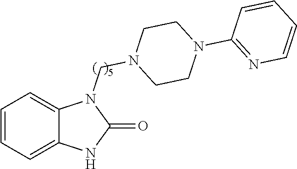 [SMILES: O=c1[nH]c2ccccc2n1CN1CCN(c2ccccn2)CC1] | 1.69 | 1.18 |
| JLB013 | 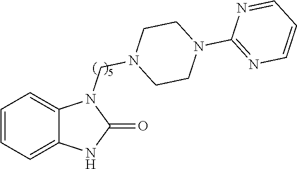 [SMILES: O=c1[nH]c2ccccc2n1CN1CCN(c2ncccn2)CC1] | 9.68 | 6.80 |
| JLB014 | 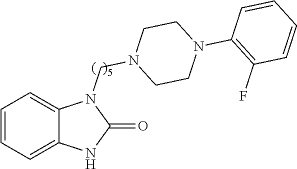 [SMILES: O=c1[nH]c2ccccc2n1CN1CCN(c2ccccc2F)CC1] | 3.29 | 2.31 |
| JLB015 | 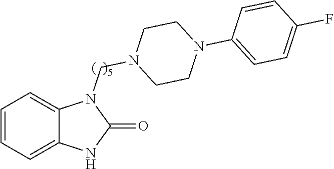 [SMILES: O=c1[nH]c2ccccc2n1CN1CCN(c2ccc(F)cc2)CC1] | 4.39 | 3.09 |
| JLB016 | 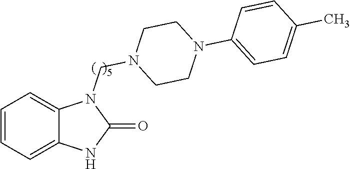 [SMILES: Cc1ccc(N2CCN(Cn3c(=O)[nH]c4ccccc43)CC2)cc1] | 0.61 | 0.43 |
| JLB017 | 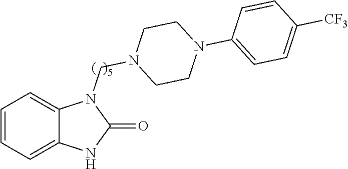 [SMILES: O=c1[nH]c2ccccc2n1CN1CCN(c2ccc(C(F)(F)F)cc2)CC1] | 7.19 | 5.05 |
| JLB018 | 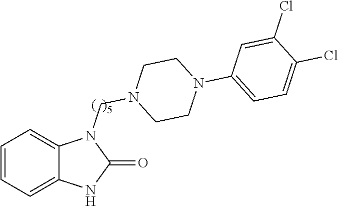 [SMILES: O=c1[nH]c2ccccc2n1CN1CCN(c2ccc(Cl)c(Cl)c2)CC1] | 1.22 | 0.86 |
| JLB019 | 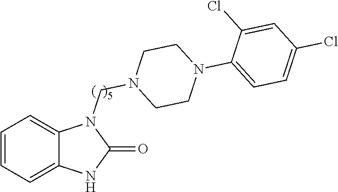 [SMILES: O=c1[nH]c2ccccc2n1CN1CCN(c2ccc(Cl)cc2Cl)CC1] | 17.5 | 12.3 |
| JLB020 | 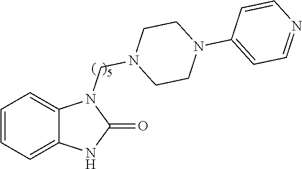 [SMILES: O=c1[nH]c2ccccc2n1CN1CCN(c2ccncc2)CC1] | 260 | 182 |
| JLB021 | 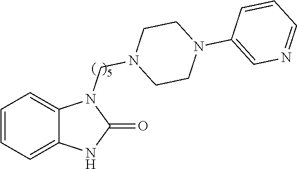 [SMILES: O=c1[nH]c2ccccc2n1CN1CCN(c2cccnc2)CC1] | 31.0 | 21.8 |
| JLB022 | 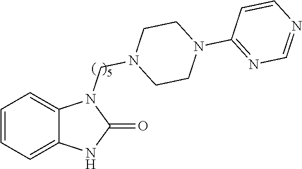 [SMILES: O=c1[nH]c2ccccc2n1CN1CCN(c2ccncn2)CC1] | 136 | 95.5 |
| JLB023 | 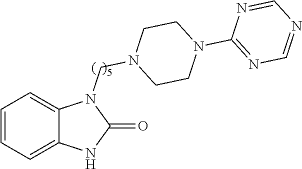 [SMILES: O=c1[nH]c2ccccc2n1CN1CCN(c2ncncn2)CC1] | 370 | 260 |
| JLB048 | 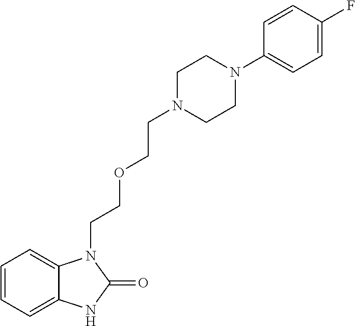 [SMILES: O=c1[nH]c2ccccc2n1CCOCCN1CCN(c2ccc(F)cc2)CC1] | 19.1 | 13.4 |
| JLB060 | 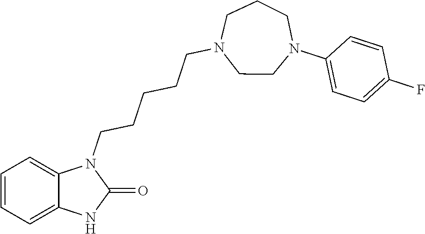 [SMILES: O=c1[nH]c2ccccc2n1CCCCCN1CCCN(c2ccc(F)cc2)CC1] | 0.98 | 0.69 |
| JLB061 | 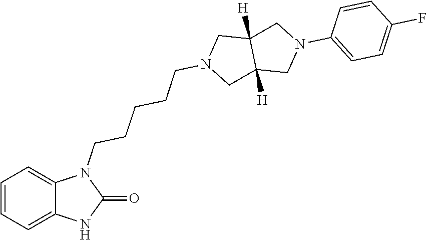 [SMILES: O=c1[nH]c2ccccc2n1CCCCCN1C[C@@H]2CN(c3ccc(F)cc3)C[C@@H]2C1] | 5.97 | 4.19 |
| JLB088 | 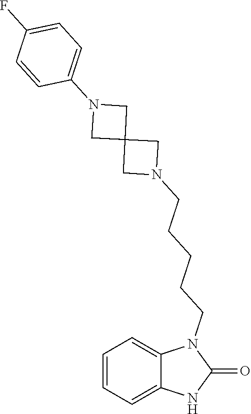 [SMILES: O=c1[nH]c2ccccc2n1CCCCCN1CC2(C1)CN(c1ccc(F)cc1)C2] | 174 | 122 |
| | Chemical Formula: C₂₃H₂₇FN₄O | | |
| | Molecular Weight: 394.4944 | | |
| JLB094 | 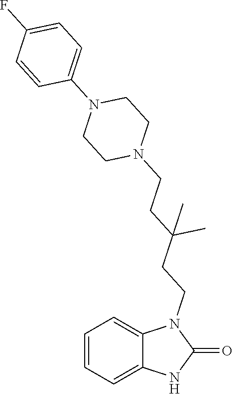 [SMILES: CC(C)(CCN1CCN(c2ccc(F)cc2)CC1)CCn1c(=O)[nH]c2ccccc21] | 2.7 | 1.90 |
| | Chemical Formula: C₂₄H₃₁FN₄O | | |
| | Molecular Weight: 410.5374 | | |
| JLB102 | 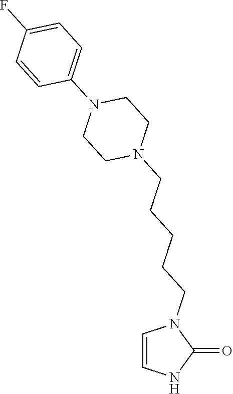 [SMILES: O=c1[nH]ccn1CCCCCN1CCN(c2ccc(F)cc2)CC1] | 17 | 11.9 |
| | Chemical Formula: C₁₈H₂₅FN₄O | | |
| | Molecular Weight: 332.4234 | | |
| JLB118 | 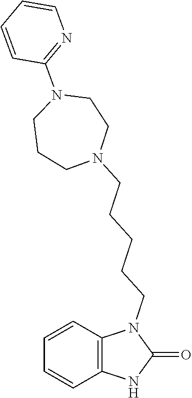 [SMILES: O=c1[nH]c2ccccc2n1CCCCCN1CCCN(c2ccccn2)CC1] | 69.7 | 48.9 |
| | Chemical Formula: C₂₂H₂₉N₅O | | |
| | Molecular Weight: 379.5080 | | |
| JLB119 | 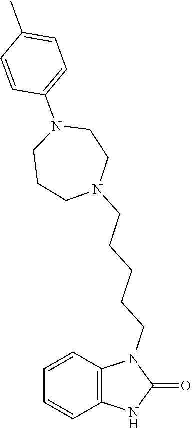 [SMILES: Cc1ccc(N2CCCN(CCCCCn3c(=O)[nH]c4ccccc43)CC2)cc1] | 24.2 | 17 |
| | Chemical Formula: C₂₄H₃₂N₄O | | |
| | Molecular Weight: 392.5470 | | |
| JLB137 | 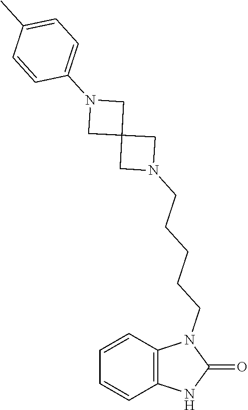 [SMILES: Cc1ccc(N2CC3(CN(CCCCCn4c(=O)[nH]c5ccccc54)C3)C2)cc1] | | |
| | Chemical Formula: C₂₄H₃₀N₄O | 687 | 482 |
| | Molecular Weight: 390.5310 | | |
| JLB138 | 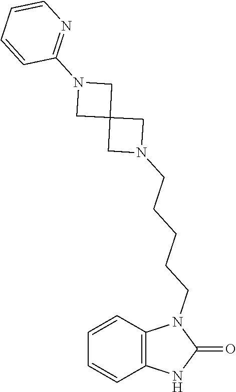 [SMILES: O=c1[nH]c2ccccc2n1CCCCCN1CC2(C1)CN(c1ccccn1)C2] | 5069 | 3560 |
| | Chemical Formula: C₂₂H₂₇N₅O | | |
| | Molecular Weight: 377.4920 | | |
| Clozapine | 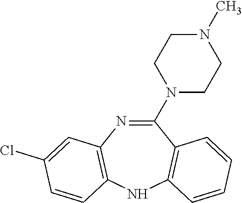 [SMILES: CN1CCN(C2=Nc3cc(Cl)ccc3Nc3ccccc32)CC1] | 3.86 | 2.71 |
| 5-CT | 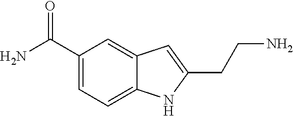 [SMILES: NCCc1cc2cc(C(N)=O)ccc2[nH]1] | 0.04 | 0.03 |
| LP211 | 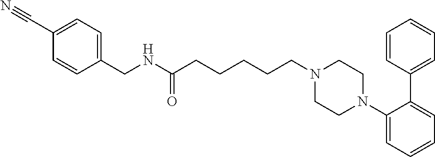 [SMILES: N#Cc1ccc(CNC(=O)CCCCCN2CCN(c3ccccc3-c3ccccc3)CC2)cc1] | 0.16 | 0.11 |
| E55888 | 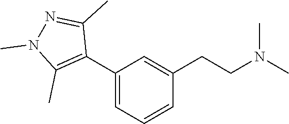 | 2.68 | 1.88 |

| | RLB 5-HT1A | | RLB 5-HT2A | | RLB 5-HT2Cedited | |
|---|---|---|---|---|---|---|
| Compound Id | IC50 (nM) | Ki (nM) | IC50 (nM) | Ki (nM) | IC50 (nM) | Ki (nM) |
| Serodolin | 203 | 102 | 16.9 | 2.8 | 1108 | 182 |
| MOA51 | 885 | 442 | 3.29 | 0.54 | 699 | 115 |
| JLB012 | 41.3 | 20.7 | 44.2 | 7.3 | 5249 | 863 |
| JLB016 | 482 | 241 | 47.5 | 7.8 | 1250 | 205 |
| JLB018 | 32.8 | 16.4 | 38.5 | 6.3 | 196 | 32 |
| JLB094 | 167 | 83.6 | 6.23 | 1.02 | 1103 | 181 |
| JLB060 | 850 | 425 | 137 | 23 | 902 | 148 |

| [symbol] | | [symbol] | | [symbol] | |
|---|---|---|---|---|---|
| IC50 (nM) | Ki (nM) | IC50 (nM) | Ki (nM) | IC50 (nM) | Ki (nM) |
| 1127 | 185 | 0.40 | 0.07 | 1390 | 226 |
| 1111 | 183 | 1.51 | 0.25 | 203 | 33.4 |
| >10000 | >5000 | 2.38 | 0.39 | 1013 | 166 |
| 5991 | 985 | 0.90 | 0.15 | 911 | 150 |
| 977 | 161 | 1.10 | 0.18 | 954 | 157 |
| 1825 | 300 | 2.56 | 0.42 | 964 | 158 |
| 8939 | 1469 | 1.49 | 0.24 | 1516 | 249 |

[symbol]

indicates data missing or illegible when filed

### 2. JLB060 Induced ERK Phosphorylation

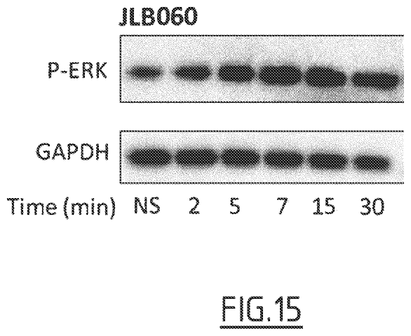

Considering previous studies that demonstrated the activation of ERK pathway downstream of Gs coupling to 5-HT₇R, the effect of JLB060 on ERK response was investigated. ERK phosphorylation was monitored by western blotting after treatment of cells with 10 μM of ligand at different times ranging from 2 to 30 minutes. However, unexpectedly, JLB060 was found to robustly induce ERK phosphorylation, dependent of the time of stimulation of the cells (FIG. 15).

### 3. MOA 51 Reduce Pain Behaviour in Writhing Test

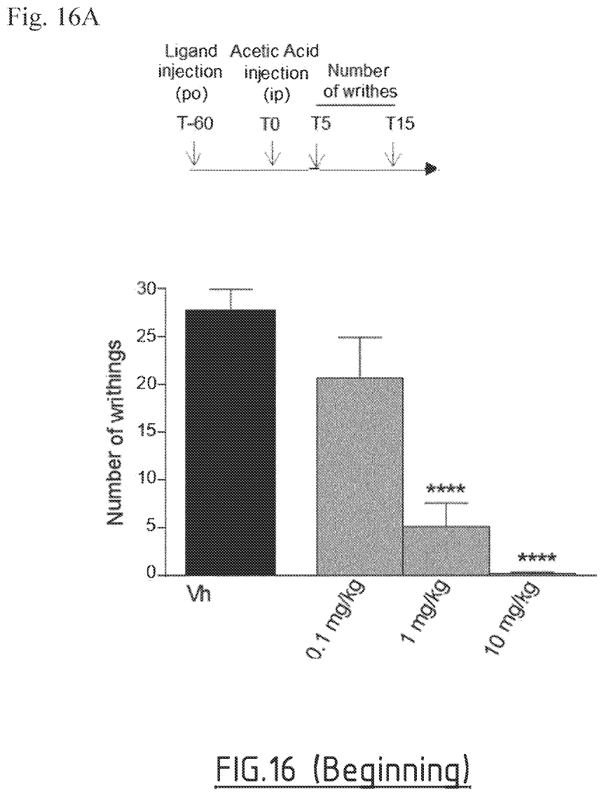

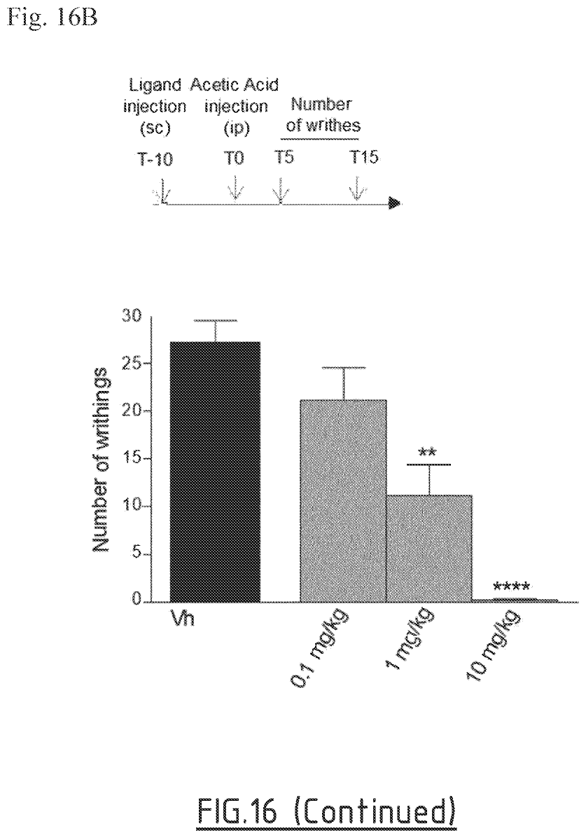

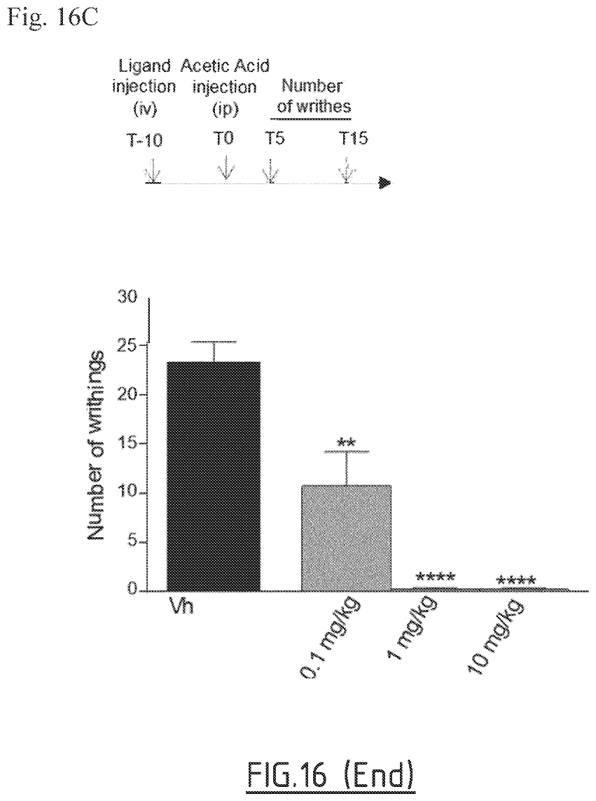

Analgesic activity was evaluated using the acetic acid abdominal constriction test (writhing test), a chemical model of visceral pain. We evaluated the dose-response effect of MOA51 following single oral, intravenous and subcutaneous administration of the compound one hour before injection of acetic acid. MOA51 administered by the oral route induced statistically significant dose-dependent decreases in the number of writhings at and above the dose of 1 mg/kg. By the intravenous route, MOA51 induced dose-dependent decreases in the number of writhings at and above the dose of 0.1 mg/kg. At the top dose of 10 mg/kg, no further acetic acid-induced writhings were observed. By the subcutaneous route, statistically significant decreases in the number of writhings at and above the dose of 1 mg/kg, with an absence of acetic acid-induced writhings at the top dose (FIG. 16).

### 4. Use of MOA51 in Thermoregulation

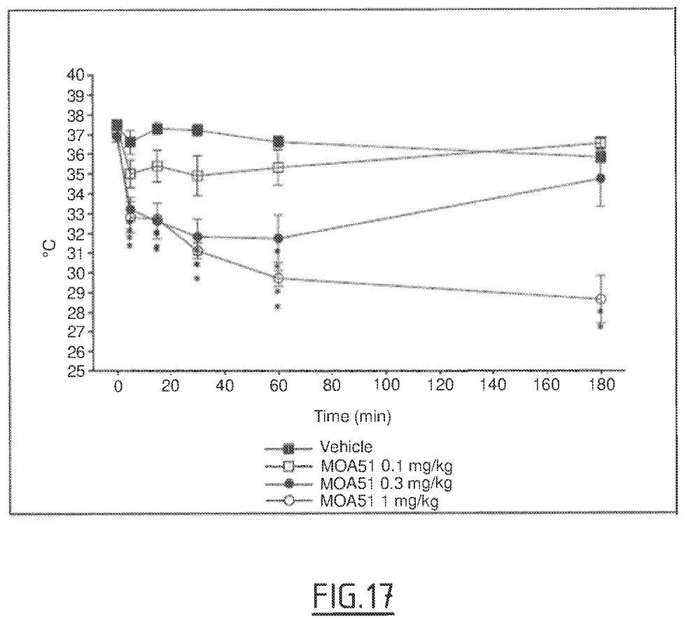

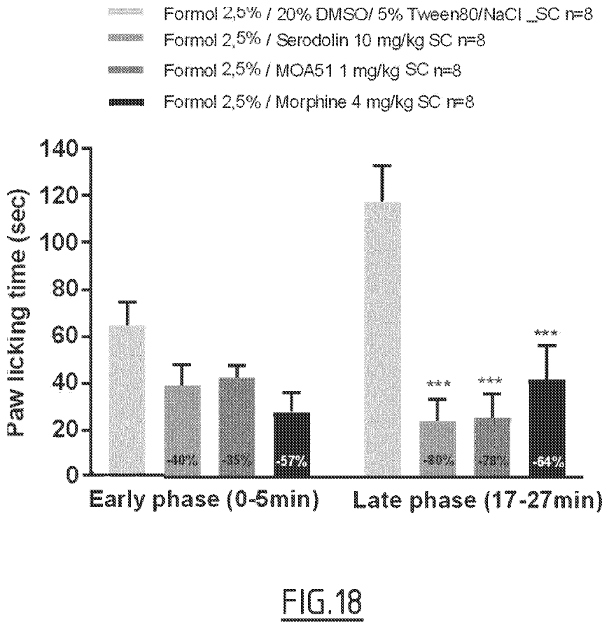

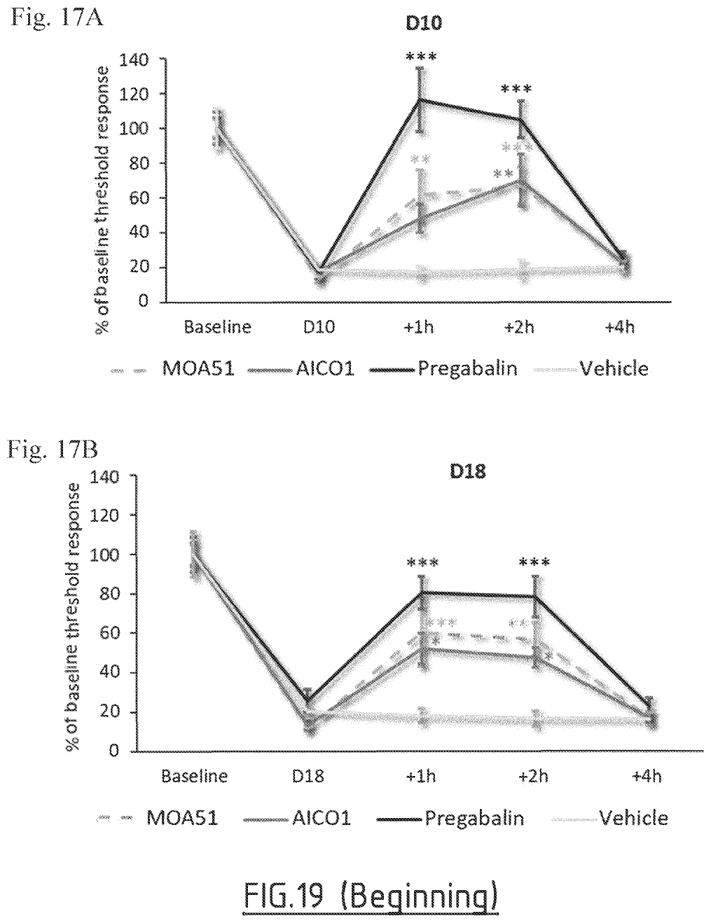

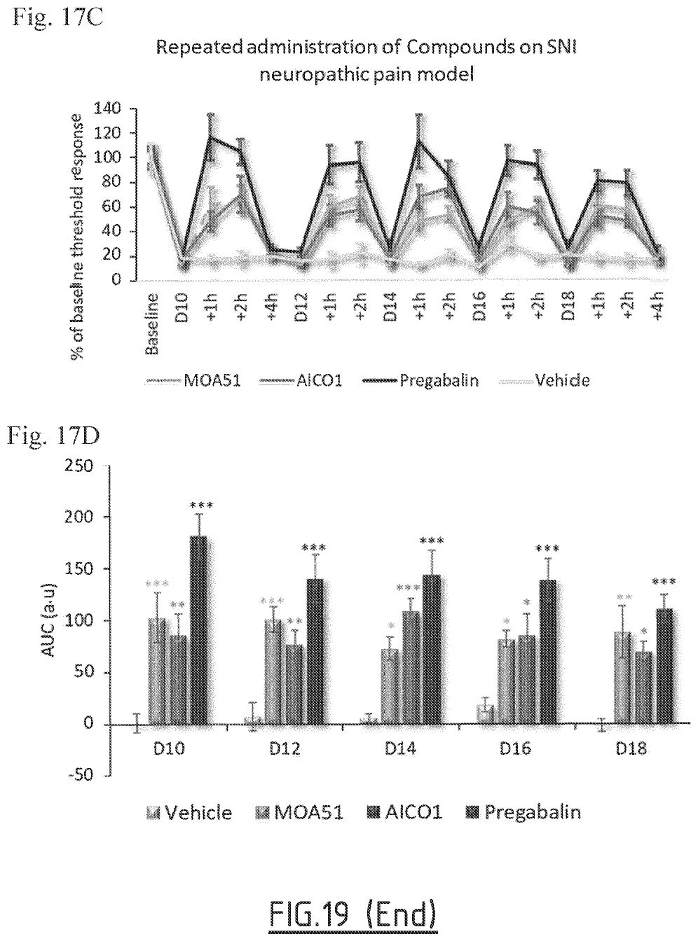

When MOA51 was administered at the dose of 1 mg/kg, a marked (Emax: −7.2° C. at 180 min) and long lasting decrease in body temperature was observed, statistically significant from 5 min post dosing up to the end of observations (180 min) (FIG. 19). At the intermediate dose of 0.3 mg/kg, MOA51 induced a clear-cut decrease in body temperature (Emax: −5.4° C. at 30 min), statistically significant up to 60 min post dosing. A tendency, not statistically significant, to a decrease in body temperature was observed (Emax: −2.3° C. at 30 min) from 5 min post dosing up to 60 min post dosing at the lowest dose of 0.1 mg/kg of MOA51. In conclusion, MOA51 induced a decrease in body temperature at and above the low dose of 0.3 mg/kg. This hypothermia was dose-dependent in intensity and duration. Therefore, MOA51 produces a significant and dose-dependent reduction in body temperature as previously reported with Serodolin (FIG. 17).

### 5. Effects of Serodolin and MOA51 in Formalin Test

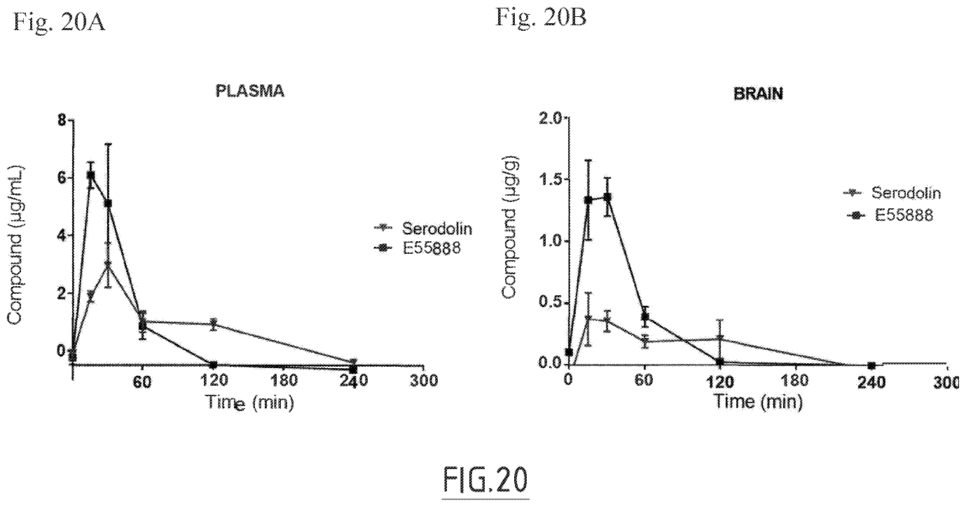

We evaluated the effect of Serodolin as well as MOA51 in rats in the formalin test, an acute and tonic pain model based on the use of a chemical stimulus. Subcutaneous injection of formalin into the right hindpaw produces a biphasic painful response of increasing and decreasing intensity for about 30 minutes after the injection. The initial phase of the response (early phase), likely caused by a burst of activity from C fibers, begins immediately after the formalin injection and lasts about 5 minutes. Although not significant, Serodolin, MOA51 and morphine have an inhibitory effect (−40%, −35% and −57% respectively) during the early phase. Interestingly Serodolin and MOA51 significantly inhibit licking (−80% and −78% respectively) and in a same extent as morphine (−64%) during the late phase of formalin-induced behaviours (FIG. 18). These results strongly suggest the potential analgesic effects of Serodolin and MOA51 for states of persistent pain in which tissue damage occurs.

### 6. Effects of Serodolin and MOA51 in Spinal Cord Injury Test.

Antalgic effect of MOA51 and AIC01 compounds. As shown in FIG. 19, 10 days after surgery, all mice presented a drastic decrease of mechanical response threshold. Pregabalin administration induced a significant increase of the mechanical response threshold compared to vehicle group (p<0.001 at 1 h and 2 h post-administration) after the first administration (FIG. 19A) or the last administration (FIG. 19B). MOA51 also presents an antalgic effect on neuropathic pain model. As shown in FIG. 19, MOA51 induced a significant increase in the mechanical threshold response at 1 h and 2 h, after the first administration (p=0.004 and p=0.001 respectively, FIG. 19A) or after the last administration (p<0.001 and p=0.001 respectively, FIG. 19B), in a similar manner. No abnormal behaviour was observed after administration of MOA51. However, we noticed that MOA51-injected mice often scratched at the neck immediately after injection for one or two minutes. AIC01 also shows an antalgic effect on neuropathic pain model. We observed a significant increase of the mechanical threshold response at 2 h after the first administration (p=0.101 at 1 h, and p<0.001 at 2 h) and at 1 h and 2 h after the last administration (p=0.010 and p=0.026, respectively).

Repetitive administration of MOA51 and AIC01 compounds. All compounds (Pregabalin, MOA51 and AIC01) and vehicle were administered for 9 consecutive days with a time-course measurement of the mechanical threshold responses every two days. Time-course curves look similar for every day, however, to better analyse and reveal possible variations in responses, Area Under the Curve (taking account of 1 h and 2 h measures) have been calculated and analysed for each day of measurements (FIG. 19C). AUC of Pregabalin presents significant differences compared to vehicle group for each analysed day. However, we observed a significant decrease of the analgesic effect of pregabalin between the first and the last administration (p<0.001). AUCs of MOA51 and AIC01 also show significant differences compared to vehicle group for each day (FIG. 19D). Even if both compounds have a less analgesic effect compared to Pregabalin, they didn't present any tolerance effect after repetitive administration.

### 7. Pharmacokinetic Profile of Serodolin Versus E55888, Used as Reference 5-HT7R Agonist in In Vivo Experiments.

The pharmacokinetics profile of both compounds were evaluated. The inventors used liquid chromatography tandem mass spectrometry (UPLC-MS/MS) method to perform PK study and measure Serodolin vs E55888 levels in vivo. The kinetics demonstrate the presence of both compounds for the same time period during experiments and their ability to pass the brain blood barrier. They show a maximum of detection at 15-30 minutes both in plasma and brain (2.9±0.8 μg/mL for Serodolin and 6.1±0.5 μg/mL for E55888 in plasma and 0.4±0.8 μg/mL for Serodolin and 1.4±0.2 μg/mL for E55888 in brain). However, whereas E55888 is eliminated after 120 min in both plasma and brain, Serodolin is still detected at this time and becomes undetectable after 240 min in plasma and brain (FIG. 20).

1. A compound having the following formula (I) 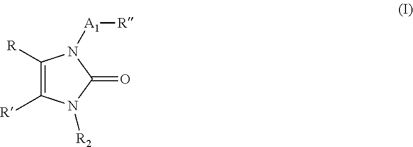
wherein:
R and R′ are, independently from each other, H or (C₁-C₆)alkyl groups, or form together with the carbon atoms carrying them a (C₆-C₁₀)aryl group;
said aryl group being optionally substituted with one or several substituents, said substituents being in particular selected from the group consisting of:
halogen;
(C₁-C₆)alkyl;
OH;
(C₁-C₆)alkoxy;
—NR_{d}Rₑ, R_{d} and Rₑ, independently from each other, being H or a (C₁-C₆)alkyl group;
aryl;
heteroaryl;
halo(C₁-C₆)alkyl group, such as CF₃;
—C(═O)—NR_{f}R_{g}, R_{f} and R_{g}, independently from each other, being H or a (C₁-C₆)alkyl group; and
—C(═O)—Rₕ, Rₕ being a (C₁-C₆)alkyl group;
R₂ is selected from the group consisting of:
H;
(C₁-C₆)alkyl group;
halo(C₁-C₆)alkyl group;
aryl; and
heteroaryl;
A₁ is a linker having the following formula (II): 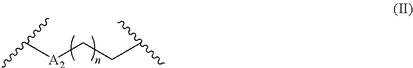
wherein:
n is an integer varying from 1 to 7; and
A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
R″ is:
either a group having the following formula (A-1): 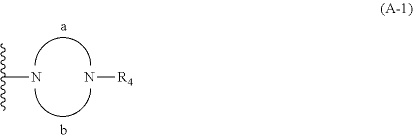
wherein:
the bonds “a” and “b” form a 4- to 10-membered saturated heterocycloalkyl group with the nitrogen atoms carrying them, said heterocycloalkyl group being optionally substituted for example with at least one substituent selected from (C₁-C₆)alkyl groups, and being selected from the monocyclic groups, bicyclic groups, fused bicycles and spiro-type rings; and
R₄ is selected from the optionally substituted (C₆-C₁₀)aryl and heteroaryl groups;
either a group having the following formula (A-2): 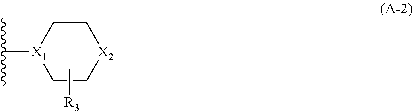
wherein:
X₁ is —N— or —CH—;
X₂ is selected from the group consisting of:
a group —X₁—R₄, X₁ being as defined above and R₄ being selected from the optionally substituted (C₆-C₁₀)aryl and heteroaryl groups; and
a group —CH—CO—Ar, Ar having the below formula (III): 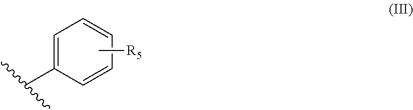
R₅ being selected from the group consisting of:
H;
halogen;
(C₁-C₆)alkyl;
halo(C₁-C₆)alkyl;
hetero(C₁-C₆)alkyl;
OH;
(C₁-C₆)alkoxy;
halo(C₁-C₆)alkoxy;
CN;
—C(═O)—Rᵢ, Rᵢ being a (C₁-C₆)alkyl group;
—SO₂—NRⱼRₖ, Rⱼ and Rₖ, independently from each other, being H or a (C₁-C₆)alkyl group;
—NR_{b}R_{c}, R_{b} and Rₑ, independently from each other, being H or a (C₁-C₆)alkyl group; and
optionally substituted (C₆-C₁₀)aryl and heteroaryl, said aryl or heteroaryl being possible fused with the phenyl ring carrying them; and
R₃ is selected from the group consisting of:
H;
(C₁-C₆)alkyl group; and
hetero(C₁-C₆)alkyl group;
or its pharmaceutically acceptable salts, racemates, diastereomers or enantiomers,
for use in the treatment of a brain disorder involving modified 5-HT7R-mediated signaling or for use to induce hypothermia.

2. The compound for the use of claim 1, in the treatment of pain or inflammation or in the treatment of multiple sclerosis, or for use to induce hypothermia.

3. A compound having the following formula (I′): 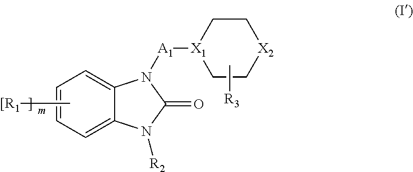
wherein:
m is an integer comprised from 1 to 4;
each R₁, identical or different, is selected from the group consisting of:
H;
halogen;
(C₁-C₆)alkyl;
OH;
(C₁-C₆)alkoxy;
—NR_{d}Rₑ, R_{d} and Rₑ, independently from each other, being H or a (C₁-C₆)alkyl group;
aryl;
heteroaryl;
halo(C₁-C₆)alkyl group such as CF₃;
—C(═O)—NR_{f}R_{g}, R_{f} and R_{g}, independently from each other, being H or a (C₁-C₆)alkyl group; and
—C(═O)—Rₕ, Rₕ being a (C₁-C₆)alkyl group;
—R₂ is selected from the group consisting of:
H;
(C₁-C₆)alkyl group;
halo(C₁-C₆)alkyl group;
aryl; and
heteroaryl;
A₁, X₁, X₂, and R₃ are as defined in claim 1;
or its pharmaceutically acceptable salts, racemates, diastereomers or enantiomers,
for use in the treatment of pain or in the treatment of multiple sclerosis, or for use to induce hypothermia.

4. The compound for the use of claim 1, wherein said compound has the following formula (IV): 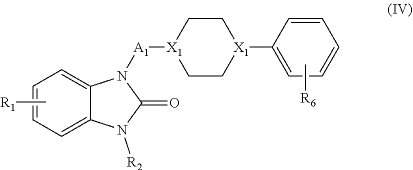
wherein:
R₁, R₂, A₁, and X₁ are as defined in claim 1; and
R₆ is selected from the group consisting of: H, (C₁-C₆)alkyl, —OH, (C₁-C₆)alkoxy, halogen, thio(C₁-C₆)alkyl, halo(C₁-C₆)alkyl, halo(C₁-C₆)alkoxy, and
—NR_{b}R_{c}, R_{b} and Rₑ, independently from each other, being H or a (C₁-C₆)alkyl group.

5. The compound for the use of claim 4, wherein said compound has the formula (IV) wherein X₁ is —N—.

6. The compound for the use of claim 1, wherein said compound has the following formula (V): 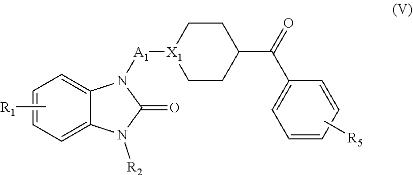
wherein R₁, R₂, A₁, X₁, and R₅ are as defined in claim 1.

7. The compound for the use of claim 6, wherein said compound has the formula (V) wherein X₁ is —N—.

8. The compound for the use of claim 2, wherein R₁ is H or a halogen atom.

9. The compound for the use of claim 1, wherein R₂ is H or a (C₁-C₆)alkyl group.

10. The compound for the use of claim 1, wherein A₁ is a (C₂-C₇)alkylene radical.

11. The compound for the use of claim 1, wherein said compound has the following formula (VI): 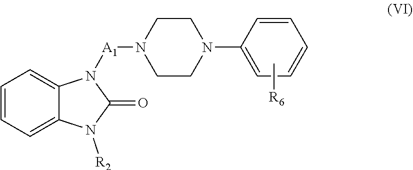
wherein:
R₂ and A₁ are as defined in claim 1; and
R₆ is selected from the group consisting of: H, —OH, (C₁-C₆)alkoxy, halogen, and thio(C₁-C₆)alkyl.

12. The compound for the use of claim 11, wherein said compound has the formula (VI) wherein:
R₂ is H or a (C₁-C₆)alkyl group, such as a n-butyl group; and/or
A₁ is a C₄ or C₅ alkylene radical.

13. The compound for the use of claim 1, wherein said compound has the following formula (VII): 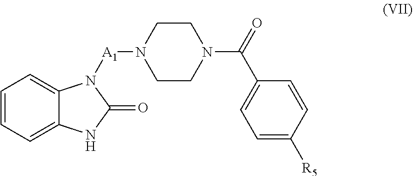
wherein A₁ and R₅ are as defined in claim 1.

14. The compound for the use of claim 13, wherein said compound has the formula (VII) wherein:
R₅ is halogen, and preferably F; and/or
A₁ is a (C₂-C₇)alkylene radical.

15. The compound for the use of claim 1, wherein the pain is selected from the group consisting of: pain from thermic, mechanic, or inflammatory stimulus, acute and tonic pain, inflammatory pain, visceral pain, neuropathic pain, and post-operative pain.

16. A compound having the following formula (I-1) 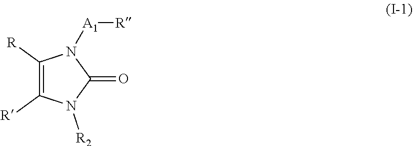
wherein:
R and R′ are, independently from each other, H or (C₁-C₆)alkyl groups, or form together with the carbon atoms carrying them a (C₆-C₁₀)aryl group;
said aryl group being optionally substituted with one or several substituents, said substituents being in particular selected from the group consisting of:
halogen;
(C₁-C₆)alkyl;
OH;
(C₁-C₆)alkoxy;
—NR_{d}Rₑ, R_{d} and Rₑ, independently from each other, being H or a (C₁-C₆)alkyl group;
aryl;
heteroaryl;
halo(C₁-C₆)alkyl group, such as CF₃;
—C(═O)—NR_{f}R_{g}, R_{f} and R_{g}, independently from each other, being H or a (C₁-C₆)alkyl group; and
—C(═O)—Rₕ, Rₕ being a (C₁-C₆)alkyl group;
R₂ is selected from the group consisting of:
H;
(C₁-C₆)alkyl group;
halo(C₁-C₆)alkyl group;
aryl; and
heteroaryl;
A₁ is a linker having the following formula (II): 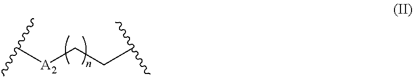
wherein:
n is an integer varying from 1 to 7; and
A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
R″ is a group having the following formula (A-1): 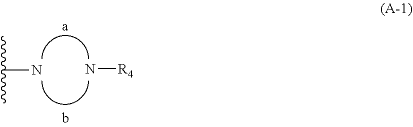
wherein:
the bonds “a” and “b” form a 4- to 10-membered saturated heterocycloalkyl group with the nitrogen atoms carrying them, said heterocycloalkyl group being optionally substituted for example with at least one substituent selected from (C₁-C₆)alkyl groups, and being selected from the monocyclic groups, bicyclic groups, fused bicycles and spiro-type rings; and
R₄ is selected from the optionally substituted heteroaryl groups;
or its pharmaceutically acceptable salts, racemates, diastereomers or enantiomers.

17. The compound of claim 16 having the following formula (I-1), wherein R and R′ form together with the carbon atoms carrying them a (C₆-C₁₀)aryl group, in particular a fused phenyl group.

18. The compound of claim 16, having the following formula (I-1), wherein R₂ is H.

19. The compound of claim 16, having the following formula (I-2): 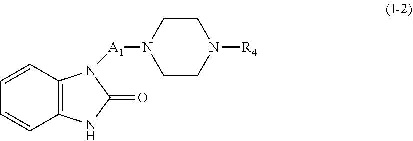
A₁ and R₄ being as defined in claim 16.

20. A compound having the formula (I-3): 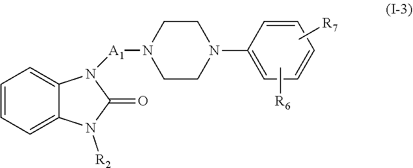
wherein:
R₂ is selected from the group consisting of:
H;
(C₁-C₆)alkyl group;
halo(C₁-C₆)alkyl group;
aryl; and
heteroaryl;
A₁ is a linker having the following formula (II): 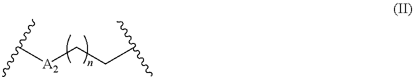
wherein:
n is an integer varying from 1 to 7; and
A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
R₆ is selected from the group consisting of: —OH, (C₁-C₆)alkoxy, (C₁-C₆)alkyl, halogen, and thio(C₁-C₆)alkyl, and
R₇ is halogen;
or its pharmaceutically acceptable salts, racemates, diastereomers or enantiomers.

21. A compound having the following formula (I-4): 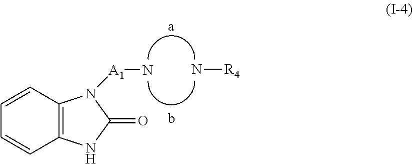
wherein:
A₁ is a linker having the following formula (II): 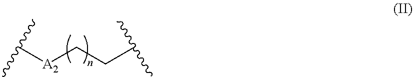
wherein:
n is an integer varying from 1 to 7; and
A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
the bonds “a” and “b” form a 4- to 10-membered saturated heterocycloalkyl group with the nitrogen atoms carrying them, said heterocycloalkyl group being optionally substituted for example with at least one substituent selected from (C₁-C₆)alkyl groups, and being selected from the monocyclic groups, bicyclic groups, fused bicycles and spiro-type rings; and
R₄ is selected from the optionally substituted (C₆-C₁₀)aryl and heteroaryl groups;
or its pharmaceutically acceptable salts, racemates, diastereomers or enantiomers.

22. A compound having the following formula (I-5): 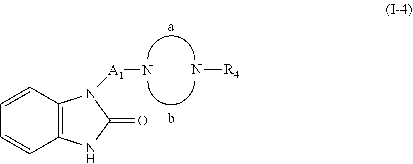
wherein:
A₁ is a linker comprising from 3 to 10 carbon atoms, wherein possibly at least one carbon atom of A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
the bonds “a” and “b” form a 4- to 10-membered saturated heterocycloalkyl group with the nitrogen atoms carrying them, said heterocycloalkyl group being optionally substituted for example with at least one substituent selected from (C₁-C₆)alkyl groups, and being selected from the monocyclic groups, bicyclic groups, fused bicycles and spiro-type rings; and
R₄ is selected from the optionally substituted (C₆-C₁₀)aryl and heteroaryl groups;
or its pharmaceutically acceptable salts, racemates, diastereomers or enantiomers.

23. A compound having the following formula (I-6): 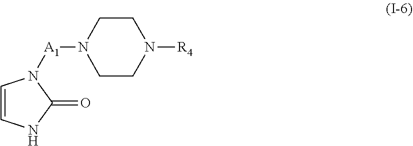
wherein:
A₁ is a linker having the following formula (II): 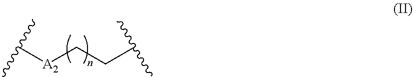
wherein:
n is an integer varying from 1 to 7; and
A₂ is a bond or a C₂ divalent radical, possibly substituted with at least one substituent selected from the group consisting of: (C₁-C₆)alkyl, (C₃-C₇)cycloalkyl, and hetero(C₁-C₆)alkyl, wherein possibly at least one carbon atom of A₂ or A₁ is replaced with a heteroatom such as —O—, —S— or —NRₐ—, Rₐ being H or a (C₁-C₆)alkyl group;
and wherein A₁ is possibly substituted with at least one substituent selected from the group consisting of: (C₁-C₆)alkyl, (C₃-C₇)cycloalkyl, and hetero(C₁-C₆)alkyl;
R₄ is selected from the optionally substituted (C₆-C₁₀)aryl and heteroaryl groups;
or its pharmaceutically acceptable salts, racemates, diastereomers or enantiomers.
